(19) United States
(12) Patent Application Publication
(10) Pub. No.: US 2025/0032284 A1
(43) Pub. Date: Jan. 30, 2025

(54) DELIVERY SYSTEMS FOR AORTIC ARCH REPAIR DEVICES, AND ASSOCIATED DEVICES AND METHODS

(71) Applicant: Mavericks Endo, Inc., Menlo Park, CA (US)

(72) Inventors: Richard Roy Newhauser, Belmont, CA (US); G Ray Martin, Redwood City, CA (US); Steven Kim, Los Altos, CA (US); Tony Le, Sunnyvale, CA (US); Morgan Jawitz, Mountain View, CA (US); Simon Nguyen, San Jose, CA (US); Hanson Gifford, Woodside, CA (US); Brian Tachibana, Danville, CA (US)

(21) Appl. No.: 18/784,740

(22) Filed: Jul. 25, 2024

Related U.S. Application Data
(60) Provisional application No. 63/515,517, filed on Jul. 25, 2023.

(51) Int. Cl. A61F 2/95; A61M 25/00

(52) U.S. Cl. CPC A61F 2/9517; A61M 25/0026; A61M 25/0074

(57) ABSTRACT

Delivery systems for delivering an aortic repair device to treat a diseased portion of the aorta of a patient at or near the aortic arch and associated devices and methods are disclosed herein. A delivery system configured in accordance with embodiments of the present technology can include an outer catheter, an inner catheter assembly extending at least partially through the outer catheter, a handle, a tip capture mechanism configured to releasably secure an end portion of the aortic repair device, and a release wire actuatable to release the end portion from the tip capture mechanism. The handle can be actuated to retract the outer catheter relative to the inner catheter assembly to deploy the aortic repair device. The handle can also be actuated to advance a portion of the inner catheter assembly relative to the outer catheter to reorient and position the end portion of the aortic repair device.

### CROSS-REFERENCE TO RELATED APPLICATIONS

This application claims the benefit of U.S. Provisional Patent application No. 63/515,517, filed Jul. 25, 2023, and titled “Delivery Systems for Aortic Arch Repair Devices, and Associated Devices and Methods”, the disclosure of which is incorporated herein by reference in its entirety.

### TECHNICAL FIELD

The present technology generally relates to delivery systems for implanting aortic repair devices at least partially within a diseased aorta for repairing the diseased aorta, such as delivery systems for implanting aortic repair devices within the thoracic (e.g., proximal) aorta to treat aneurysms and dissections in the ascending aorta and/or the aortic arch.

### BACKGROUND

Aneurysms, dissections, penetrating ulcers, intramural hematomas, and/or transections may occur in blood vessels, and most typically occur in the aorta and peripheral arteries. A diseased region of the aorta may extend into areas having vessel bifurcations or segments of the aorta from which smaller “branch” arteries extend.

The diseased region of the aorta and other vessels can be bypassed with a stent graft placed inside the vessel to span the diseased region. The stent graft can effectively seal off the diseased region from further exposure to blood flow, inhibiting or preventing the aneurysm, dissection, or other type of diseased region from worsening. However, the use of stent grafts to internally bypass a diseased region of a vessel is not without challenges. In particular, care must be taken so that the stent graft does not cover or occlude critical branch vessels, yet the stent graft must adequately seal against the healthy regions of the vessel wall and remain open to provide a flow conduit for blood to flow past the diseased region.

### BRIEF DESCRIPTION OF THE DRAWINGS

Many aspects of the present disclosure can be better understood with reference to the following drawings. The components in the drawings are not necessarily to scale. Instead, emphasis is placed on clearly illustrating the principles of the present disclosure.

FIGS. 1A and 1B are side views of a diseased aorta and surrounding anatomy in which an aortic repair device can be implanted by a delivery system in accordance with embodiments of the present technology.

FIGS. 2A-2D are side cross-sectional views of a diseased aorta in which an aortic repair device can be implanted by a delivery system in accordance with embodiments of the present technology.

FIG. 3A is a side view of a delivery system configured in accordance with embodiments of the present technology.

FIG. 3B is a side view of segments of an inner catheter assembly of the delivery system of FIG. 3A in accordance with embodiments of the present technology.

FIG. 3C is a perspective side view of an aortic repair device secured to a distal portion of the delivery system of FIG. 3A in accordance with embodiments of the present technology.

FIG. 3D is a perspective side view of a connector assembly that can be incorporated into the delivery system of FIG. 3A in accordance with embodiments of the present technology.

FIGS. 3E-3G are side views of the delivery system of FIG. 3A in a delivery position, a deployed position, and a retracted position, respectively, in accordance with embodiments of the present technology.

FIG. 4A is a side view of the aortic repair device of FIG. 3C implanted within an aorta after implantation via the delivery system of FIG. 3A in accordance with embodiments of the present technology.

FIG. 4B is a side view of an aortic repair device implanted within an aorta in accordance with embodiments of the present technology.

FIG. 4C is a side view of an aortic repair device implanted within an aorta in accordance with embodiments of the present technology.

FIG. 5A is a perspective top view of a delivery system in accordance with additional embodiments of the present technology.

FIGS. 5B and 5C are enlarged perspective views of a handle of the delivery system of FIG. 5A in accordance with embodiments of the present technology.

FIGS. 6A and 6B are a perspective top view and a perspective side view, respectively, of a delivery system in accordance with additional embodiments of the present technology.

FIG. 6C is an enlarged perspective view of a handle of the delivery system of FIGS. 6A and 6B in accordance with embodiments of the present technology.

FIGS. 6D and 6E are perspective views of a distal portion and a proximal portion, respectively, of the handle of the delivery system of FIGS. 6A and 6B in accordance with embodiments of the present technology.

FIG. 7A is a perspective view of a distal portion of the delivery system of FIGS. 3A-3G partially secured to an aortic repair device in accordance with embodiments of the present technology.

FIG. 7B is a schematic end-on view of the distal portion of the delivery system and the aortic repair device of FIG. 7A in accordance with embodiments of the present technology.

FIGS. 7C and 7D are perspective side views of the distal portion of the delivery system of FIG. 7A after compressing the aortic repair device within an outer catheter in accordance with embodiments of the present technology.

FIG. 7E is a schematic end-on view of the distal portion of the delivery system and the aortic repair device of FIGS. 7C and 7D in accordance with embodiments of the present technology.

FIGS. 8A-8C are perspective side views of the delivery system during different stages of deployment of the aortic repair device of FIGS. 7A-7E within a lumen of a vessel in accordance with embodiments of the present technology.

FIG. 9 is a schematic end-on view of a leading end portion of an aortic repair device secured to a loop of a leading tip capture mechanism of a delivery system in accordance with embodiments of the present technology.

FIG. 10A is a schematic end-on view of a leading end portion of an aortic repair device secured to a loop of a leading tip capture mechanism of a delivery system in accordance with embodiments of the present technology.

FIG. 10B is a side view of the delivery system of FIGS. 3A-3G positioned at least partially within an aorta and configured to deploy the aortic repair device of FIG. 10A within the aorta in accordance with embodiments of the present technology.

FIG. 11 is a schematic end-on view of a leading end portion of an aortic repair device secured to multiple loops of a leading tip capture mechanism of a delivery system in accordance with embodiments of the present technology.

FIG. 12A is a schematic end-on view of a leading end portion of an aortic repair device secured to multiple loops of a leading tip capture mechanism of a delivery system in accordance with embodiments of the present technology.

FIGS. 12B-12D are perspective side views of the delivery system of FIGS. 3A-3G during different stages of releasing the leading end portion of the aortic repair device of FIG. 12A within an aorta in accordance with embodiments of the present technology.

FIG. 13A is a perspective view of a distal portion of the delivery system of FIGS. 3A-3G secured to an aortic repair device in accordance with embodiments of the present technology.

FIG. 13B is a perspective view of a distal portion of the delivery system of FIGS. 3A-3G secured to the aortic repair device of FIG. 13A in accordance with additional embodiments of the present technology.

FIG. 14 is a schematic end-on view of a leading end portion of an aortic repair device secured to multiple loops of a leading tip capture mechanism of a delivery system in accordance with embodiments of the present technology.

FIG. 15 is an isometric view of a distal portion of a delivery system including a leading tip capture mechanism in accordance with embodiments of the present technology.

FIG. 16 is a perspective view of a distal portion of a delivery system including a leading tip capture mechanism secured to an aortic repair device in accordance with embodiments of the present technology.

FIGS. 17A-17D are perspective views of a distal portion of the delivery system and the aortic repair device of FIG. 16 during different stages of releasing a leading stent of the aortic repair device in accordance with embodiments of the present technology.

FIG. 18 is an end-on view of a leading end portion of an aortic repair device secured to a leading tip capture mechanism of a delivery system in accordance with embodiments of the present technology.

FIG. 19 is a side view of an aortic repair device secured to a leading tip capture mechanism of a delivery system in accordance with embodiments of the present technology.

FIGS. 20A and 20B are side views of a leading tip capture mechanism of a delivery system in accordance with embodiments of the present technology.

FIG. 21 is a side view of an aortic repair device secured to a leading tip capture mechanism of a delivery system in accordance with embodiments of the present technology.

FIGS. 22A and 22B are schematic end-on views of a leading end portion of an aortic repair device secured to a tip capture mechanism of a delivery system in accordance with embodiments of the present technology.

FIG. 23 is a perspective side view of a distal portion of the delivery system of FIGS. 3A-3G secured to an aortic repair device in accordance with embodiments of the present technology.

FIG. 24 is a perspective view of a distal portion of the delivery system of FIGS. 3A-3G secured to an aortic repair device in accordance with embodiments of the present technology.

FIG. 25A is a perspective view of a distal portion of the delivery system of FIGS. 3A-3G secured to an aortic repair device in accordance with embodiments of the present technology.

FIG. 25B is a side view of an inner catheter assembly of the delivery system of FIGS. 3A-3G in accordance with embodiments of the present technology.

FIGS. 26A and 26B are side views of the delivery system of FIGS. 3A-3G positioned at least partially within an aorta and configured to deploy an aortic repair device in accordance with embodiments of the present technology.

FIG. 27A is a perspective side view of a distal portion of an inner catheter assembly of the delivery system of FIGS. 3A-3G in accordance with embodiments of the present technology.

FIGS. 27B and 27C are side views of a first stage and a second stage, respectively, of a procedure to implant an aortic repair device within an aorta of a patient using the delivery system of FIG. 27A in accordance with embodiments of the present technology.

FIGS. 28A-28C are perspective side views of a distal portion of an inner catheter assembly of the delivery system of FIGS. 3A-3G in a first position, a second position, and a third position, respectively, in accordance with embodiments of the present technology.

FIG. 28D is a perspective side view of a sleeve of an inner catheter of the inner catheter assembly of FIGS. 28A-28C in accordance with embodiments of the present technology.

FIG. 28E is a perspective side view of a sleeve of the inner catheter of the inner catheter assembly of FIGS. 28A-28C in accordance with additional embodiments of the present technology.

FIG. 28F is a side view of a distal portion of the inner catheter assembly 310 of FIGS. 28A-28C including a leading tip capture mechanism 2842 in accordance with embodiments of the present technology.

FIG. 28G is a perspective side view of the distal portion of the delivery system of FIGS. 28A-28C with the inner catheter assembly in the third position and positioned within an outer catheter in accordance with embodiments of the present technology.

FIG. 28H is a side view of the delivery system of FIGS. 28A-28G positioned at least partially within an aorta and configured to deploy an aortic repair device in accordance with embodiments of the present technology.

FIG. 29A is a perspective side view of a distal portion of the delivery system of FIGS. 3A-3G in accordance with embodiments of the present technology.

FIG. 29B is a side view of the delivery system of FIG. 29A positioned at least partially within an aorta and configured to deploy an aortic repair device in accordance with embodiments of the present technology.

FIGS. 30A and 30B are perspective side views of a distal portion of an inner catheter assembly of the delivery system of FIGS. 3A-3G in a first position and a second position, respectively, in accordance with embodiments of the present technology.

FIGS. 31A-31C are side views of the inner catheter assembly of FIGS. 30A and 30B inserted within an aorta of a patient over a guidewire and in a first position, a second position, and a third position, respectively, in accordance with embodiments of the present technology.

FIG. 32A is a schematic side view of a distal portion of the delivery system secured to the aortic repair device of FIGS. 3A-3G in accordance with embodiments of the present technology.

FIG. 32B is an enlarged side view of a portion of the delivery system and the aortic repair device of FIG. 32A in accordance with embodiments of the present technology.

FIGS. 32C and 32D are side views of a first stage and a second stage, respectively, of a procedure to implant the aortic repair device within an aorta of a patient using the delivery system of FIGS. 32A and 32B in accordance with embodiments of the present technology.

FIGS. 33A-33D are side views of different stages of a procedure to implant an aortic repair device within an aorta of a patient using the delivery system of FIGS. 3A-3G in accordance with embodiments of the present technology.

FIG. 34A is an enlarged side view of a portion of the delivery system and the aortic repair device of FIGS. 34A and 34B in accordance with embodiments of the present technology.

FIGS. 34B-34E are side views of different stages of a procedure to implant the aortic repair device within an aorta of a patient using the delivery system of FIGS. 3A-3G in accordance with embodiments of the present technology.

FIG. 35 is an isometric view of a funnel device in accordance with embodiments of the present technology.

FIGS. 36A and 36B are a front view and an isometric view, respectively, of a funnel device in accordance with additional embodiments of the present technology.

FIGS. 37A-37C are a side view, a perspective side view, and another perspective side view, respectively, of a distal portion of the delivery system of FIGS. 3A-3G secured to an aortic repair device in accordance with embodiments of the present technology.

FIGS. 38A, 38C, and 38E are side views illustrating different stages of securing a first side portion of the aortic repair device of FIG. 3C to the delivery system of FIGS. 3A-3G in accordance with embodiments of the present technology.

FIGS. 38B, 38D, and 38F are side views illustrating corresponding stages of securing a second side portion of the aortic repair device of FIG. 3C to the delivery system of FIGS. 3A-3G in accordance with embodiments of the present technology.

FIGS. 39A-39C illustrate different stages of deploying the aortic repair device of FIGS. 38A-38D having constraining fibers with the delivery system of FIGS. 3A-3G in accordance with embodiments of the present technology.

FIG. 40 is a side view of the aortic repair device of FIGS. 38A-39C secured to the delivery system of FIGS. 3A-3G in accordance with additional embodiments of the present technology.

FIG. 41A is an isometric view of a distal portion of a delivery system including a leading tip capture mechanism in accordance with embodiments of the present technology.

FIG. 41B is a perspective view of a distal portion of the leading tip capture mechanism of the delivery system of FIG. 41A releasably secured to an aortic repair device in accordance with embodiments of the present technology.

FIG. 42A is a perspective side view of a release wire assembly that can be used in a delivery system in accordance with embodiments of the present technology.

FIG. 42B is an enlarged side view of a proximal portion of the release wire assembly of FIG. 42A in accordance with embodiments of the present technology.

FIG. 43A is an isometric view of a leading stent of an aortic repair device secured to a leading tip capture mechanism of a delivery system in accordance with embodiments of the present technology.

FIGS. 43B and 43C are isometric views of the leading tip capture mechanism of FIG. 43A in a first release position and a second release position, respectively, in accordance with embodiments of the present technology.

FIG. 43D is an isometric view of the leading tip capture mechanism of FIG. 43A in accordance with additional embodiments of the present technology.

FIGS. 44A-44I are side views of different stages of a procedure to implant an aortic repair device within an aorta of a patient using the delivery system of FIGS. 28A-28G in accordance with embodiments of the present technology.

FIG. 45A is a side view of a delivery system including a leading tip capture mechanism and configured in accordance with embodiments of the present technology.

FIGS. 45B and 45C are enlarged views of a portion of the delivery system of FIG. 45A illustrating additional features of the leading tip capture mechanism in accordance with embodiments of the present technology.

FIG. 46A is a side view of a delivery system including a forward drive mechanism and configured in accordance with embodiments of the present technology.

FIG. 46B is an enlarged view of a proximal end portion of the delivery system of FIG. 46A in accordance with embodiments of the present technology.

FIG. 46C is a cross-sectional view of a portion of the delivery system of FIG. 46A in accordance with embodiments of the present technology.

FIGS. 46D and 46E are enlarged views of portions of the cross-sectional view of FIG. 46C, illustrating additional details of the delivery system of FIG. 46A in accordance with embodiments of the present technology.

FIG. 47 is a cross-sectional view of a portion of another forward drive mechanism that can be used with the delivery system of FIG. 46A and configured in accordance with embodiments of the present technology.

FIGS. 48A-48F illustrate different stages of a procedure to implant an aortic repair device within an aorta of a patient using the delivery system of FIGS. 46A-46E in accordance with embodiments of the present technology.

FIGS. 49A-49C illustrate different stages of a procedure to implant an aortic repair device within an aorta and a brachiocephalic artery of a patient using the delivery system of FIGS. 46A-46E in accordance with embodiments of the present technology.

FIGS. 50A and 50B illustrate different states of a procedure to implant a modular aortic repair device within an aorta of a patient using the delivery system of FIGS. 46A-46E in accordance with embodiments of the present technology.

### DETAILED DESCRIPTION

The present technology is directed to delivery systems for delivering an aortic repair device to treat a diseased aorta (e.g., thoracic aorta) of a patient, such as a human patient, and associated devices and methods. In some embodiments, for example, a delivery system includes an outer catheter, an inner catheter assembly extending at least partially through the outer catheter, and a handle operably coupled to the outer catheter and/or the inner catheter assembly. An aortic repair device can be releasably secured to the inner catheter assembly and positioned within the outer catheter in a delivery position. The outer catheter and the inner catheter assembly can be advanced to a target location within an aorta in the delivery position with the aortic repair device compressed within the outer catheter. The target location can be a location within the thoracic aorta, such as in or near the ascending aorta, the aortic arch, and/or the descending thoracic aorta, proximate to a diseased portion of the aorta, such as a dissection, tear, and/or aneurysm. The handle can be actuated to retract the outer catheter relative to the inner catheter to deploy the aortic repair device at the target location.

In some embodiments, the inner catheter assembly includes a leading tip capture mechanism configured to be releasably secured to a leading end portion of the aortic repair device. The leading tip capture mechanism can capture and retain all or a portion of the leading end portion after the aortic repair device is deployed from the outer catheter. The leading tip capture mechanism can be actuated by for example, retracting one or more release wires of the delivery system to selectively release the leading end portion of the aortic repair device. In some aspects of the present technology, during deployment of the aortic repair device, the leading tip capture mechanism can tension the aortic repair device to inhibit migration of the aortic repair device through the aorta and/or windsocking of the aortic repair device within the aorta caused by high flow and/or high pressure of blood flowing through the aorta. In additional aspects of the present technology, the leading tip capture mechanism can facilitate a staged deployment of different portions of the leading end portion of the aortic repair device to allow for more accurate and desirable positioning of the aortic repair device within the aorta.

In some embodiments, the inner catheter assembly additionally or alternatively includes a trailing tip capture mechanism configured to be releasably secured to a trailing end portion of the aortic repair device. The trailing tip capture mechanism can capture and retain all or a portion of the trailing end portion after the aortic repair device is deployed from the outer catheter. The trailing tip capture mechanism can be actuated by, for example, retracting one or more release wires of the delivery system to selectively release the trailing end portion of the aortic repair device. In some aspects of the present technology, during deployment of the aortic repair device, the trailing tip capture mechanism can tension the aortic repair device to inhibit the trailing end portion of the aortic repair device from migrating through the aorta as leading portions of the aortic repair device are sequentially deployed from the outer catheter.

In some embodiments, the inner catheter assembly and/or the outer catheter can be shaped and/or steerable to facilitate positioning of the aortic repair device within the aorta even where the delivery system traverses angled and/or tortuous anatomy. For example, the inner catheter assembly and/or the outer catheter can be shaped/steered to deploy the aortic repair device squarely within the aorta to, for example, inhibit or even prevent the aortic repair device from blocking/covering branching vessels (e.g., the left coronary artery) and to provide a long treatment region within the aorta.

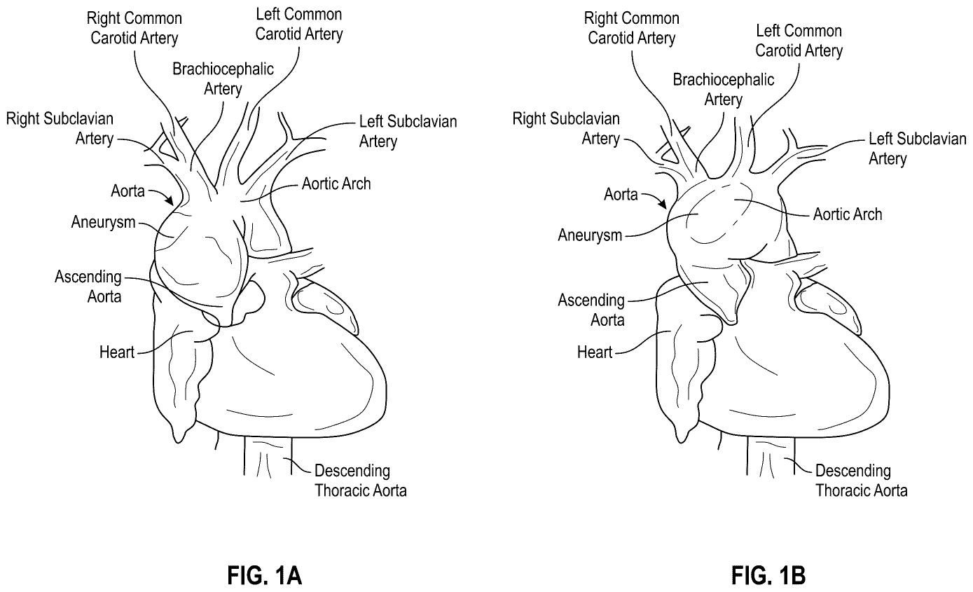

Specific details of several embodiments of the present technology are described herein with reference to FIGS. 1A-50B. The present technology, however, can be practiced without some of these specific details. In some instances, well-known structures and techniques often associated with catheter-based delivery systems, implantable repair devices, and the like, have not been shown in detail so as not to obscure the present technology. The terminology used in the description presented below is intended to be interpreted in its broadest reasonable manner, even though it is being used in conjunction with a detailed description of certain specific embodiments of the disclosure. Certain terms can even be emphasized below; however, any terminology intended to be interpreted in any restricted manner will be overtly and specifically defined as such in this Detailed Description section.

The accompanying Figures depict embodiments of the present technology and are not intended to be limiting of its scope. The sizes of various depicted elements are not necessarily drawn to scale, and these various elements can be arbitrarily enlarged to improve legibility. Component details can be abstracted in the Figures to exclude details such as position of components and certain precise connections between such components when such details are unnecessary for a complete understanding of how to make and use the present technology. Many of the details, dimensions, angles, and other features shown in the Figures are merely illustrative of particular embodiments of the disclosure. Accordingly, other embodiments can have other details, dimensions, angles, and features without departing from the spirit or scope of the present technology.

When used with reference to an aortic repair device, the term “distal” can reference a portion of the aortic repair device positioned and/or configured to be positioned farther from the heart and downstream in the path of blood flow, while the term “proximal” can reference a portion of the aortic repair device positioned and/or configured to be positioned closer to the heart and upstream in the path of blood flow. In contrast, when used with reference to a catheter subsystem or delivery procedure, the term “distal” can reference a portion of the catheter system farther from an operator and/or handle while the term “proximal” can reference a portion of the catheter system closer to the operator and/or handle. Accordingly, as set forth below, often a proximal portion of an aortic repair device is farther from the operator and/or handle of a catheter system used to deliver the aortic repair device than a distal portion of the aortic repair device. Likewise, often a distal portion of the aortic repair device is closer to the operator and/or handle of the catheter system than the proximal portion.

Also, as used herein, the designations “rearward,” “forward,” “upward,” “downward,” etc., are not meant to limit the referenced component to use in a specific orientation. It will be appreciated that such designations refer to the orientation of the referenced component as illustrated in the Figures; the systems of the present technology can be used in any orientation suitable to the user.

The headings provided herein are for convenience only and should not be construed as limiting the subject matter disclosed.

### I. OVERVIEW

FIGS. 1A and 1B are side views of a diseased aorta and surrounding anatomy in which an aortic repair device can be implanted by a delivery system in accordance with embodiments of the present technology. Referring to FIGS. 1A and 1B together, the aorta is the largest vessel in the human body and carries oxygenated blood away from the left ventricle and the aortic valve of the heart for circulation to all parts of the body. The aorta is divided into different segments including the ascending aorta (which extends from the left ventricle), the aortic arch, and the descending thoracic aorta. The ascending aorta and the aortic arch can together be referred to as the “proximal aorta.” The aorta includes branches into several supra-aortic arteries including the brachiocephalic artery, the left common carotid artery, and the left subclavian artery—each of which extends from the aortic arch. The brachiocephalic artery is the first branch of aortic arch and feeds blood flow to the right common carotid artery and the right subclavian artery for supply to the right arm, head, and neck. It is also known as the innominate artery or the brachiocephalic trunk. The left common carotid artery is the second branch of the aortic arch and feeds blood flow to the left head and neck. The left subclavian artery is the third branch of the aortic arch and feeds blood flow to the left arm.

The diseased aorta includes an aneurysm in the ascending aorta in FIG. 1A and an aneurysm in the aortic arch in FIG. 1B. Aortic aneurysms are enlargements (e.g., dilations) of the aorta that weaken the aorta and increase the likelihood of rupture. Most people with aortic aneurysms do not have symptoms until the aorta ruptures, and the mortality rate after an aortic aneurysm rupture can exceed 90%. Often, aortic aneurysms are detected during routine medical testing such as chest X-rays, computed tomography (CT) imaging, ultrasound imaging of the heart, magnetic resonance imaging (MRI), and the like.

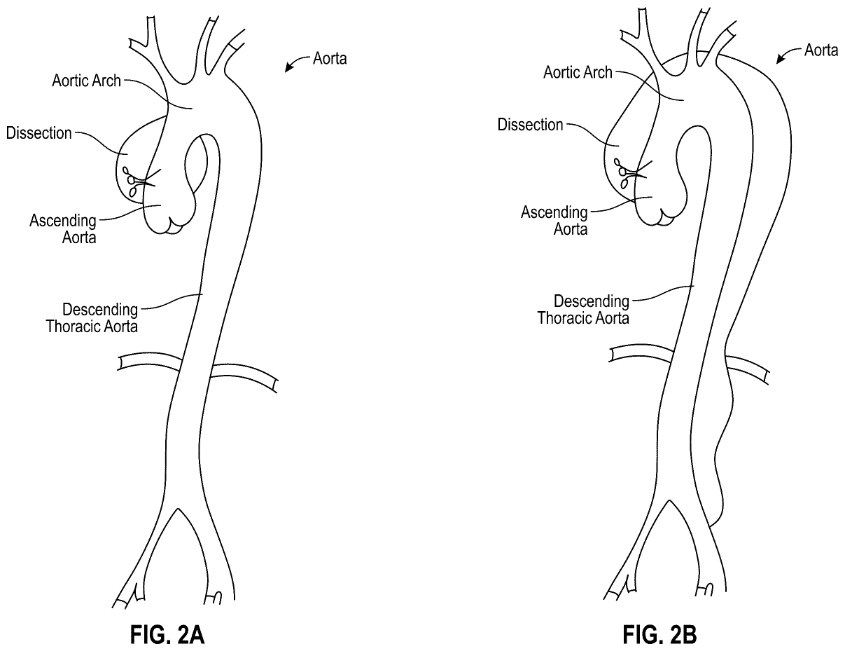

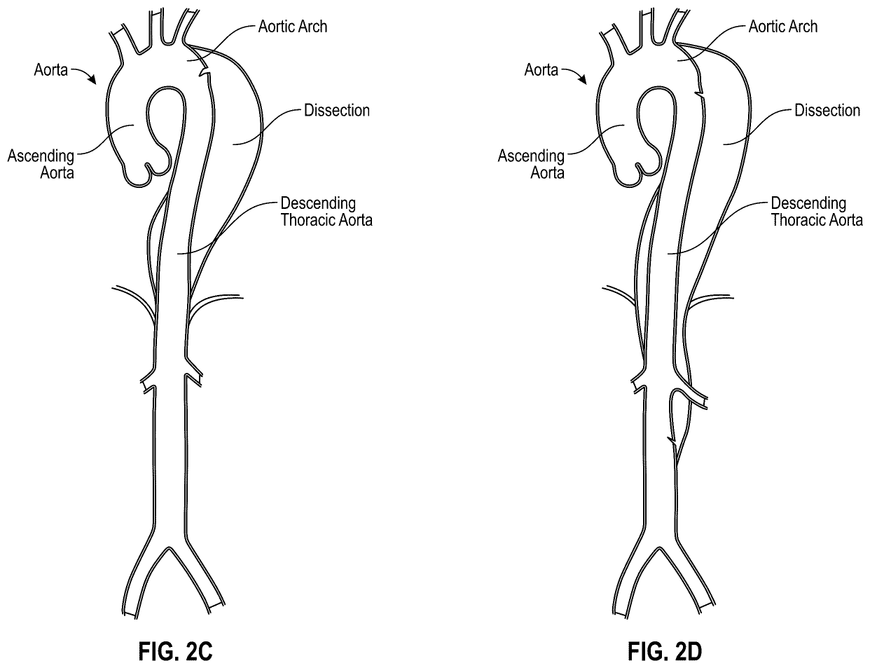

FIGS. 2A-D are side cross-sectional views of a diseased aorta in which an aortic repair device can be implanted by a delivery system in accordance with embodiments of the present technology. The diseased aorta includes DeBakey Type II, a smaller Type A aortic dissection contained within the ascending aorta, in FIG. 2A, and a DeBakey Type I, a larger Type A aortic dissection that extends beyond the ascending aorta, in FIG. 2B. The diseased aorta includes a smaller Type B aortic dissection in FIG. 2C, and a larger Type B aortic dissection in FIG. 2D. Aortic dissections are tears of the intimal layer of the aorta that cause blood to dissect the intimal layer and block flow. Type A aortic dissections originate in the ascending aorta and can progressively extend from the ascending aorta, along the aortic arch, and/or down along the descending aorta as shown in FIG. 2B. Type B aortic dissections originate distal to the branching of the brachiocephalic trunk and can progressively extend down along the descending aorta as shown in FIG. 2D. Aortic dissections can be acute or chronic, with acute aortic dissections frequently causing a sudden onset of severe pain in the chest, back, and/or abdomen. Most acute aortic dissections require emergency surgery, and present about a 1% risk of death every hour for the first two days after dissection-meaning that urgent diagnosis and surgery are critical for patient treatment.

Referring to FIGS. 1A-2D together, aspects of the present technology are directed to aortic repair devices that can be implanted within the aorta to treat an aortic aneurysm, a Type A aortic dissection, a Type B aortic dissection, and/or other types of diseased states. In some embodiments, an aortic repair device can span across the origin of an aneurysm or dissection and provide one or more flow conduits for diverting blood flow away from and/or past the diseased portion. In some embodiments, an aortic repair device can span between and provide one or more flow conduits for directing blood flow between the aorta and one or more branching arteries.

### II. SELECTED EMBODIMENTS OF DELIVERY SYSTEMS FOR IMPLANTING AORTIC REPAIR DEVICES

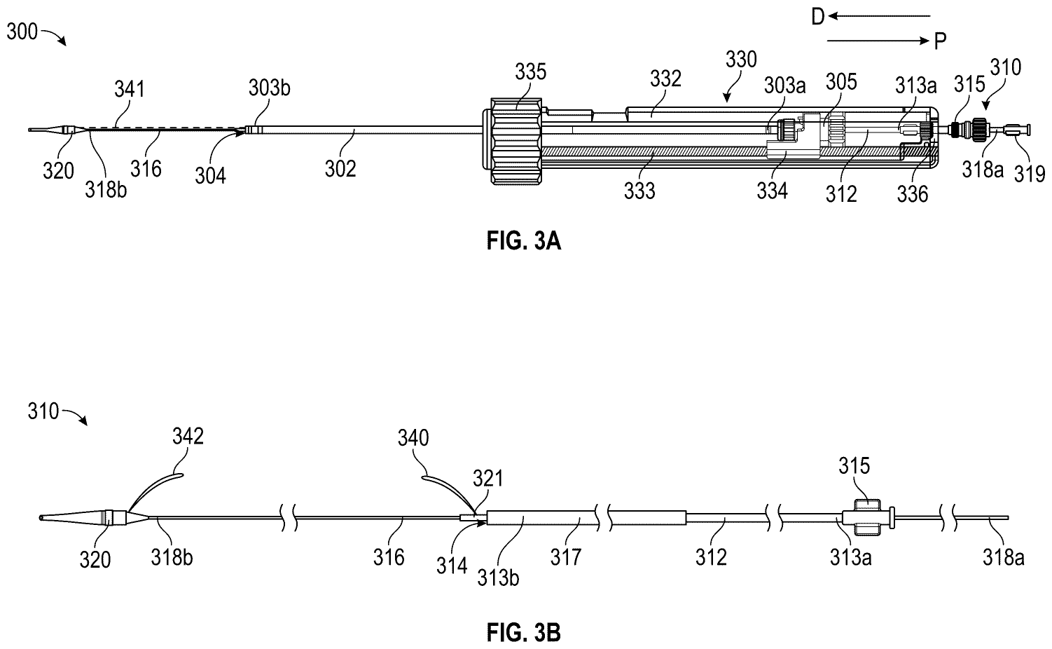

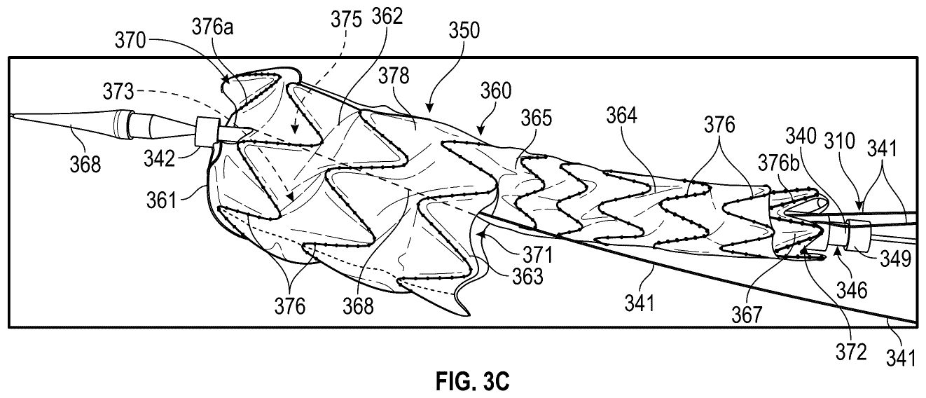

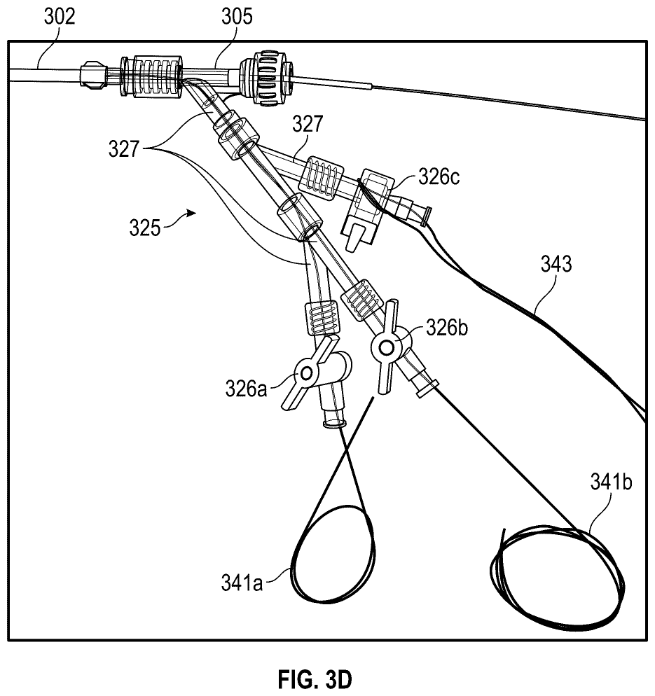

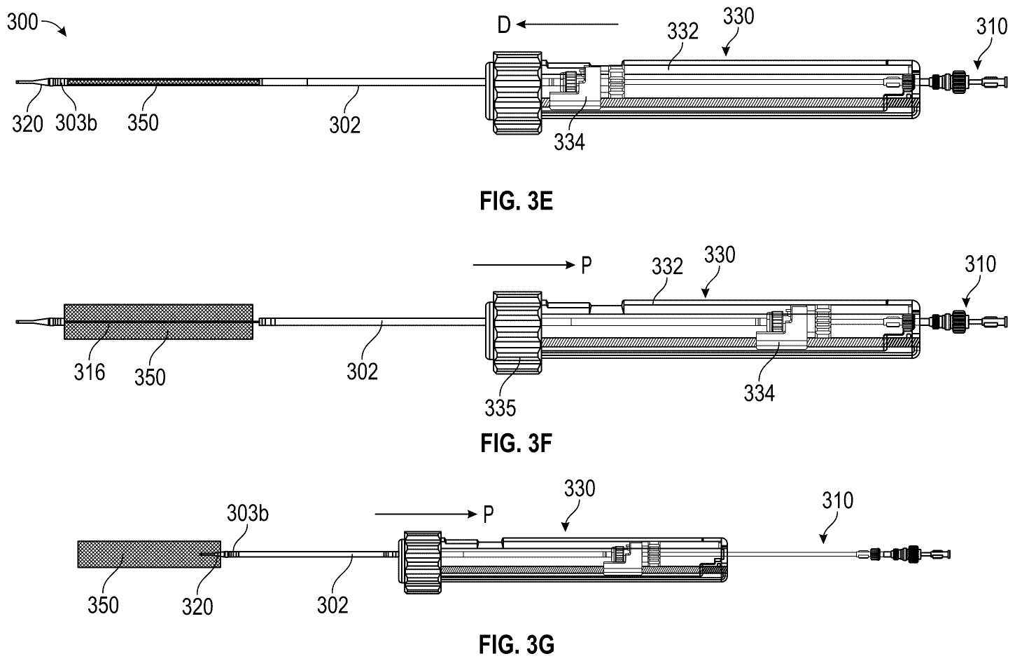

FIG. 3A is a side view of a delivery system 300 configured in accordance with embodiments of the present technology. In the illustrated embodiment, the delivery system 300 includes an outer catheter 302 (which can also be referred to as first catheter, an outer sheath, an elongated member, an outer tube, a delivery catheter, and/or the like), an inner catheter assembly 310 configured to extend at least partially through the outer catheter 302, and a handle 330 operably coupled to the outer catheter 302 and/or the inner catheter assembly 310. The outer catheter 302 includes a trailing (e.g., proximal) end portion 303a, a leading (e.g., distal) end portion 303b, and a lumen 304 extending between the trailing and leading end portions 303a-b. The trailing end portion 303a of the outer catheter 302 can be coupled to a first connector 305, such as a valve assembly or adapter (e.g., Tuohy-Borst adapter) including one or more fluid ports, fluid channels, hemostasis valves, and/or the like.

FIG. 3B is a side view of the inner catheter assembly 310 of the delivery system 300 in accordance with embodiments of the present technology. Referring to FIGS. 3A and 3B, the inner catheter assembly 310 extends at least partially through the lumen 304 of the outer catheter 302 and includes a pusher catheter 312 (which can also be referred to as a second catheter, a mid catheter, an elongated member, a mid tube, and/or the like) and an inner catheter 316 (which can also be referred to as third catheter, an inner shaft, an elongated member, an inner tube, and/or the like) extending at least partially through the pusher catheter 312. More specifically, the pusher catheter 312 includes a trailing (e.g., proximal) end portion 313a, a leading (e.g., distal) end portion 313b (FIG. 3B), and a lumen 314 (FIG. 3B) extending between the trailing and leading end portions 313a-b. The trailing end portion 313a of the pusher catheter 312 can be coupled to a second connector 315, such as a valve assembly or an adapter (e.g., Tuohy-Borst adapter) including one or more fluid ports, fluid channels, hemostasis valves, and/or the like. As shown in FIG. 3B, in some embodiments the pusher catheter 312 includes an enlarged portion 317 at a distal portion thereof, such as a tube having a wider diameter than a proximal portion of the pusher catheter 312. The pusher catheter 312 (e.g., the enlarged portion 317) can contact a trailing end portion of an aortic repair device secured to the inner catheter assembly 310 to help transmit pushing forces from the handle 330 to the aortic repair device and/or to compress the aortic repair device via movement of the pusher catheter 312 relative to the inner catheter 316.

In the illustrated embodiment, the inner catheter 316 extends entirely through the lumen 314 of the pusher catheter 312 such that a portion of the inner catheter 316 is positioned distal to the pusher catheter 312. The inner catheter 316 can include a trailing (e.g., proximal) end portion 318a, a leading (e.g., distal) end portion 318b (FIG. 3B), and a lumen (obscured in FIGS. 3A and 3B) extending between the trailing and leading end portions 318a-b. The trailing end portion 318a of the inner catheter 316 can be coupled to a third connector 319 (FIG. 3A), such as a valve assembly or an adapter (e.g., Tuohy-Borst adapter) including one or more fluid ports, fluid channels, hemostasis valves, and/or the like, and the leading end portion 318b of the inner catheter 316 can be coupled to a tip member 320. The lumen of the inner catheter 316 and the tip member 320 can receive a guidewire (not shown) therethrough. The delivery system 300 can be routed/advanced over the guidewire to a target location in and/or proximate to a diseased aorta.

Referring to FIG. 3B, in the illustrated embodiment the inner catheter assembly 310 can further include a stopper member 321 positioned at least partially between the pusher catheter 312 (e.g., the enlarged portion 317 thereof) and the leading end portion 318b of the inner catheter 316. The stopper member 321 can inhibit relative motion between the pusher catheter 312 and the inner catheter 316 and/or inhibit the inner catheter 316 from contacting (e.g., driving into) the pusher catheter 312 during manipulation of the delivery system 300.

Referring to FIG. 3A, the handle 330 can include a housing 332, a leadscrew 333 positioned at least partially within the housing 332, a carriage 334 operably (e.g., threadedly) coupled to the leadscrew 333 within the housing 332, and an actuator 335 operably coupled to the leadscrew 333 (e.g., via one or more gears; not shown). The first connector 305 can be coupled to (e.g., mounted to) the carriage 334 such that movement of the carriage 334 moves the first connector 305 and the outer catheter 302. The inner catheter assembly 310 can be releasably coupled to the housing 332. For example, a proximal portion 336 (e.g., a proximal wall) of the housing 332 can include a retaining feature such as a groove, quick-release assembly, and/or the like configured (e.g., shaped, sized, positioned) to retain the second connector 315 in axial position relative to the housing 332. The actuator 335 can be actuated to rotate the leadscrew 333 to thereby drive the carriage 334 and the outer catheter 302 proximally and/or distally relative to the housing 332 and the inner catheter assembly 310 secured thereto. For example, in the illustrated embodiment the actuator 335 is a rotatable knob that can be rotated in a first direction (e.g., a clockwise or counterclockwise direction) to drive the carriage 334 and the outer catheter 302 proximally in the direction of arrow P relative to the inner catheter assembly 310 and in a second direction (e.g., opposite the first direction) to drive the carriage 334 and the outer catheter 302 distally in the direction of arrow D relative to the inner catheter assembly 310.

An aortic repair device can be releasably coupled to the inner catheter assembly 310 and retained/deployed by the outer catheter 302. For example, the aortic repair device can be secured around the inner catheter 316 between the tip member 320 and the pusher catheter 312. More specifically, FIG. 3C is a perspective side view of an aortic repair device 350 (which can also be referred to as an aortic prosthesis, an aortic treatment device, an aortic implant, and/or the like) secured to/positioned around a distal portion of the inner catheter assembly 310 in accordance with embodiments of the present technology. The aortic repair device 350 can include some features that are at least generally similar in structure and function, or identical in structure and function, to any of the aortic repair devices disclosed in U.S. patent application Ser. No. 18/179,254, Filed Mar. 6, 2023, and titled “DEVICES FOR AORTIC REPAIR, AND ASSOCIATED SYSTEMS AND METHODS,” which is incorporated by reference herein in its entirety.

In the illustrated embodiment, the aortic repair device 350 comprises a base member 360 configured to be implanted in a diseased aorta. The base member 360 includes a tubular body 362 (e.g., a main body) and a tubular leg 364 extending distally from the body 362. More specifically, the body 362 includes a leading (e.g., first, proximal) end portion 361 defining a leading (e.g., first, proximal) terminus of the base member 360 and a trailing (e.g., second, distal) end portion 363. The leg 364 includes a leading (e.g., first, proximal) end portion 365 coupled to and/or integrally extending from the trailing end portion 363 of the body 362 and a trailing (e.g., second, distal) end portion 367 defining a leading (e.g., second, distal) terminus of the base member 360. The base member 360 can be generally hollow and define one or more lumens (e.g., flow conduits) therethrough. For example, the base member 360 can include (i) a leading body opening 370 (e.g., a fluid opening, a first opening, a first body fluid opening, an inlet, and/or the like) at the leading end portion 361 of the body 362, (ii) a trailing body opening 371 (e.g., a fluid opening, a second opening, a second body fluid opening, a distal body outlet, and/or the like) at the trailing end portion 363 of the body 362, and (iii) a trailing leg opening 372 (e.g., a fluid opening, a third opening, a leg fluid opening, a distal leg outlet, and/or the like) at the trailing end portion 367 of the leg 364. A diameter of the body 362 can be sized to generally match or be larger than (e.g., oversized relative to) the diameter of a patient's aorta (e.g., between about 20-60 millimeters, between about 26-54 millimeters, about 40 millimeters). A diameter of the leg 364 can be sized to generally match or be larger than a branch vessel of the patient's aorta, such as the brachiocephalic artery (e.g., between about 10-22 millimeters, about 16 millimeters).

In some embodiments, the base member 360 further includes a septum 368 (shown schematically in FIG. 3C; e.g., a flow divider) positioned within the body 362 and dividing the body 362 into a primary lumen 373 (e.g., a first lumen) and a branch or secondary lumen 375 (e.g., a second lumen). The septum 368 can extend entirely or partially through the body 362 from the trailing end portion 363 toward the leading end portion 361, and can be centered or offset within the body 362 such that the primary and secondary lumens 373, 375 have the same or different sizes. The primary lumen 373 can extend from and define a flow path (e.g., conduit) between the leading body opening 370 and the trailing body opening 371. Similarly, the secondary lumen 375 can extend from and define a flow path between the leading body opening 370 and the trailing leg opening 372.

In some embodiments, the base member 360 is an expandable stent graft comprising one or more stents 376 and a graft material 378. The stents 376 can comprise one or multiple interconnected struts and can also be referred to as a stent structure. The body 362 and the leg 364 can be integrally formed as part of the same stent graft, or can be separate components that are releasably or permanently coupled together. The graft material 378 can comprise fabric, woven polyester, polytetrafluoroethylene, polyurethane, silicone, and/or other suitable materials known in the art of stent grafts, and is configured to inhibit or even prevent blood flow therethrough. In some embodiments, the septum 368 comprises the same material as the graft material 378 or another type of graft material. Accordingly, the graft material 378 and the septum 368 can define and enclose the primary lumen 373 and the secondary lumen 375 and are configured to maintain blood flowing along the flow paths defined thereby.

The stents 376 can extend circumferentially to define the tubular shape of the body 362 and the leg 364 and can be interconnected or separate. In some embodiments, the stents 376 have the illustrated V-pattern shape (e.g., including alternating proximal and distal apices). The stents 376 can be coupled to an outer surface of the graft material 378 as shown in FIGS. 3A-3C via stitching and/or suitable techniques, and/or can be coupled to an inner surface of the graft material 378. The stents 376 can be configured to self-expand and, accordingly, can be formed from a shape memory material, such as nickel-titanium alloy (nitinol). In other embodiments, the shape of the stents 376, the number of the stents 376, and/or the arrangement of the stents 376 can be varied.

In the illustrated embodiment, the leading end portion 361 of the body 362 is positioned adjacent the tip member 320 of the inner catheter assembly 310. Referring to FIGS. 3B and 3C, the inner catheter assembly 310 includes (i) a leading tip capture mechanism 342 configured (e.g., shaped, sized, positioned) to releasably secure the leading end portion 361 of the aortic repair device 350 to the inner catheter assembly 310 and (ii) a trailing tip capture mechanism 340 configured to releasably secure the trailing end portion 367 of the aortic repair device 350 to the inner catheter assembly 310. The leading tip capture mechanism 342 can be coupled to the tip member 320, and the trailing tip capture mechanism 340 can be coupled to the inner catheter 316 and/or the stopper member 321.

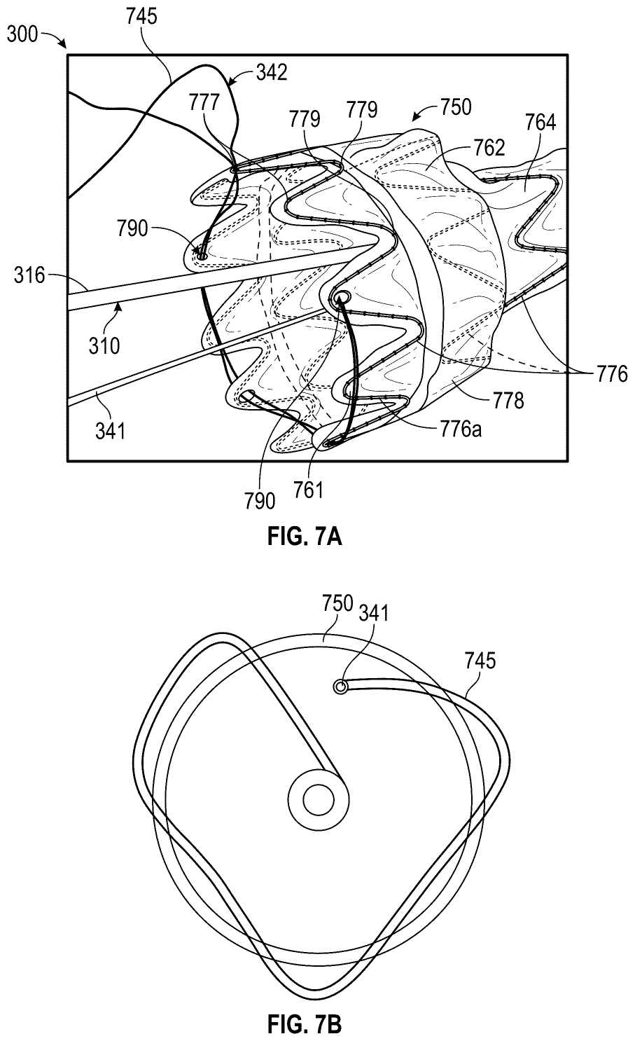

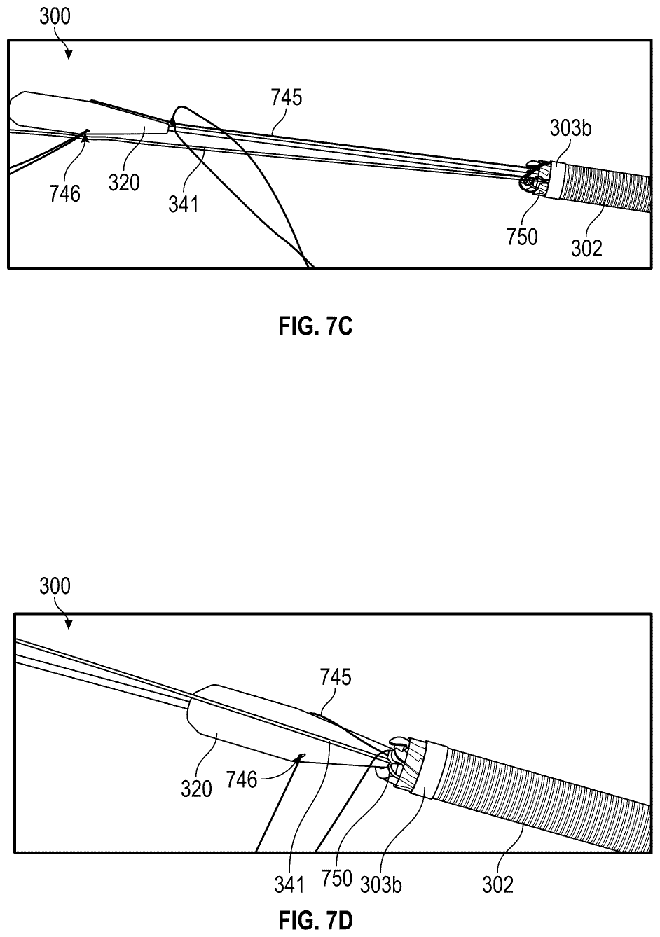

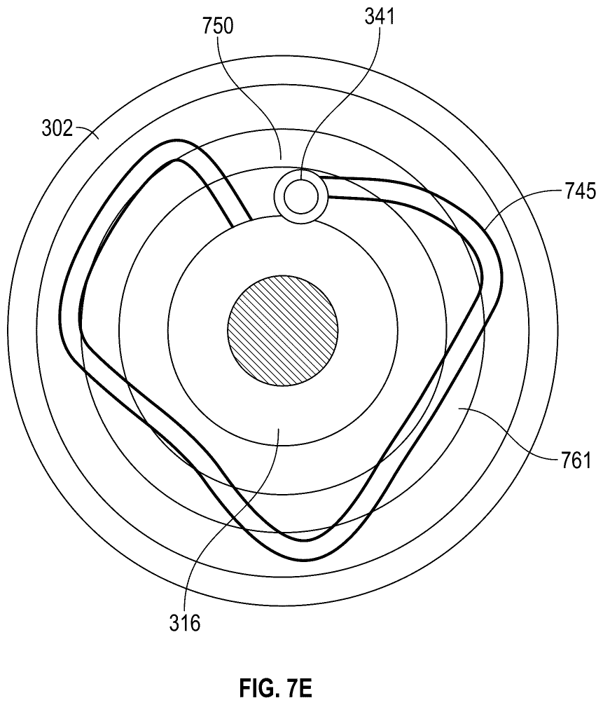

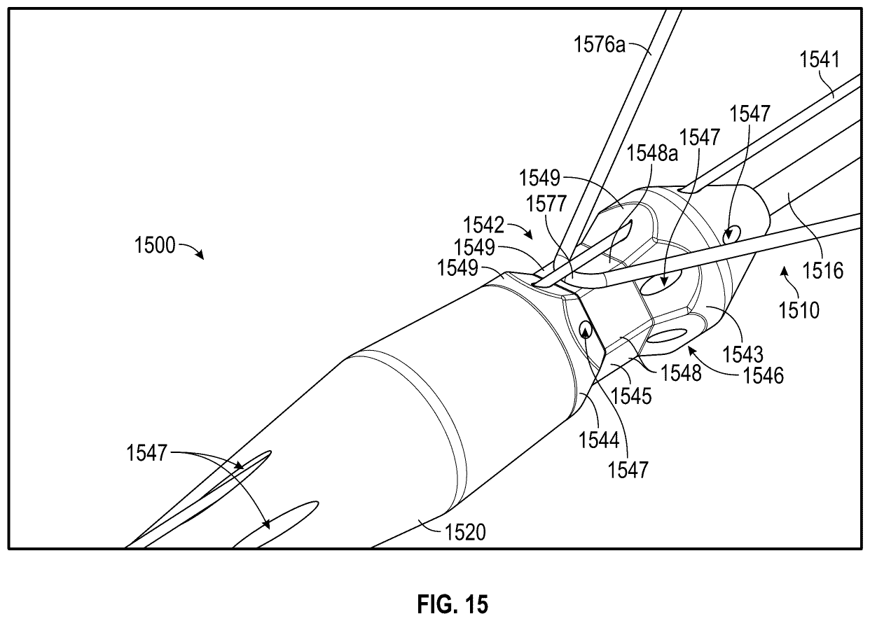

In the embodiment illustrated in FIG. 3B, the leading and trailing tip capture mechanisms 342, 340 comprise loops of thin, elongated material (e.g., suture loops) that can be secured to the aortic repair device 350 via a release wire 341 (FIG. 3A) that engages the leading and trailing tip capture mechanisms 342, 340 in a secured position, and that can be retracted proximally to release the leading and trailing tip capture mechanisms 342, 340 from the aortic repair device 350 to release the aortic repair device 350 from the inner catheter assembly 310, as described in greater detail below with reference to FIGS. 7A-14. Referring to FIG. 3A, the release wire 341 can be routed proximally through the outer catheter 302 and/or the pusher catheter 312 for access by a user (e.g., via the first connector 305 and/or the second connector 315). In the embodiment illustrated in FIG. 3C, the leading and trailing tip capture mechanisms 342, 340 comprise mechanical connectors that can be releasably secured to, for example, a leading one of the stents 376a and a trailing one of the stents 376b, respectively, via one or more of the release wires 341, as described in greater detail below with reference to FIGS. 15-17D.

Referring to FIGS. 3A-3C, the delivery system 300 can include multiple of the release wires 341 that can be individually actuated (e.g., pulled proximally) to sequentially/selectively release the aortic repair device 350 from the inner catheter assembly 310, one or more tethers for adjusting an orientation of the aortic repair device 350, and/or one or more additional components for facilitating deployment of the aortic repair device 350. Accordingly, in some embodiments the delivery system 300 includes one or more additional connectors coupled to the lumen 304 of the outer catheter 302 and/or the lumen 314 of the pusher catheter 312 for receiving corresponding ones of the release wires, tethers, and/or other components. FIG. 3D, for example, is a perspective side view of a connector assembly 325 that can be incorporated into the delivery system 300 in accordance with embodiments of the present technology. In the illustrated embodiment, the connector assembly 325 is coupled to the lumen 314 of the pusher catheter 312 (FIG. 3B) via the second connector 315 but, in other embodiments, can be coupled to the lumen 304 (FIG. 3A) of the outer catheter 302 via the first connector 305. The connector assembly 325 can include multiple valves 326 (including individually first through third valves 326a-c, respectively) that branch from the first connector 305 via tubing 327. In the illustrated embodiment, the first valve 326a receives a first release wire 341a therethrough, the second valve 326b receives a second release wire 341b therethrough, and the third valve 326c receives a tether 343 therethrough. The first and second release wires 341a-b and the tether 343 can be pulled proximally through the first through third valves 326a-c, respectively, to actuate/release the aortic repair device 350 as described in further detail below with reference to FIGS. 7A-23 and 32A-34E. In some embodiments, the connector assembly 325 includes more or fewer of the valves 326, and/or individual ones of the valves 326 can receive multiple ones of the release wires 341 and/or the tethers 343 therethrough.

Referring to FIG. 3A, in the illustrated embodiment the delivery system 300 is in a deployed position in which the actuator 335 has been actuated to drive the carriage 334 proximally within the housing 332 to retract the outer catheter 302 relative to the inner catheter assembly 310. FIGS. 3E-3G are side views of the delivery system 300 in a delivery position (e.g., an undeployed position), the deployed position, and a retracted position, respectively, in accordance with embodiments of the present technology.

Referring to FIG. 3E, in the delivery position, the carriage 334 is positioned distally within the housing 332 of the handle 330 such that the outer catheter 302 is advanced distally over the inner catheter assembly 310. In some embodiments, the leading end portion 303b of the outer catheter 302 overlaps and/or at least partially engages the tip member 320 in the delivery position. The outer catheter 302 can extend over and radially compress/collapse the aortic repair device 350 (shown schematically) within the lumen 334 (FIG. 3A) of the outer catheter 302 in the delivery position. The delivery system 300 can be intravascularly advanced through the vasculature of a patient in the delivery position to, at, within, and/or proximate a diseased aorta via any suitable intravascular path—such as an aortic approach, a transfemoral approach, a transcarotid approach, a transsubclavian approach, a transapical approach, and so on. More specifically, the handle 330 can be moved distally as in the direction of the arrow D to drive the inner catheter assembly 310 and the outer catheter 302 along the intravascular path. During advancement, the inner catheter assembly 310 and the outer catheter 302 are secured to the handle 330 such that these components move together without relative motion therebetween. Additionally, the tip member 320 can be atraumatic to inhibit or even prevent damage to the vasculature during advancement.

Referring to FIG. 3F, when the delivery system 300 is positioned at a target implantation (e.g., deployment) position within the vasculature, the delivery system 300 can be moved to the deployed position by actuating (e.g., rotating) the actuator 335 to drive the carriage 334 proximally in the direction of the arrow P within the housing 332 of the handle 330 to retract the outer catheter 302 relative to the inner catheter assembly 310. The retraction allows the aortic repair device 350 (shown schematically) to expand away from the inner catheter 316.

Referring to FIG. 3G, after releasing the aortic repair device 350 from the inner catheter assembly 310 (e.g., by actuating the leading and/or trailing tip capture mechanisms 342, 340 shown in FIGS. 3A and 3B), the delivery system 300 can be moved to the retracted position by (i) releasing (e.g., unlocking, disengaging) the inner catheter assembly 310 from the handle 330 and (ii) pulling the inner catheter assembly 310 proximally in the direction of the arrow P to retract the inner catheter assembly 310 relative to the outer catheter 302. In some embodiments, the leading end portion 303b of the outer catheter 302 overlaps and/or at least partially engages the tip member 320 in the retracted position. The delivery system 300 can be intravascularly withdrawn from the patient along the intravascular path in the retracted position. More specifically, the handle 330 and the inner catheter assembly 310 can be retracted proximally in the direction of the arrow P to withdraw these components along the intravascular path.

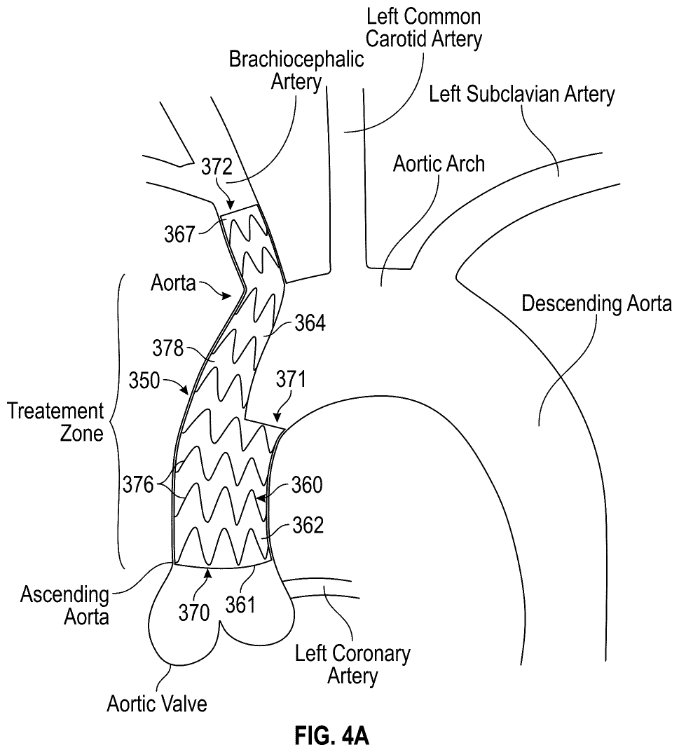

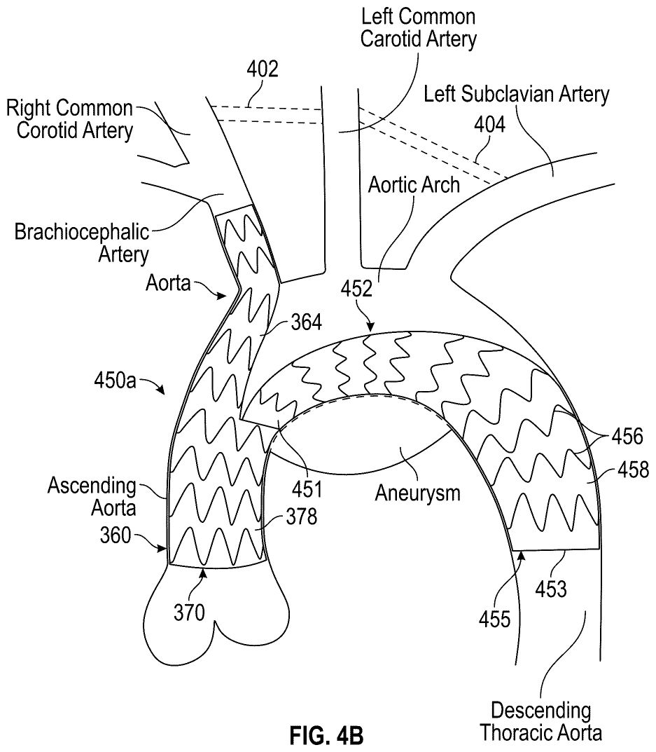

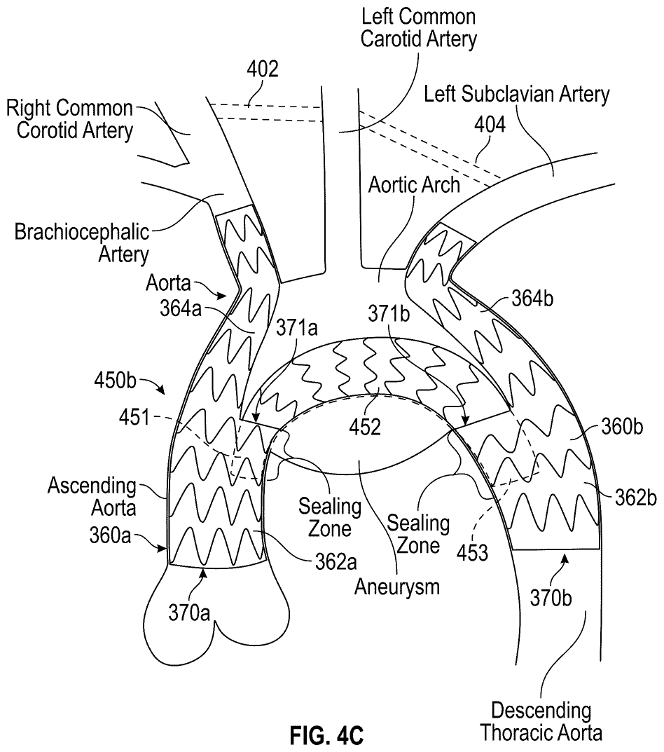

FIG. 4A is a side view of the aortic repair device 350 of FIG. 3C implanted within an aorta after implantation via the delivery system 300 of FIG. 3A in accordance with embodiments of the present technology. In some embodiments, the aorta can include an aneurysm, dissection, and/or other diseased portion as described in detail above with reference to FIGS. 1A-2B. In the illustrated embodiment, the body 362 is implanted within the proximal aorta (e.g., the ascending aorta and/or the aortic arch) with the leading end portion 361 positioned proximate to the aortic valve, and the leg 364 extends from the body 362 to the brachiocephalic artery where the trialing end portion 367 is positioned. The stents 376 can expand the graft material 378 into contact with the inner wall of the aorta and/or the brachiocephalic artery to provide a seal between the vessel and the base member 360. More particularly, referring to FIGS. 3C and 4A, the body 362 can sealingly contact the inner wall of the proximal aorta such that all or substantially all blood flow through the aorta enters the proximal opening 370 and flows through either the primary lumen 373 or the secondary lumen 375. The base member 360 can direct the blood flow (i) through the primary lumen 373 and out of the body opening 371 into the aorta to perfuse the aorta and (ii) through the secondary lumen 375 and out of the leg opening 372 into the brachiocephalic artery to perfuse the brachiocephalic artery. In some aspects of the present technology, the base member 360 can be positioned against/adjacent to a diseased portion of the aorta, such as the origin of a dissection and/or aneurysm, to block blood flow into the diseased portion by diverting the blood flow through the base member 360 and past the diseased portion. The base member 360 can be delivered to the aorta in the collapsed configuration within the delivery system 300 (FIG. 3E), deployed from the outer catheter 302 (FIG. 3F), and released from the delivery system 300 (FIG. 3G) to have the position illustrated in FIG. 4A. In some embodiments the base member 360 can be implanted within the descending aorta in a reversed or flipped orientation.

FIG. 4B is a side view of an aortic repair device 450a implanted within an aorta in accordance with embodiments of the present technology. In the illustrated embodiment, the aortic repair device 450a includes the base member 360 described in detail above with reference to FIGS. 3C-4A, and a separate spanning member 452 coupled to (e.g., attached to, docked to) the base member 360. The spanning member 452 can have a tubular shape defining a lumen and can include features generally similar to those of the base member 360. For example, in some embodiments the spanning member 452 is an expandable stent graft comprising one or more struts or stents 456 and a graft material 458 coupled to the stents 456.

In the illustrated embodiment, the spanning member 452 includes a leading end portion 451 (e.g., a first end portion, a proximal end portion; partially obscured by the base member 360) defining a proximal terminus of the spanning member 452 and a trailing end portion 453 (e.g., a second end portion, a distal end portion) defining a distal terminus of the spanning member 452. The spanning member 452 can be generally hollow and define one or more lumens (e.g., flow conduits) therethrough. Accordingly, in the illustrated embodiment the spanning member 452 includes a leading opening (e.g., a fluid opening, a first spanning fluid opening, a leading spanning fluid opening, and/or the like; obscured by the base member 360) and a trailing opening 455 (e.g., a fluid opening, a second spanning fluid opening, a trailing spanning fluid opening, and/or the like).

Referring to FIGS. 3C and 4B, the proximal end portion 451 of the spanning member 452 can be at least partially positioned within the body 362 within the primary lumen 373 and can sealingly engage the body 362 within the primary lumen 373 to define a continuous blood flow path from the proximal opening 370 of the base member 360, through the primary lumen 373, and through the lumen of the spanning member 452 to the trailing opening 455. That is, the graft material 458 of the spanning member 452 can sealingly engage the septum 368 and the graft material 378 of the base member 360 within the primary lumen 373 such that blood flow is routed through the primary lumen 373 to the lumen of the spanning member 452. In some embodiments, at least a portion of the spanning member 452 (e.g., the trailing end portion 453) sealingly engages the aorta within the aortic arch and/or the descending thoracic aorta. Accordingly, the aortic repair device 450a can direct blood flow through the secondary lumen 375 to the brachiocephalic artery and through the primary lumen 373 and the spanning member 452 to the descending thoracic aorta. Accordingly, in some aspects of the present technology the aortic repair device 450a can divert blood flow past a diseased portion of the aorta, such an aneurysm in the aortic arch shown in FIG. 4B.

The spanning member 452 can be delivered to the aorta in a collapsed configuration within the delivery system 300, or a separate similar or identical delivery system, in the same or a separate procedure as the base member 360. For example, referring to FIGS. 3A-3G and 4B, the leading end portion 451 of the spanning member 452 can be coupled to the inner catheter assembly 310 via the leading tip capture mechanism 342, and the trailing end portion 453 of the spanning member 452 can be coupled to the inner catheter assembly 310 via the trailing tip capture mechanism 340. The spanning member 452 can be delivered to the aorta in the delivery position (FIG. 3E) with the spanning member 452 collapsed within the outer catheter 302 and can then be deployed from the outer catheter 302 (FIG. 3F) and released from the delivery system 300 (FIG. 3G) to have the position illustrated in FIG. 4B.

Often a patient may initially only need treatment within the ascending aorta and/or the aortic arch to treat an initial dissection or aneurysm but, at a later time (e.g., months or years later), may require additional treatment of the aortic arch and/or the descending thoracic aorta as the diseased state progresses. Accordingly, the base member 360 can be implanted via the delivery system 300 during an initial procedure and the spanning member 452 can be implanted via the same or a separate delivery system 300 during a later procedure and modularly coupled to the base member 360 to provide further treatment of the aortic arch and/or the descending thoracic aorta (e.g., by bypassing blood flow past the aneurysm shown in FIG. 4B).

Referring to FIG. 4B, the spanning member 452 bypasses the left common carotid artery and the left subclavian artery and thus may partially or fully occlude those vessels. In some embodiments, a bypass 402 between the perfused right common carotid artery and/or a bypass 404 between the left common carotid artery and the left subclavian artery (and/or between the brachiocephalic artery and the left subclavian artery) can be surgically created to perfuse those vessels. In other embodiments, the aortic repair device 450a can include additional implantable devices (e.g., stent grafts) coupled to the spanning member 452 and/or the base member 360 that are configured (e.g., sized, shaped, positioned) to perfuse different branch vessels. Such other implantable devices can be delivered via the delivery system 300 (FIGS. 3A-3G) in the same or a similar manner as the base member 360 and the spanning member 452, or using a different delivery system. In other embodiments described in detail below, the aortic repair device 450a can include additional implantable devices (e.g., stent grafts) coupled to the spanning member 452 and/or the base member 360 that are configured (e.g., sized, shaped, positioned) to perfuse different branch vessels.

FIG. 4C is a side view of an aortic repair device 450b implanted within an aorta in accordance with embodiments of the present technology. In the illustrated embodiment, the aortic repair device 450b includes (i) the base member 360 described in detail above with reference to FIG. 3C (identified as first base member 360a) and (ii) the spanning member 452 coupled to (e.g., attached to, docked to) the first base member 360a as described in detail above with reference to FIG. 4B. In the illustrated embodiment, the aortic repair device 450b further includes a second base member 360b implanted at least partially within the descending thoracic aorta. The second base member 360b can be identical or substantially identical to the first base member 360a. For example, in the illustrated embodiment (i) the first base member 360a includes a first body 362a defining a leading body opening 370a and a trailing body opening 371a and (ii) the second base member 360b includes a second body 362b defining a leading body opening 371b and a trailing body opening 370b. While the first and second base members 362a-b can be identical or substantially identical, because the orientation of the first and second base members 362a-b is reversed in FIG. 4C, the leading body opening 370a and the trailing body opening 371a of the first base member 362a are equivalent in structure to the trailing body opening 370b and the leading body opening 371a, respectively, of the second base member 362b.

The spanning member 452 spans from the base member 360a and is coupled to the second base member 360b. Specifically, with reference to FIGS. 3C and 4A-4C together, the trailing end portion 453 of the spanning member 452 can be positioned within the second body 362b within the primary lumen 373 of the second base member 360b and can sealingly engage the second body 362b within the primary lumen 373 to define a continuous blood flow path from the spanning member 452 through the primary lumen 373 of the second body 362b, and out of the trailing (e.g., distal) body opening 370b. The leg 364b of the second base member 360b can extend into a second branch artery (e.g., the left subclavian artery, left carotid) for perfusing the second branch artery. For example, the second base member 360b can receive retrograde blood flow through the trailing body opening 370b for perfusing the second branch artery. Alternatively or additionally, the secondary lumen 375 of the second base member 360b can be perfused via the primary lumen 373 where the septum 368 extends only partially from the leading end portion 361 toward the trailing end portion 363. That is, blood flow from the spanning member 452 can flow into the primary lumen 373 within the second body 362b and around the septum 368 into the secondary lumen 375 where the septum 368 terminates within the second body 362b. In some aspects of the present technology, the aortic repair device 450b can provide for a full arch treatment in which (i) the leg 364a of the first base member 360a directs blood flow to a first branch artery (e.g., the brachiocephalic artery), (ii) the leg 364b of the second base member 360b directs blood flow to a second branch artery (e.g., the left subclavian artery), (iii) a third branch artery (e.g., the left common carotid artery) is perfused via bypass 402 and/or 404, and (iv) the primary lumens 373 of the first and second base members 360a-b and the spanning member 452 collectively direct blood flow to the descending thoracic aorta.

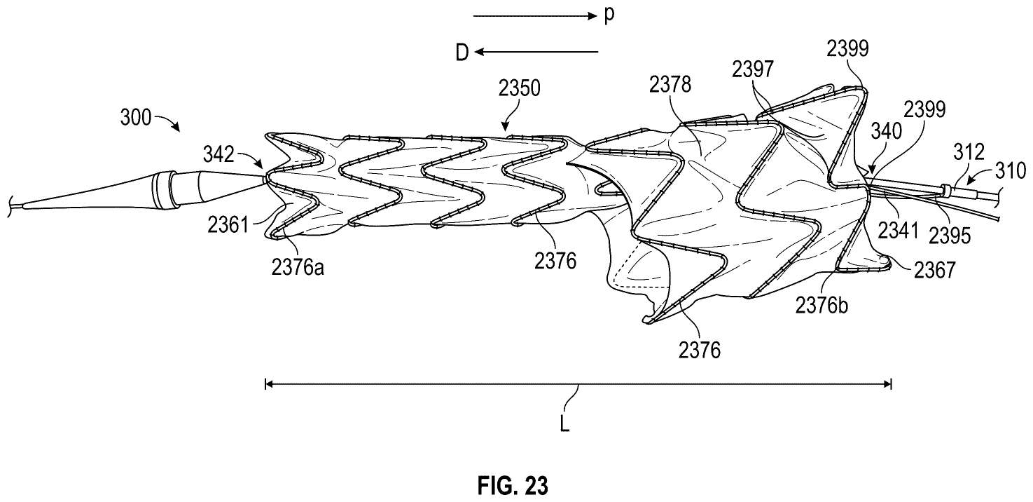

The second base member 360b can be delivered to the aorta in a collapsed configuration within the delivery system 300, or a separate similar or identical delivery system, in the same or a separate procedure as the first base member 360a and/or the spanning member 452. However, depending on the vascular path used to access the descending aorta, the second base member 360b can be mounted to the inner catheter assembly 310 in a reversed orientation—that is, with the end portion 361 coupled to the trailing tip capture mechanism 340 and the end portion 367 coupled to the leading tip capture mechanism 342 (e.g., as shown in FIG. 23).

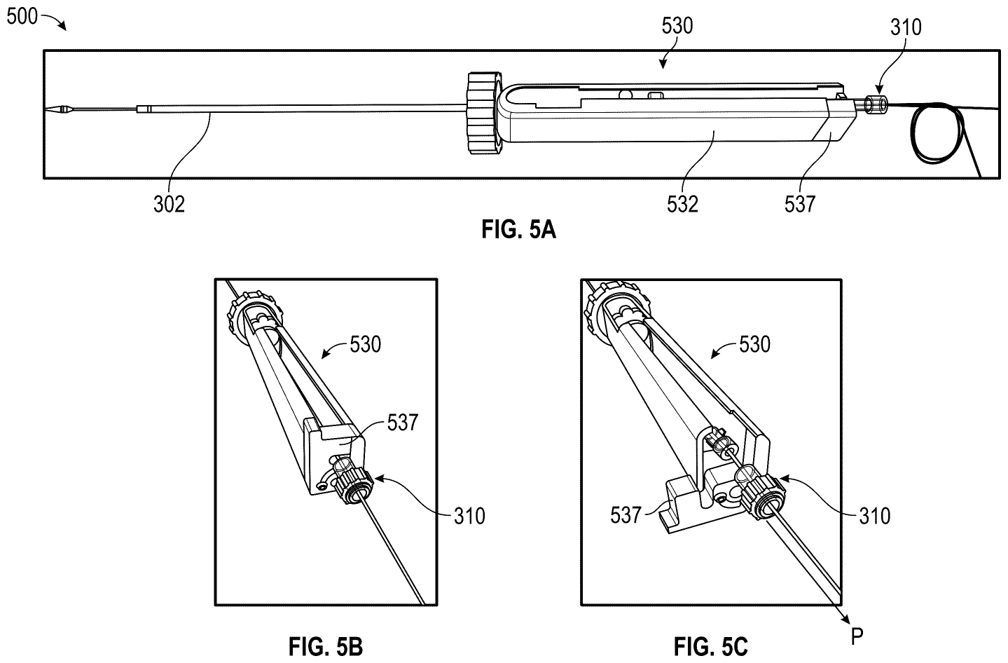

FIG. 5A is a perspective top view of a delivery system 500 in accordance with additional embodiments of the present technology. The delivery system 500 can include some features that are at least generally similar in structure and function, or identical in structure and function, to the corresponding features of the delivery system 300 described in detail above with reference to FIGS. 3A-3G and can operate in a generally similar or identical manner to the delivery system 300. For example, the delivery system 500 includes the outer catheter 302, the inner catheter assembly 310, and a handle 530 operably coupled to the outer catheter 302 for retracting/advancing the outer catheter 302 relative to the inner catheter assembly 310. In the illustrated embodiment, the handle 530 includes a housing 532 having a release member 537 pivotably coupled to a proximal portion thereof.

FIGS. 5B and 5C are enlarged perspective views of the handle 530 of the delivery system 500 of FIG. 5A in accordance with embodiments of the present technology. Referring to FIGS. 5B and 5C, the release member 537 is pivotable between (i) a locked position shown in FIG. 5B in which the inner catheter assembly 310 is unable to move axially past the release member 537 and (ii) an unlocked position shown in FIG. 5C in which the inner catheter assembly 310 can be moved axially (e.g., proximally) past the release member 537. Accordingly, referring to FIGS. 5A-5C, the delivery system 500 can be moved between a deployed position (e.g., as shown in FIG. 3F) and a retracted position (e.g., as shown in FIG. 3G) by moving the release member 537 from the locked position (FIG. 5B) to the unlocked position (FIG. 5C) and then pulling the inner catheter assembly 310 proximally in the direction of arrow P (FIG. 5C) to retract the inner catheter assembly 310 relative to the outer catheter 302. In some aspects of the present technology, the release member 537 can be moved to unlock the inner catheter assembly 310 from the handle 530 with little or no movement of the inner catheter assembly 310, such as a radial deflection (e.g., bend, pop) out of a mating groove of the housing 532. Accordingly, when the release member 537 is unlocked as shown in FIG. 5C, the inner catheter assembly 310 can be pulled straight backward proximally to move the delivery system 500 to the retracted position.

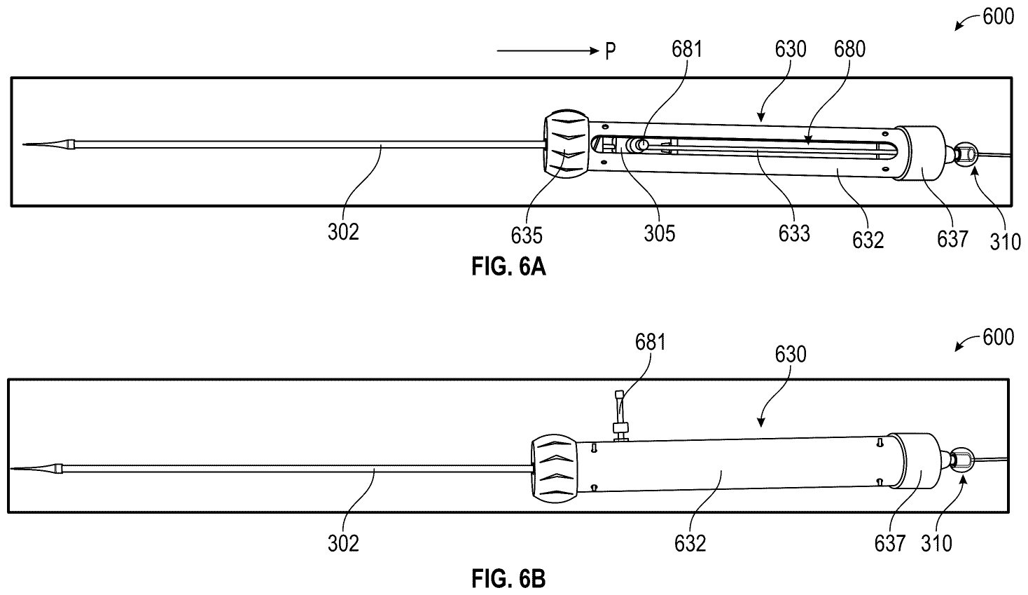

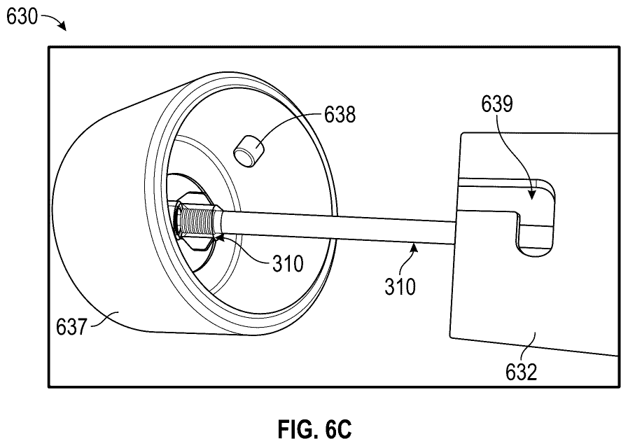

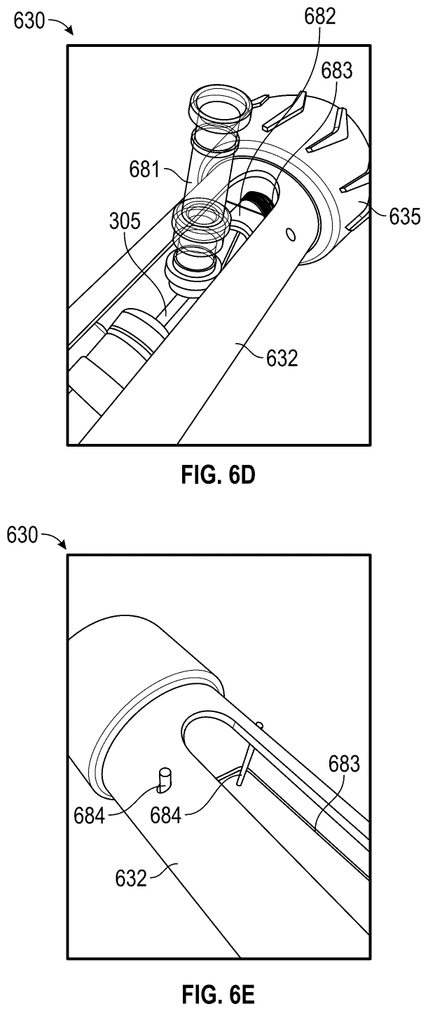

FIGS. 6A and 6B are a perspective top view and a perspective side view, respectively, of a delivery system 600 in a delivery position in accordance with additional embodiments of the present technology. The delivery system 600 can include some features that are at least generally similar in structure and function, or identical in structure and function, to the corresponding features of the delivery system 300 and/or the delivery system 500 described in detail above with reference to FIGS. 3A-3G and 5A-5C, and can operate in a generally similar or identical manner to the delivery system 300 and/or the delivery system 500. For example, the delivery system 600 includes the outer catheter 302, the inner catheter assembly 310, and a handle 630 operably coupled to the outer catheter 302 for retracting/advancing the outer catheter 302 relative to the inner catheter assembly 310.

In the illustrated embodiment, the handle 630 includes a housing 632 having a release member 637 (e.g., an endcap) releasably coupled to a proximal portion thereof. The release member 637 can be coupled to the inner catheter assembly 310 and is shown in a locked position in FIGS. 6A and 6B in which the release member 637 is secured to the housing 632 such that movement of the handle 630 moves the inner catheter assembly 310. FIG. 6C is an enlarged perspective view of the handle 630 of FIGS. 6A and 6B in accordance with embodiments of the present technology. In the illustrated embodiment, the release member 637 is in an unlocked position decoupled from the housing 632. The release member 637 can be a quick-release connector that includes one or more engagement features 638 (e.g., protrusions) that can be secured to/within one or more corresponding grooves 639 in the housing 632 in the locked position. The release member 637 can be rotated and pulled proximally away from the housing 632 to remove the engagement feature 638 from the groove 639 to decouple the release member 637 from the housing 632 and move the release member 637 to the unlocked position. Accordingly, referring to FIGS. 6A-6C, the delivery system 600 can be moved between a deployed position (e.g., as shown in FIG. 3F) and a retracted position (e.g., as shown in FIG. 3G) by moving the release member 637 from the locked position (FIGS. 6A and 6B) to the unlocked position (FIG. 6C) and then pulling the release member 637 and/or the inner catheter assembly 310 coupled thereto proximally to retract the inner catheter assembly 310 relative to the outer catheter 302. In some aspects of the present technology, the release member 637 can be moved to unlock the inner catheter assembly 310 from the handle 630 with little or no movement of the inner catheter assembly 310, such as a radial deflection (e.g., bend, pop) out of a mating groove of the housing 632. Accordingly, when the release member 637 is unlocked as shown in FIG. 6C, the inner catheter assembly 310 can be pulled straight backward proximally to move the delivery system 600 to the retracted position.

Referring to FIG. 6A, the handle 630 further includes a track 633 positioned within the housing 632 and an actuator 635. In the illustrated embodiment, the first connector 305 is slidably positioned along the track 633. In other embodiments, the first connector 305 can be coupled to (e.g., mounted to) a separate carriage (e.g., the carriage 334 of FIGS. 3A and 3B) such that movement of the carriage moves the first connector 305 and the outer catheter 302. The housing 632 can define a slot 680 extending above the track 633, and the first connector 305 can be coupled to a slider 681 extending through the slot 680 (also shown in FIG. 6B) from within the housing 632 such that the slider 681 is accessible to a user.

FIGS. 6D and 6E are perspective views of a distal portion and a proximal portion, respectively, of the handle 630 of FIGS. 6A and 6B in accordance with embodiments of the present technology. Referring to FIG. 6D, the actuator 635 is operably coupled to a wheel or spool 682 positioned within the housing 632. Referring to FIGS. 6D and 6E, in the illustrated embodiment a cable 683 is at least partially wound around the spool 682 and extends proximally through the housing 632, around one or more posts 684 (FIG. 6E) secured to the proximal portion of the handle 630, and back distally to the first connector 305. Referring to FIGS. 6A, 6B, 6D, and 6E, the actuator 635 can be actuated (e.g., rotated) to rotate the spool 682 to uptake the cable 683 (e.g., to further wind the cable 683 around the spool 682) to thereby draw the first connector 305 and the outer catheter 302 proximally along the track 633 within the housing 632 to retract the outer catheter 302 relative to the inner catheter assembly 310. In some embodiments, additionally or alternatively to actuating the actuator 635, a user can grip the slider 681 and drive the slider 681 proximally in the direction of arrow P in FIG. 6A to drive the first connector 305 and the outer catheter 302 along the track 633. In some aspects of the present technology, retracting the outer catheter 302 via the slider 681 can be faster than retracting the outer catheter 302 via the actuator 635. Accordingly, the slider 681 can provide an alternative retraction mechanism that can override the actuator 635 for faster retraction of the outer catheter 302. In some embodiments, the track 633 is not threaded or otherwise engaged with the first connector 305 in a non-slidable arrangement to permit the slider 681 to freely drive the first connector 305 along the track 633.

### III. SELECTED EMBODIMENTS OF TIP CAPTURE MECHANISMS FOR DELIVERY SYSTEMS FOR IMPLANTING AORTIC REPAIR DEVICES

FIGS. 7A-23, 41A-43D, and 45A-45C illustrate various embodiments of tip capture mechanisms of a delivery system for releasably securing a leading and/or trailing end portion of an aortic repair device in accordance with embodiments of the present technology. The various tip capture mechanisms can be incorporated into and used in any of the delivery systems described herein (e.g., as the leading tip capture mechanism 342 and/or the trailing tip capture mechanism 340 of the delivery system 300 of FIGS. 3A-3G). Moreover, the various aortic repair devices described with reference to FIGS. 7A-23, 41A-43D, and 45A-45C can include some features that are at least generally similar in structure and function, or identical in structure and function, to one another and/or to the corresponding features of any of the aortic repair devices described in detail above with reference to FIGS. 3C and 4A-4C, and can operate in a generally similar or identical manner to one another and/or to the aortic repair devices of FIGS. 3C and 4A-4C. Moreover, one or more of the different features of the tip capture mechanisms of the present technology can be combined and/or omitted.

FIG. 7A is a perspective view of a distal portion of the delivery system 300 of FIGS. 3A-3G partially secured to an aortic repair device 750 in accordance with embodiments of the present technology. The aortic repair device 750 can be a base member (e.g., similar to the base member 360 of FIGS. 3C and 4A-4C) configured to be implanted in the ascending aorta or the descending aorta of a patient, or can be a spanning member (e.g., the spanning member 452 of FIGS. 4B and 4C) configured to be docked to a base member and to span the aortic arch of the patient. In the illustrated embodiment, the aortic repair device 750 is an expandable stent graft comprising one or more stents 776 coupled to an inner surface and/or outer surface of a graft material 778. The aortic repair device 750 can include a body 762 and a leg 764 extending from the body 762. The stents 776 can include a leading (e.g., proximal-most) stent 776a at and/or defining a leading end portion 761 of the aortic repair device 750 (e.g., of the body 762). The leading stent 776a can have a periodic shape comprising a plurality of leading apices 777 and a plurality of trailing apices 779.

The aortic repair device 750 is in an expanded position in FIG. 7A before, for example, being compressed into the outer catheter 302 (FIG. 3A) and/or after being deployed from the outer catheter 302. The inner catheter 316 of the inner catheter assembly 310 and the release wire 341 can extend through a lumen of the aortic repair device 750. In the illustrated embodiment, the leading tip capture mechanism 342 is a suture loop 745 (“loop 745”) made of fiber(s), cable(s), and/or the like that is threaded through the leading end portion 761 of the aortic repair device 750. More specifically, the graft material 778 can include a plurality of holes or apertures 790 formed therein at and/or proximate to the leading end portion 761, and the loop 745 can be threaded through the apertures 790 in, for example, an over-under pattern. In some embodiments, the apertures 790 are formed adjacent to and/or proximate to corresponding ones of the leading apices 777 of the leading stent 726a. In the illustrated embodiment, the apertures 790 are formed adjacent to alternating ones of the leading apices 777. In other embodiments, some or all of the apertures 790 can be formed along other portions of the leading end portion 761 of the aortic repair device 750 (e.g., adjacent to each of the leading apices 777, adjacent to different ones of the leading apices 777, adjacent to some or all of the trailing apices 779, and/or the like). Accordingly, the loop 745 can extend through some or all of the leading apices 777. In other embodiments, the leading tip capture mechanism 342 can include multiple loops to facilitate a staged release of the leading end portion 761 from the inner catheter assembly 310, as described in detail below.

After threading the loop 745 through the apertures 790, the release wire 341 can be inserted through the loop 745 to releasably secure the loop 745 to the leading end portion 761 of the aortic repair device 750. For example, FIG. 7B is a schematic end-on view of the distal portion of the delivery system 300 and the aortic repair device 750 of FIG. 7A in accordance with embodiments of the present technology. Referring to FIGS. 7A and 7B, the release wire 341 extends through the loop 745 and distally past the aortic repair device 750 to secure the loop 745 in position relative to the aortic repair device 750. That is the release wire 341 can inhibit or even prevent the loop 745 from being pulled out of (e.g., unthreaded from) the apertures 790 in the aortic repair device 750.

FIG. 7C is a perspective side view of the distal portion of the delivery system 300 after compressing the aortic repair device 750 within the outer catheter 302 in accordance with embodiments of the present technology. FIG. 7D is a perspective side view of the distal portion of the delivery system 300 after compressing the aortic repair device 750 within the outer catheter 302 and after positioning the tip member 320 proximate to the leading end portion 303b of the outer catheter 302 (e.g., after moving the delivery system 300 to the delivery position shown in FIG. 3E) in accordance embodiments of the present technology. Referring to FIGS. 7C and 7D, the loop 745 can extend from the aortic repair device 750 and can be stabilized at the tip member 320. For example, the loop 745 can extend through an aperture 746 in the tip member 320. In some embodiments, the loop 745 can be pulled through the aperture 746 to take up slack in the loop 745 introduced by compressing the aortic repair device 750 and/or by positioning the tip member 320 proximate the leading end portion 303b of the outer catheter 302, and can thereafter be fixed (e.g., pinned, tied) to the tip member 320.

FIG. 7E is a schematic end-on view of the distal portion of the delivery system 300 and the aortic repair device 750 of FIGS. 7C and 7D in accordance with embodiments of the present technology. Referring to FIG. 7E, the outer catheter 302 compresses the aortic repair device 750 toward/against the inner catheter 316. Referring to FIGS. 7B-7E, the release wire 341 extends from within the outer catheter 302, through the loop 745, and distally past the aortic repair device 750 to secure the loop 745 in position relative to the aortic repair device 750.

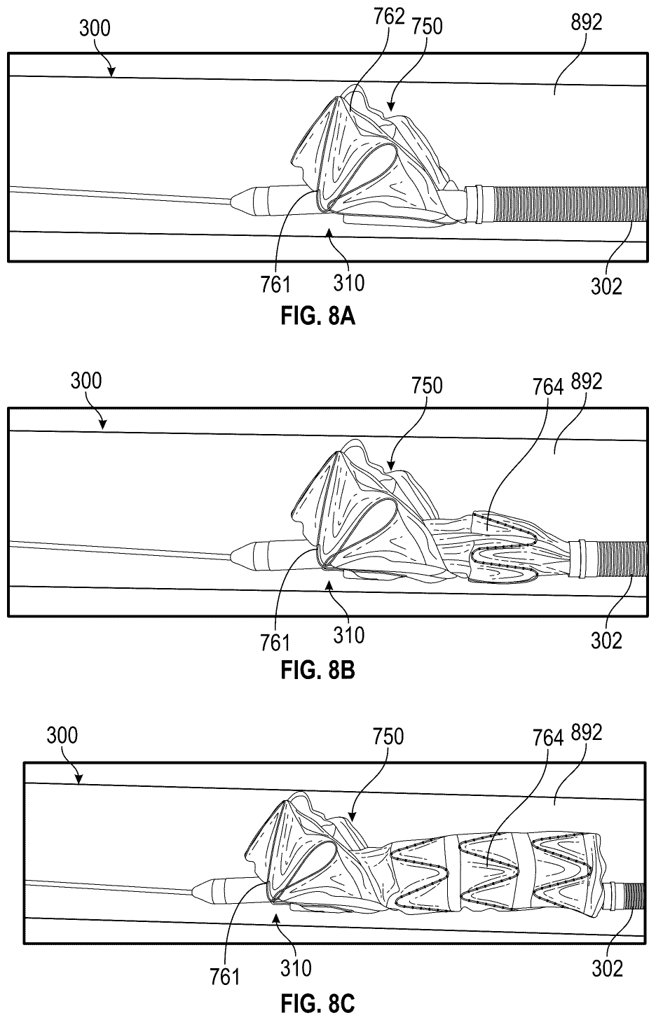

After the aortic repair device 750 is deployed from (e.g., unconstrained by) the outer catheter 302, the loop 745 can hold (e.g., capture) at least a portion of the leading end portion 761 in a constrained (e.g., unexpanded or partially expanded) position, such as the position shown in FIG. 7E. FIGS. 8A-8C, for example, are perspective side views of the delivery system 300 during different stages of deploying the aortic repair device 750 of FIGS. 7A-7E within a lumen of a vessel 892 (e.g., an aorta and/or branch vessel) in accordance with embodiments of the present technology.

Referring to FIG. 8A, the delivery system 300 is shown during a first deployment stage in which the outer catheter 302 has been retracted proximally (e.g., via the actuator 335 shown in FIG. 3A) relative to the inner catheter assembly 310 and the aortic repair device 750 to uncover (e.g., unsheathe) the body 762 of the aortic repair device 750. Referring to FIG. 8B, the delivery system 300 is shown in a second deployment stage in which the outer catheter 302 has been further retracted to uncover a portion of the leg 764 of the aortic repair device 750. Finally, in FIG. 8C, the delivery system 300 is shown in a third deployment stage (e.g., a fully deployed stage) in which the outer catheter 302 has been further retracted to uncover the entirety of the aortic repair device 750. Referring to FIGS. 7A-8C together, the loop 745 holds the leading end portion 761 of the aortic repair device 750 in the constrained position after deployment from the outer catheter 302. That is, the loop 745 can cinch the leading end portion 761 against/to the inner catheter assembly 310. After deploying the aortic repair device 750 from the outer catheter 302, the release wire 341 can be pulled proximally through the loop 745 to release tension on the loop 745 and allow the leading end portion 761 of the aortic repair device 750 to expand. The expansion of the aortic repair device 750 can then drive the loop 745 out of the apertures 790 to decouple/release the leading end portion 761 from the leading tip capture mechanism 342 and the inner catheter assembly 310.

In some aspects of the present technology, the loop 745 exerts a proximal tension force against the leading end portion 761 of the aortic repair device 750 (e.g., as the loop 745 fixes the leading end portion 761 of the aortic repair device 750 to the tip member 320 of the delivery system 300) during deployment that can act to inhibit or even prevent unwanted migration of the aortic repair device 750. For example, when the aortic repair device 750 is deployed within an aorta, blood flowing through the aorta can have a high flow rate and/or high pressure that tends to push the aortic repair device 750 distally through the aorta. Such blood flow can also cause windsocking (e.g., uncontrolled movement) of the aortic repair device 750 as the blood flows against the graft material 778 of the repair device before the lumen of the aortic repair device 750 fully expands against the inner wall of the aorta. Accordingly, the leading tip capture mechanism 342 can help inhibit such unwanted movement and retain the leading end portion 761 of the aortic repair device 750 at a target implantation location—for example, a target implantation location adjacent a diseased portion of the aorta (e.g., a dissection and/or aneurysm). The loop 745 can similarly inhibit the aortic repair device 750 from moving proximally within the outer catheter 302 during manipulation of the outer catheter 302, as the loop 745 fixes the leading end portion 761 of the aortic repair device 750 to the tip member 320 of the delivery system 300.

Referring again to FIG. 7A, the apertures 790 are formed adjacent to alternating ones of the leading apices 777 of the leading stent 726a such that loop 745 only directly cinches the alternating leading apices 777. Accordingly, in some embodiments the other alternating ones of the leading apices 777 without any of the apertures 790 formed nearby can partially expand after deployment. In some aspects of the present technology, such partial expansion can help the aortic repair device 750 better approximate a final deployed position of the aortic repair device 750 after the leading end portion 761 is deployed from the outer catheter 302, while still reducing migration and windsocking of the aortic repair device 750 from blood flow. In other embodiments, the apertures 790 are formed adjacent to each of the leading apices 777, and the loop 745 is inserted through each of the apertures 790, to maximize cinching of the leading end portion 761 against the inner catheter assembly 310 and to correspondingly minimize windsocking and unwanted migration upon deployment.

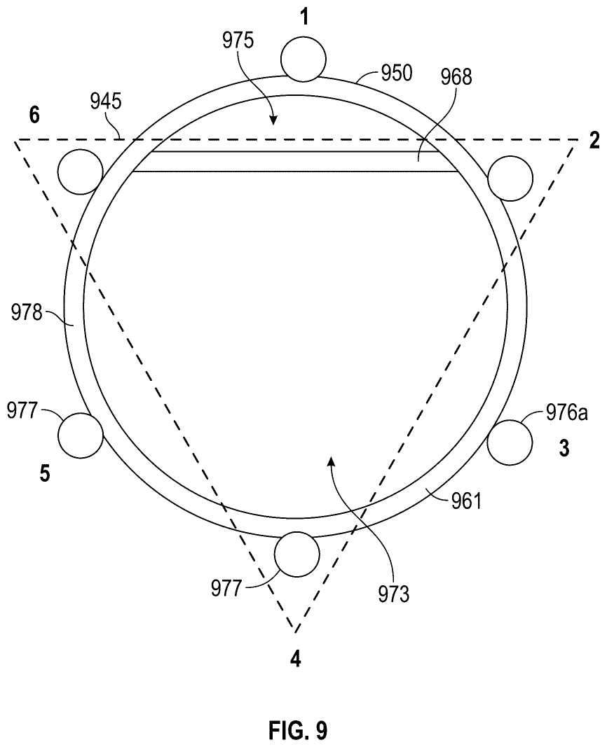

In the illustrated embodiment, the leading stent 726a has ten of the leading and trailing apices 777, 779 such that the loop 745 extends in an over/under pattern through five of the apertures 790. In other embodiments, the leading stent 726a can have more or fewer of the apices. For example, FIG. 9 is a schematic end-on view of a leading end portion 961 of an aortic repair device 950 secured to a suture loop 945 (“loop 945”) of a leading tip capture mechanism of a delivery system in accordance with embodiments of the present technology. The aortic repair device 950 comprises a leading stent 976a secured to a graft material 978 and having six leading apices 977 (individually labeled “1”-“6”), and a septum 968 dividing the aortic repair device 950 into a primary lumen 973 and a secondary lumen 975. The loop 945 is shown schematically in FIG. 9 as to which of the apices 977 the suture extends around or through to secure the suture thereto and allow for cinching of the leading end portion 961 of the aortic repair device 950. In the illustrated embodiment, the loop 945 extends around and secures (e.g., captures) alternating ones of the leading apices 977—such as the 2nd, 4th, and 6th ones of the leading apices 977. More specifically, the loop 945 extends from within the primary lumen 973 and/or the secondary lumen 975 around the alternating ones of the leading apices 977. In some embodiments, the loop 945 can extend out of a first aperture in the graft material 978 proximate to a corresponding one of the leading apices 977 and back through a second aperture in the graft material 978 proximate the corresponding one of the leading apices 977 such that the loop 945 is primarily located within the primary and secondary lumens 973, 975 within the aortic repair device 950. In some aspects of the present technology, this can allow the 1st, 3rd, and 5th one of the leading apices 977 to expand when the aortic repair device 950 is deployed from an outer catheter to approximate a final deployed position of the aortic repair device 950 while still providing some reduction in migration and windsocking of the aortic repair device 950.

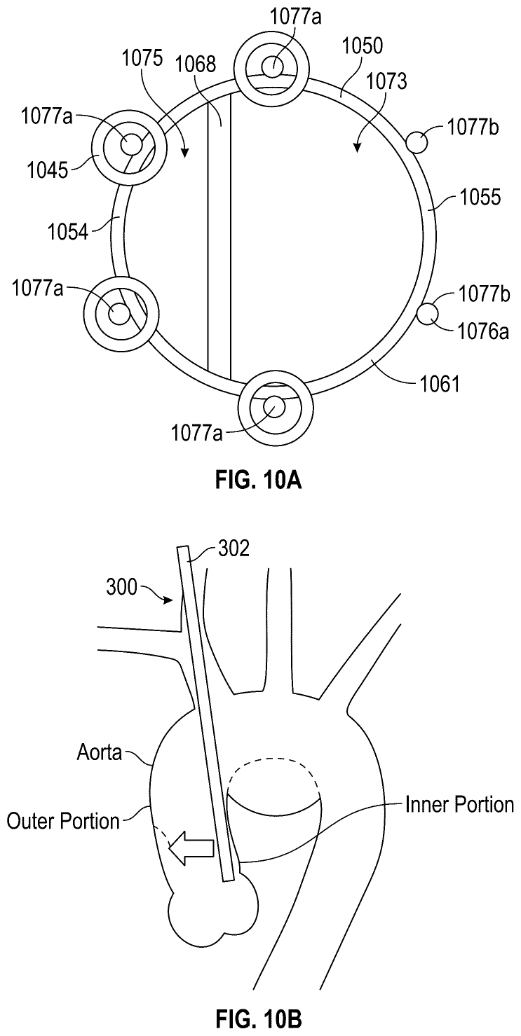

FIG. 10A is a schematic end-on view of a leading end portion 1061 of an aortic repair device 1050 secured to a suture loop 1045 of a leading tip capture mechanism of a delivery system in accordance with embodiments of the present technology. The aortic repair device 1050 comprises a leading stent 1076a having leading apices 1077 (individually labeled as first leading apices 1077a and second leading apices 1077b), and a septum 1068 dividing the aortic repair device 1050 into a primary lumen 1073 and a secondary lumen 1075. The loop 1045 is shown schematically in FIG. 10A as to which of the apices 1077 it extends around or through to secure the suture thereto and allow for cinching of the leading end portion 1061 of the aortic repair device 1050. In the illustrated embodiment, the loop 1045 extends around and secures (e.g., captures) the first leading apices 1077a. In some embodiments, (i) the first leading apices 1077a are positioned adjacent to a first side portion 1054 of the aortic repair device 1050 that is configured to conform to an outer portion (e.g., an outer curved portion) of an interior wall of an aorta having a greater curvature (e.g., greater radius of curvature, longer length), and (ii) the second leading apices 1077b are positioned adjacent to a second side portion 1055 of the aortic repair device 1050 opposite to the first side portion 1054 that is configured to conform to an inner portion (e.g., an inner curved portion) of the interior wall of the aorta having a lesser curvature (e.g., a smaller radius of curvature, shorter length). Accordingly, when the aortic repair device 1050 is deployed from within an outer catheter of a delivery system, the second leading apices 1077b are free to at least partially expand and can help push the aortic repair device 1050 away from the inner portion of the aorta toward the outer portion to help center the aortic repair device 1050 within the aorta.

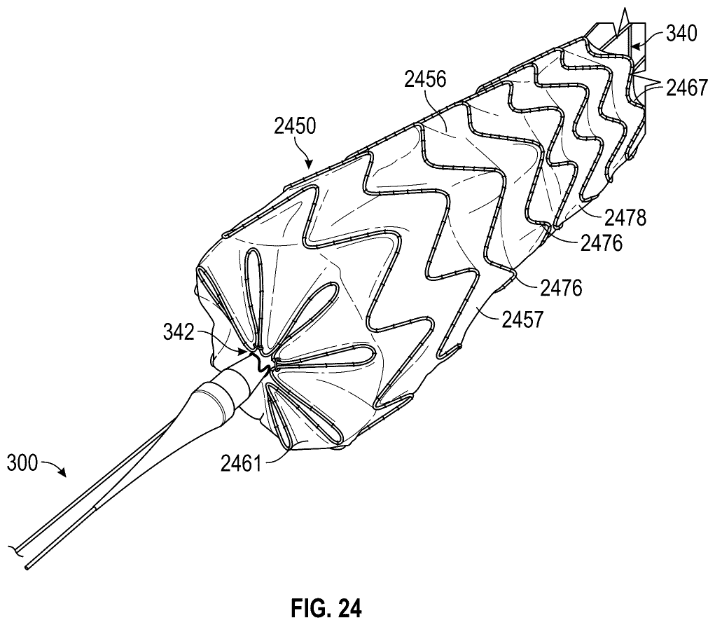

FIG. 10B, for example, is a side view of the delivery system 300 of FIGS. 3A-3G positioned at least partially within an aorta and configured to deploy the aortic repair device 1050 of FIG. 10A within the aorta in accordance with embodiments of the present technology. Referring to FIGS. 10A and 10B, deployment of the aortic repair device 1050 from within the outer catheter 302 can push the aortic repair device 1050 and/or the outer catheter 302 (and the inner catheter assembly 316 shown in FIGS. 3A-3G) away from an inner portion of the aorta toward an outer portion of the aorta in the direction of arrow A. As described in greater detail below with reference to FIGS. 24-34E, in some aspects of the present technology such centering of the delivery system 300 can improve the subsequent deployed position (e.g., squareness) of the aortic repair device 1050 within the aorta.

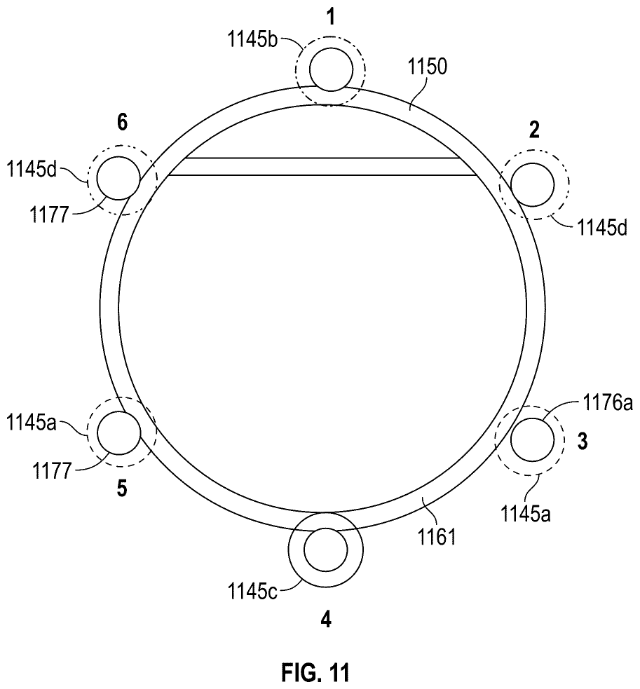

In some embodiments, a delivery system in accordance with embodiments of the present technology can include multiple loops and multiple release wires to facilitate a staged release of the leading end portion of an aortic repair device. FIG. 11, for example, is a schematic end-on view of a leading end portion 1161 of an aortic repair device 1150 secured to multiple loops 1145 (including individually identified first through fourth loops 1145a-d, respectively) of a leading tip capture mechanism of a delivery system in accordance with embodiments of the present technology. The aortic repair device 1150 comprises a leading stent 1176a having six leading apices 1177 (individually labeled “1”-“6”). The loops 1145 are shown schematically in FIG. 11 as to which of the apices 1177 they extend around or through. In the illustrated embodiment, the first loop 1145a extends around and secures the 3rd and 5th ones of the leading apices 1177, the second loop 1145b extends around and secures the 1st one of the leading apices 1177, the third loop 1145c extends around and secures the 4th one of the leading apices 1177, and the fourth loop 1145d extends around and secures the 2nd and 6th ones of the leading apices 1177. Each of the loops 1145 can be releasably secured to a separate release wire of the delivery system that can be individually withdrawn proximally to release the corresponding one of the loops 1145 and the corresponding one(s) of the leading apices 1177. Accordingly, by withdrawing the release wires individually, the leading end portion 1161 of the aortic repair device 1150 can be released from the delivery system in stages-such as in (i) a first stage corresponding to release of the first loop 1145a and the 3rd and 5th ones of the leading apices 1177, (ii) a second stage corresponding to release of the second loop 1145b and the 1st one of the leading apices, (iii) a third stage corresponding to release of the third loop 1145c and the 4th one of the leading apices 1177, and (iv) a fourth stage corresponding to release of the fourth loop 1145d and the 2nd and 6th ones of the leading apices 1177. In some embodiments, the delivery system can include more or fewer of the loops 1145 and corresponding release wires to facilitate more or fewer release stages, and/or multiple ones of the release wires can be actuated together.

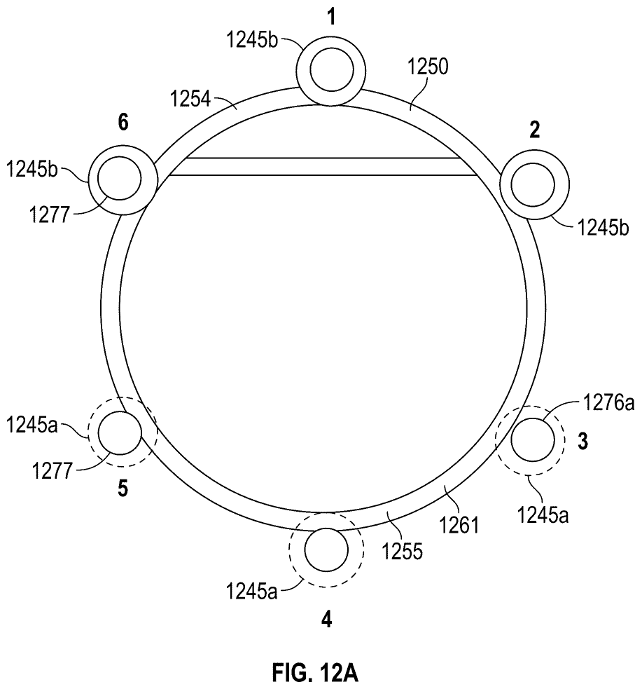

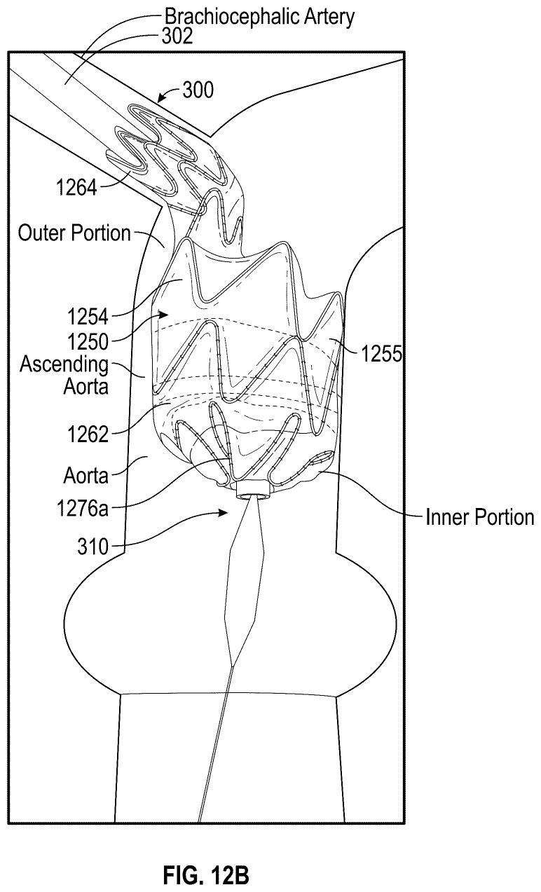

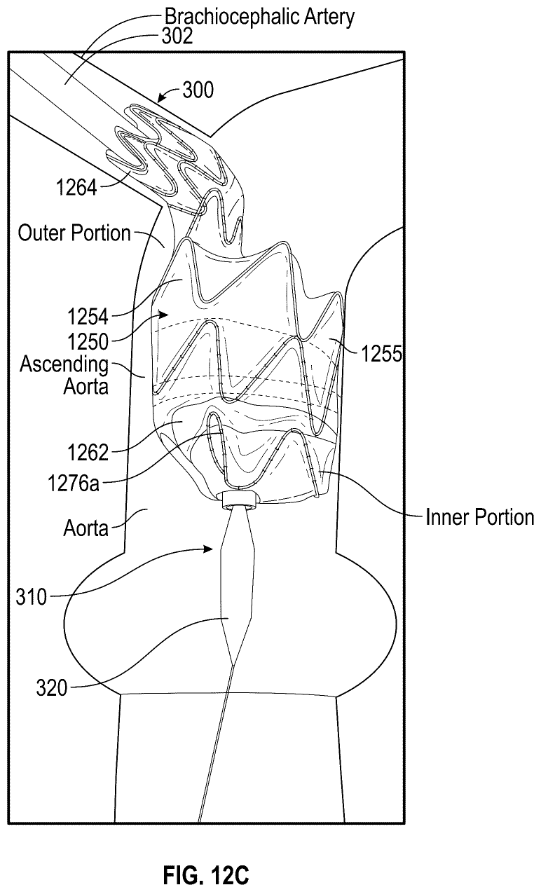

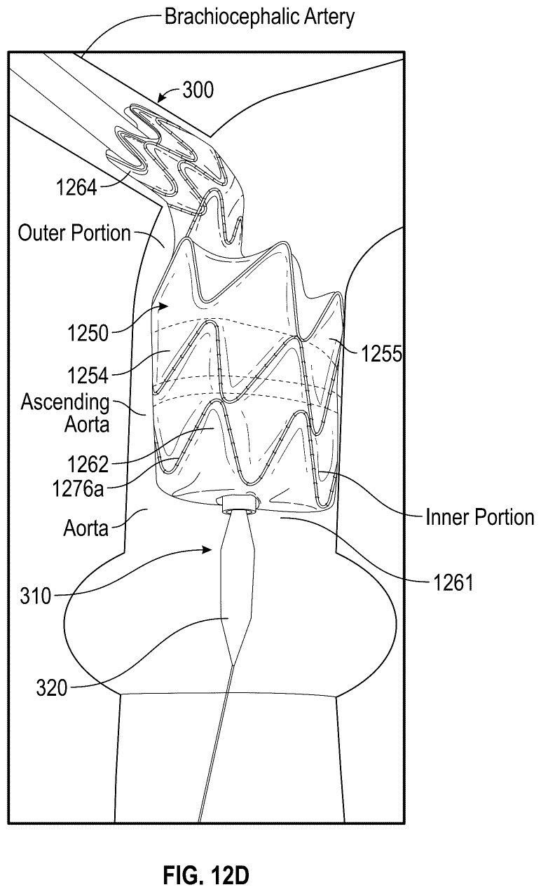

FIG. 12A is a schematic end-on view of a leading end portion 1261 of an aortic repair device 1250 secured to multiple loops 1245 (including an individually identified first loop 1245a and a second loop 1245b) of a leading tip capture mechanism of a delivery system in accordance with embodiments of the present technology. The aortic repair device 1250 comprises a leading stent 1276a having six leading apices 1277 (individually labeled “1”-“6”). The loops 1245 are shown schematically in FIG. 12A as to which of the apices 1277 they extend around or through. In the illustrated embodiment, the first loop 1245a extends around and secures the 3rd through 5th ones of the leading apices 1277, and the second loop 1245b extends around and secures the 1st, 2nd, and 6th ones of the leading apices 1277. Each of the loops 1245 can be releasably secured to a separate release wire (e.g., a first release wire and a second release wire) of the delivery system that can be individually withdrawn proximally to release the corresponding one of the loops 1245 and the corresponding one(s) of the leading apices 1277. In some embodiments, the 1st, 2nd, and 6th ones of the leading apices 1277 are positioned adjacent to a first side portion 1254 of the aortic repair device 1250 that is configured to conform to an outer portion of an interior wall of an aorta having a greater curvature, and (ii) the 3rd through 5th ones of the leading apices 1277 are positioned adjacent to a second side portion 1255 of the aortic repair device 1250 opposite to the first side portion 1254 that is configured to conform to an inner portion of the interior wall of the aorta having a lesser curvature.

FIGS. 12B-12D are perspective side views of the delivery system 300 of FIGS. 3A-3G during different stages of releasing the leading end portion 1261 of the aortic repair device 1250 of FIG. 12A within an aorta in accordance with embodiments of the present technology. Referring to FIGS. 12A-12C, the aortic repair device 1250 is shown deployed from the outer catheter 302 in an expanded position with a body 1262 of the aortic repair device 1250 positioned within the ascending aorta and a leg 1264 of the aortic repair device 1250 positioned at least partially within a branch vessel (e.g., the brachiocephalic artery). The first side portion 1254 of the aortic repair device 1250 is positioned adjacent the outer portion of the aorta and the second side portion 1255 of the aortic repair device 1250 is positioned adjacent the inner portion of the aorta.

Referring to FIGS. 12A and 12B, in a first release stage shown in FIG. 12B, each of the leading apices 1277 of the leading stent 1276a can be initially cinched by the loops 1245 to inhibit migration of the aortic repair device 1250 through the aorta and/or windsocking of the aortic repair device 1250 within the aorta. That is, the loops 1245 cinch and tether the leading end portion 1261 of the aortic repair device 1250 to/against the inner catheter assembly 310.

Referring to FIGS. 12A and 12C, in a second release stage shown in FIG. 12C, the first release wire coupled to the first loop 1245a can be pulled proximally (e.g., through the outer catheter 302) to allow the 3rd through 5th ones of the leading apices 1277 to at least partially expand. In the illustrated embodiment, the expansion of the 3rd through 5th ones of the leading apices 1277 can help push the aortic repair device 1250 away from the inner portion of the aorta toward the outer portion to better center the aortic repair device 1250 within the aorta and/or to improve the squareness of the aortic repair device 1250 within the aorta. In some embodiments, the expansion of the second side portion 1255 of the aortic repair device 1250 can also drive the inner catheter assembly 310 away from the inner portion of the aorta via the engagement of the second loop 1245b with the tip member 320. In the second stage, the 1st, 2nd, and 6th ones of the leading apices 1277 remain cinched against and/or tethered to the inner catheter assembly 310 to inhibit windsocking and/or migration of the aortic repair device 1250.

Referring to FIGS. 12A and 12D, in a third release stage shown in FIG. 12D, the second release wire coupled to the second loop 1245b can be pulled proximally (e.g., through the outer catheter 302) to allow the 1st, 2nd, and 6th ones of the leading apices 1277 to expand and to fully release the leading end portion 1261 of the aortic repair device 1250 from the inner catheter assembly 310. In the third stage, the leading end portion 1261 is fully expanded at a target location within the aorta (e.g., proximate a dissection and/or aneurysm).

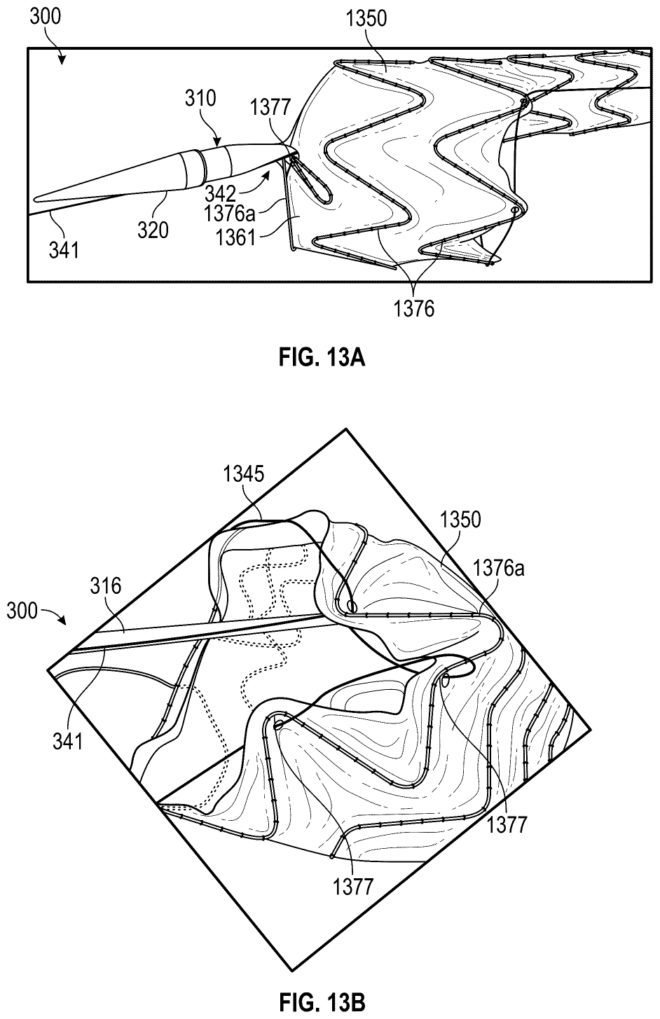

In some embodiments, a delivery system in accordance with embodiments of the present technology can be configured to tightly or loosely secure an end portion (e.g., a leading end portion) of an aortic repair device. For example, FIG. 13A is a perspective view of a distal portion of the delivery system 300 of FIGS. 3A-3G secured to an aortic repair device 1350 in accordance with embodiments of the present technology. The aortic repair device 1350 is shown deployed and expanded from the outer catheter 302 (FIG. 3A) and includes a plurality of stents 1376 including a leading stent 1376a at a leading end portion 1361 of the aortic repair device 1350. The leading stent 1376a has a plurality of leading apices 1377 releasably secured to the tip member 320 via the leading tip capture mechanism 342 (e.g., a loop 1345 shown in FIG. 13B and obscured in FIG. 13A) and the release wire 341. In the illustrated embodiment, the leading apices 1377 are tightly cinched against the inner catheter assembly 310 (e.g., the inner catheter 316 shown in FIGS. 3A, 3B, and 13B). In some aspects of the present technology, tightly cinching the leading end portion 1361 of the aortic repair device 1350 can accurately link the position of the inner catheter assembly 310 with the leading apices 1377 captured by the leading tip capture mechanism 1342.

FIG. 13B is a perspective view of a distal portion of the delivery system 300 of FIGS. 3A-3G secured to the aortic repair device 1350 of FIG. 13A in accordance with additional embodiments of the present technology. In the illustrated embodiment, the loop 1345 is loosely secured to the leading apices 1377 at the leading end portion 1361 of the aortic repair device 1350 such that the leading end portion 1361 is loosely cinched against the inner catheter 1316. In some aspects of the present technology, loosely cinching the leading end portion 1361 of the aortic repair device 1350 can allow the leading end portion 1361 to partially expand after deployment from the outer catheter 302 (FIG. 3A) to approximate a target implantation position for the aortic repair device 1350 within an aorta and/or to slide (e.g., distally) relative to the inner catheter 316 to improve the squareness of the aortic repair device 1350 within the aorta after release from the delivery system 300. More specifically, the windsocking of the leading end portion 1361 can preferentially compress a length of the aortic repair device 1350 along, for example, the inner curvature of the aorta, as the windsocking acts like a sail and pushes the stents 1376 along the inner curvature to move distally, creating a shortening of the length on the inner curve of the aortic repair device 1350, thereby matching the shorter inner curve length of the anatomy better.

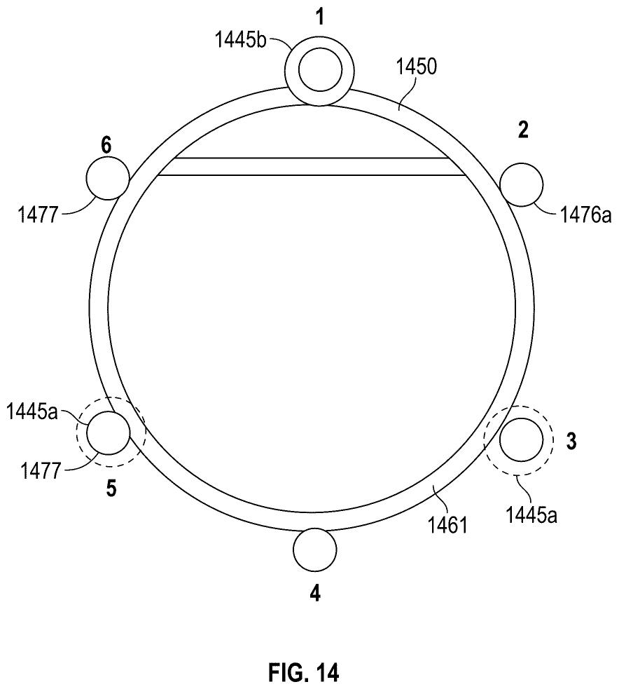

In some embodiments, a delivery system in accordance with embodiments of the present technology can include multiple loops having different tensions to selectively loosely or tightly cinch different apices of an aortic repair device. For example, FIG. 14 is a schematic end-on view of a leading end portion 1461 of an aortic repair device 1450 secured to multiple loops 1445 (including an individually identified first loop 1445a and a second loop 1445b) of a leading tip capture mechanism of a delivery system in accordance with embodiments of the present technology. The aortic repair device 1450 comprises a leading stent 1476a having six leading apices 1477 (individually labeled “1”-“6”). The loops 1445 are shown schematically in FIG. 14 as to which of the apices 1477 they extend around or through. In the illustrated embodiment, the first loop 1445a extends around and secures the 3rd and 5th ones of the leading apices 1477, and the second loop 1445b extends around and secures the 1st one of the leading apices 1477. Each of the loops 1445 can be releasably secured to a separate release wire (e.g., a first release wire and a second release wire) of the delivery system that can be individually withdrawn proximally to release the corresponding one of the loops 1445 and the corresponding one(s) of the leading apices 1477. In some embodiments, the first loop 1445a has a lesser tension than the second loop 1445b such that, for example, the 3rd and 5th ones of the leading apices 1477 are loosely secured to delivery system and the 1st one of the leading apices 1477 is tightly secured to the delivery system. The first release wire can be pulled during a first release stage to release the 3rd and 5th ones of the leading apices 1477, and the second release wire can be pulled during a first release stage to release the 1st one of the leading apices 1477. In some embodiments, the 3rd and 5th ones of the leading apices 1477 are configured to be positioned adjacent an inner portion of an aorta such that their release during the first stage centers the aortic repair device 1450 within the aorta, as described in detail above. In some aspects of the present technology, the lesser tension in the first loop 1445a can allow the corresponding 3rd and 5th ones of the leading apices 1477 to partially expand such that there is some windsocking of the leading end portion 1461 of the aortic repair device 1450 to, for example, permit some tilting of the aortic repair device 1450 to improve squareness of the aortic repair device 1450 within the aorta.

FIG. 15 is an isometric view of a distal portion of a delivery system 1500 including a leading tip capture mechanism 1542 in accordance with embodiments of the present technology. The delivery system 1500 can include some features that are at least generally similar in structure and function, or identical in structure and function, to the corresponding features of the delivery system 300, the delivery system 500, and/or the delivery system 600 described in detail above with reference to FIGS. 3A-3G and 5A-6E, and can operate in a generally similar or identical manner to the delivery system 300 and/or the delivery system 500. For example, the delivery system 1500 includes an inner catheter assembly 1510 configured to be extend through an outer catheter (not shown) and having an inner shaft 1516 coupled to a tip member 1520.

In the illustrated embodiment, the leading tip capture mechanism 1542 is integrated with the tip member 1520 and comprises a body 1549 having a first (e.g., trailing) portion 1543, a second portion (e.g., leading) portion 1544, and a third (e.g., middle) portion 1545. The third portion 1545 can have a smaller diameter than the first and second portions 1543, 1544 such that the third portion 1545 defines a recess 1546 in the body 1549. The body 1549 and the tip member 1520 collectively define a plurality of lumens 1547 extending axially therethrough. The lumens 1547 are configured to receive an individual one of a plurality of release wires 1541 (only one of the release wires 1541 is shown in FIG. 15) therethrough. The third portion 1545 can have an outer surface defining a plurality of circumferential stent receiving portions 1548 (including an individually identified first stent receiving portion 1548a) positioned radially inward (e.g., below) corresponding ones of the lumens 1547. In some embodiments, the third portion 1545 has a polygonal (e.g., hexagonal) shape such that the stent receiving portions 1548 are generally planar.

The leading tip capture mechanism 1542 can be releasably secured to a leading end portion of an aortic repair device by positioning the leading apices of a leading stent of the aortic repair device in the recess 1546 adjacent corresponding ones of the stent receiving portions 1548 and advancing the release wires 1541 through the lumens 1547 over the leading apices to pin the apices to the leading tip capture mechanism 1542 (e.g., via mechanical interference). In the illustrated embodiment, for example, a leading apex 1577 of a leading stent 1576a of an aortic repair device is positioned within the recess 1546 adjacent the first stent receiving portion 1548a. The release wire 1541 is inserted through the corresponding one of the lumens 1547 aligned with the first stent receiving portion 1548a over the leading apex 1577 to secure (e.g., pin) the leading apex 1577 to the leading tip capture mechanism 1542. When the leading apices of the aortic repair device are pinned to body 1549 beneath the release wires 1541, the leading tip capture mechanism 1542 provides tensile strength that inhibits movement of the aortic repair device away from the leading tip capture mechanism 1542. The release wires 1541 can be separately or collectively (e.g., in one or more stages) pulled proximally through the lumens 1547 over the corresponding leading apices to release the leading apices from the leading tip capture mechanism 1542 to release the leading end portion of the aortic repair device.

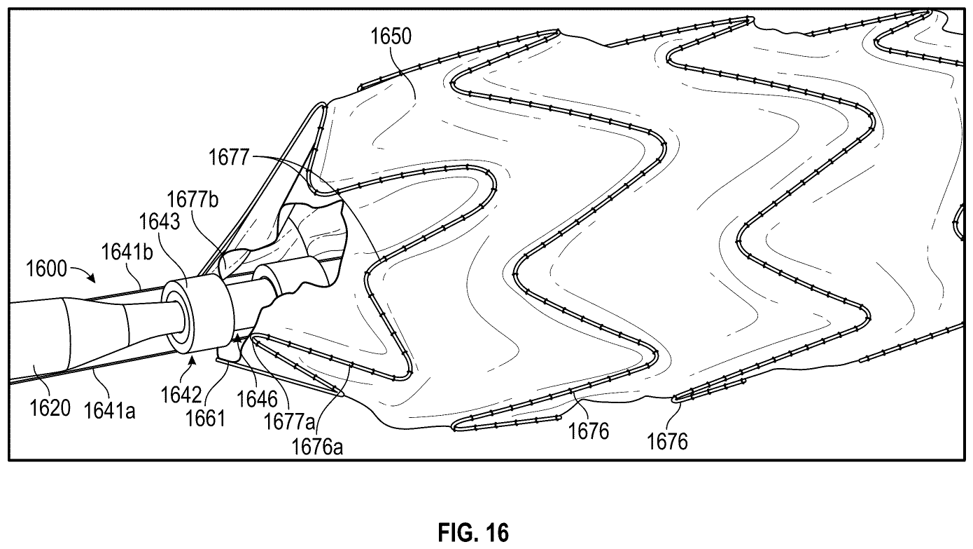

FIG. 16 is a perspective view of a distal portion of a delivery system 1600 including a leading tip capture mechanism 1642 releasably secured to an aortic repair device 1650 in accordance with embodiments of the present technology. In the illustrated embodiment, the aortic repair device 1650 is shown deployed and expanded from an outer catheter (not shown) of the delivery system 1600 and includes a plurality of stents 1676 including a leading stent 1676a at a leading end portion 1661 of the aortic repair device 1650 and having a plurality of leading apices 1677 (including an individually identified first leading apex 1677a and a second leading apex 1677b).

The leading tip capture mechanism 1642 can include some features that are at least generally similar in structure and function, or identical in structure and function, to the corresponding features of the leading tip capture mechanism 1542 described in detail above with reference to FIG. 15 and can operate in a generally similar or identical manner to the leading tip capture mechanism 1542. For example, the leading tip capture mechanism 1642 can include a body 1649 defining a recess 1646 configured to releasably receive one or more of the leading apices 1677 of the aortic repair device 1650. In the illustrated embodiment, the body 1649 has a barbell-like shape defining the recess 1646. The body 1649 and a tip member 1620 of the delivery system 1600 can define one or more lumens for receiving corresponding release wires 1641 (including an individually identified first release wire 1641a and a second release wire 1641b) therethrough. The body 1649 can be coupled to or integrally formed with the tip member 1620. The release wires 1641 can extend over corresponding ones of the leading apices 1677 within the recess 1646 to secure the aortic repair device 1650 thereto and can be removed from the corresponding ones of the lumens to release and deploy the leading end portion 1661 of the aortic repair device 1650.

In the illustrated embodiment, only the first and second leading apices 1677a-b are secured to the leading tip capture mechanism 1642 via the first and second release wires 1641a-b, respectively. In some embodiments, multiple ones of the apices 1677 can overlaid over one another and secured to the same one of the release wires 1641. In other embodiments, more or fewer of the leading apices 1677 can be secured to the leading tip capture mechanism 1642 via corresponding ones of the release wires 1641. The first and second release wires 1641a-b can be pulled proximally together to release the first and second leading apices 1677a-b together, or can be separately pulled proximally separately to release the first and second leading apices 1677a-b in separate stages.

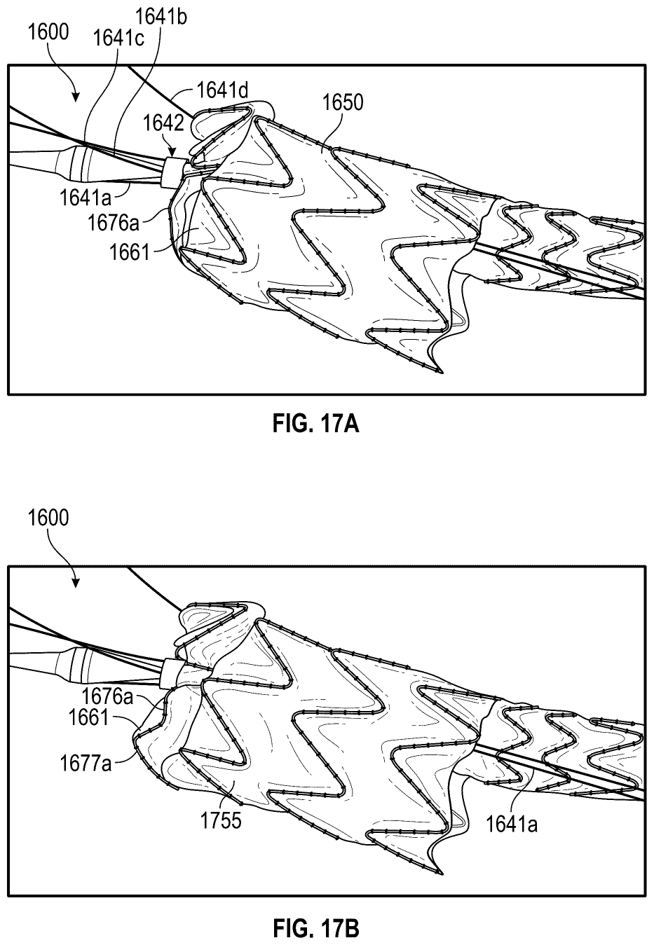

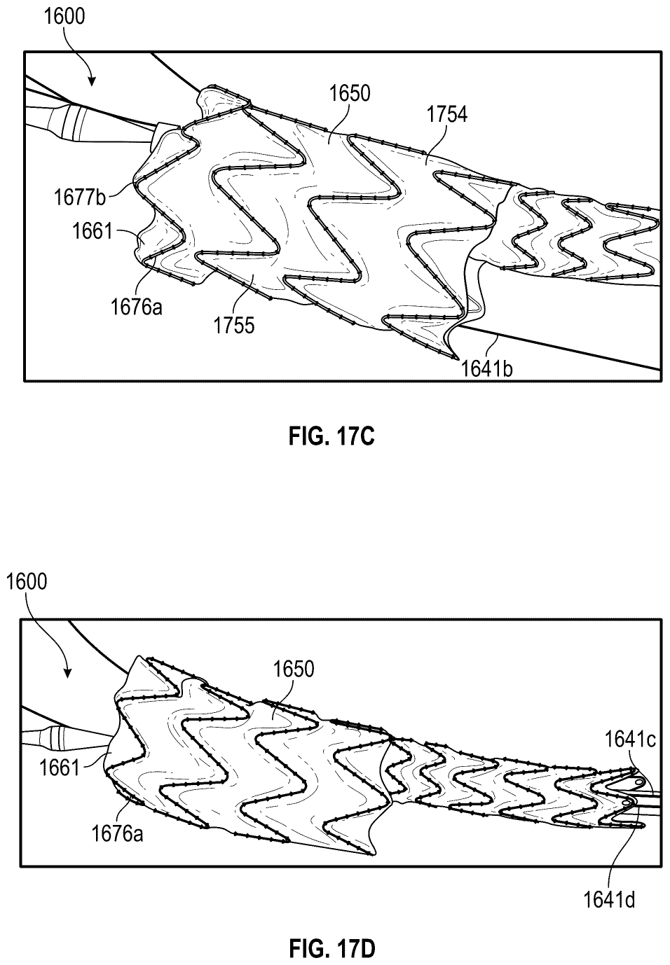

FIGS. 17A-17D are perspective views of a distal portion of the delivery system 1600 and the aortic repair device 1650 of FIG. 16 during different stages of releasing the leading stent 1676a at the leading end portion 1661 of the aortic repair device 1650 in accordance with embodiments of the present technology. Referring to FIG. 17A, in a first release stage, multiple ones of the leading apices 1677 (FIG. 16) are secured to the leading tip capture mechanism 1642 via corresponding one of the release wires 1641 (including individually identified first through fourth release wires 1641a-d, respectively).

Referring to FIG. 17B, in a second release stage, the first release wire 1641a has been withdrawn proximally to release a first one of the leading apices 1677 (identified as first leading apex 1677a). In some embodiments, the first leading apex 1677a is positioned along a second side portion 1755 of the aortic repair device 1650 that is configured to conform to an inner portion of an interior wall of an aorta having a lesser curvature.

Referring to FIG. 17C, in a third release stage, the second release wire 1641b has been withdrawn proximally to release a second one of the leading apices 1677 (identified as second leading apex 1677b). In some embodiments, the second leading apex 1677b is positioned along the second side portion 1755 of the aortic repair device 1650. Accordingly, in some aspects of the present technology the second and third release stages can allow the second side portion 1755 to expand against the inner portion of the aorta to center the aortic repair device 1650 within the aorta, as described in detail above.

Referring to FIG. 17D, in a fourth release stage, the third and fourth release wires 1641c-d have been withdrawn proximally to release corresponding third and fourth ones of the leading apices 1677 (obscured in FIG. 17D). In some embodiments, the third and fourth ones of the leading apices 1677 are positioned along a first side portion 1754 (FIG. 17C) of the aortic repair device 1650 opposite the second side portion 1755 that is configured to conform to an outer portion of the interior wall of the aorta having a greater curvature. In some embodiments, the third and fourth release wires 1641c-d can be withdrawn together to simultaneously or nearly simultaneously release the third and fourth ones of the leading apices 1677 or can be withdrawn separately to release the third and fourth ones of the leading apices 1677 in separate stages.

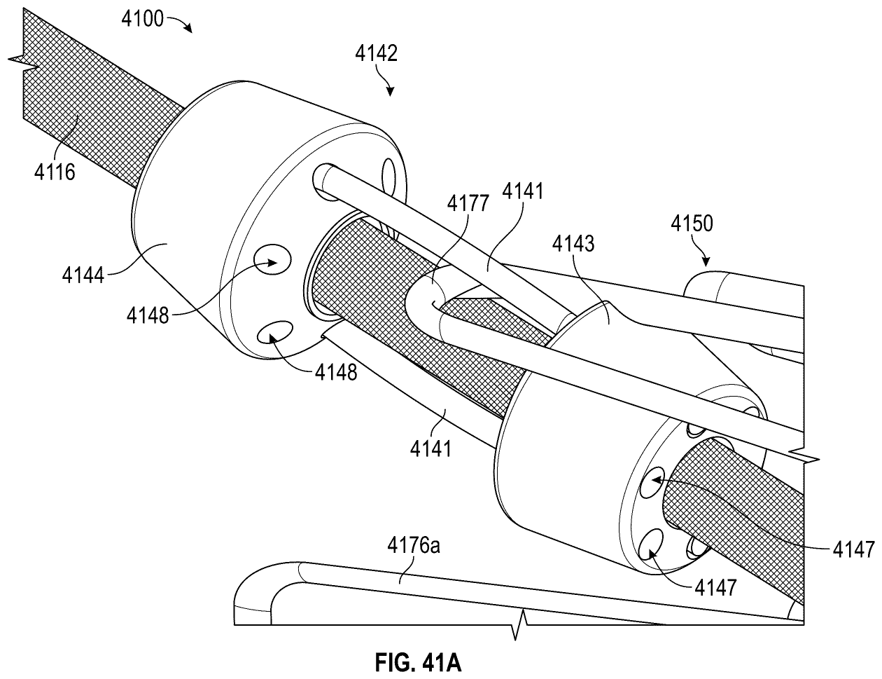

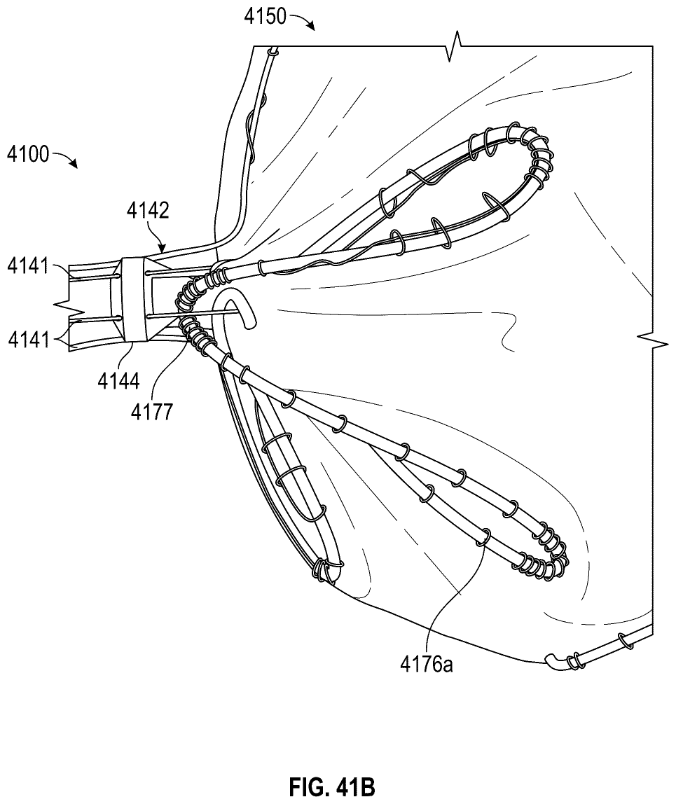

FIG. 41A is an isometric view of a distal portion of a delivery system 4100 including a leading tip capture mechanism 4142 in accordance with embodiments of the present technology. In the illustrated embodiment, the delivery system 4100 includes an inner catheter 4116. The leading tip capture mechanism 4142 can include some features that are at least generally similar in structure and function, or identical in structure and function, to the corresponding features of the leading tip capture mechanism 1542 and/or the leading tip capture mechanism 1642 described in detail above with reference to FIGS. 15-17D, and can operate in a generally similar or identical manner to the leading tip capture mechanism 1542 and/or the leading tip capture mechanism 1642. For example, in the illustrated embodiment the leading tip capture mechanism 4142 includes a first (e.g., trailing) body member 4143 and a second (e.g., leading) body member 4144. The first and second body members 4143, 4144 can be spaced apart from one another along the inner catheter 4116 and can be directly attached thereto (e.g., via welding, adhesives, fasteners). In some embodiments, the second body member 4144 can be incorporated into a tip of the delivery system 4100 (e.g., the tip 320 of FIGS. 3A and 3B). The first body member 4143 defines a plurality of first lumens 4147 extending therethrough (e.g., axially therethrough), and the second body member 4144 define a plurality of second lumens 4148 extending therethrough. The first lumens 4147 can be axially aligned with the second lumens 4148. Pairs of the first and second lumens 4147, 4148 are configured to receive an individual one of a plurality of release wires 4141 (only two of the release wires 4141 are shown in FIG. 41A) therethrough.

The leading tip capture mechanism 4142 can be releasably secured to a leading end portion of an aortic repair device 4150 by positioning the leading apices of a leading stent of the aortic repair device in the space between the first and second body members 4143, 4144 adjacent the inner catheter 4116 and advancing the release wires 4141 through the first and second lumens 4147, 4148 over the leading apices to pin the apices to the inner catheter 4146 (e.g., via mechanical interference). In the illustrated embodiment, for example, a leading apex 4177 of a leading stent 4176a of an aortic repair device is positioned between the first and second body members 4143, 4144 adjacent the first inner catheter 4116. An upper one of the release wires 4141 is inserted through the corresponding one of the first and second lumens 4147, 4148 over the leading apex 4177 to secure (e.g., pin) the leading apex 4177 to the leading tip capture mechanism 4142. When the leading apices of the aortic repair device are pinned to body inner catheter 4116 beneath the release wires 4141, the leading tip capture mechanism 4142 provides tensile strength that inhibits movement of the aortic repair device away from the leading tip capture mechanism 4142. The release wires 4141 can be separately or collectively (e.g., in one or more stages) pulled proximally through the first and second lumens 4147, 4148 over the corresponding leading apices to release the leading apices from the leading tip capture mechanism 4142 to release the leading end portion of the aortic repair device.

In some aspects of the present technology, the leading tip capture mechanism 4142 has a greater flexibility and lower profile than, for example, the leading tip capture mechanisms 1542 and 1642 described in detail above with reference to FIGS. 15-17D. In particular, the leading tip capture mechanism 4142 does not include any material (e.g., metal) extending between the first and second body members 4143, 4144 such that (i) the leading stent 4176a is pinned directly against the inner catheter 4116, thereby minimizing the profile of the leading tip capture mechanism 4142, and (ii) the flexibility of the inner catheter 4116 is not impeded between the first and second body members 4143, 4144. Moreover, in some embodiments the first and second body members 4143, 4144 are identical to reduce fabrication cost and complexity.

FIG. 41B is a perspective view of a distal portion of the leading tip capture mechanism 4142 of the delivery system 4100 of FIG. 41A releasably secured to the aortic repair device 4150 in accordance with embodiments of the present technology. In the illustrated embodiment, multiple (e.g., six) of the release wires 4141 are secured over corresponding leading apices 4177 of the leading stent 4176a between the first body member 4143 (obscured in FIG. 41B) and the second body member 4144. In some embodiments, the leading tip capture mechanism 4142 can include fewer or more of the release wires 4141 than shown in FIG. 41B and each of the release wires 4141 can be secured to corresponding leading apex 4177.

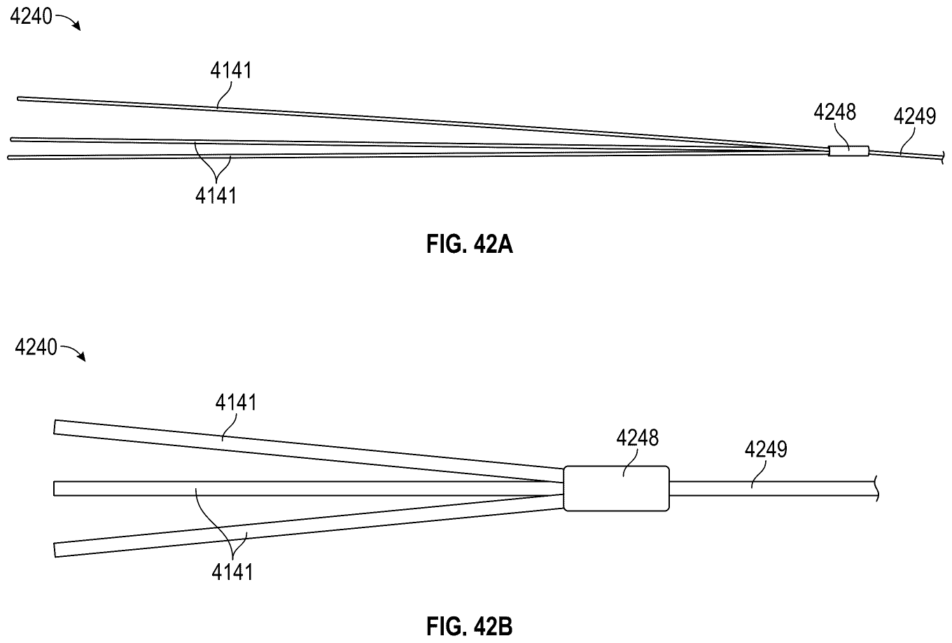

FIG. 42A is a perspective side view of a release wire assembly 4240 that can be used in the delivery system 4100 of FIGS. 41A and 41B—or any of the delivery systems described herein—in accordance with embodiments of the present technology. FIG. 42B is an enlarged side view of a proximal portion of the release wire assembly of FIG. 42A in accordance with embodiments of the present technology. Referring to FIGS. 42A and 42B, multiple ones of the release wires 4141 are secured together at a trailing portion thereof to a hub 4248. The hub 4248 can include a rigid hypotube or other structure (e.g., made of metal, stainless steel) that crimps together the release wires 4141 such that they are constrained to move together. The release wire assembly 4240 can further include a single pull wire 4249 that can be retracted to retract the multiple release wires 4141 together to thereby simultaneously release multiple ones of the leading apices 4177 of the aortic repair device 4150 (FIGS. 41A and 41B). Referring to FIGS. 41A-42B, multiple ones of the release wire assemblies 4240 can be used in the delivery system 4100 having the same or different numbers of coupled release wires 4141 to facilitate the staged deployment of the leading stent 4176a. Similarly, some or all of the release wires 4141 can have differing lengths such that retraction of the single pull wire 4249 (and/or another actuator) operates to retract the release wires 4141 together. Due to the varied lengths of the release wires 4141, the leading stent 4176a still deploys in stages based on the length of the corresponding release wires 4141 (e.g., with leading ones of the apices 4177 coupled to shorter ones of the release wires 4141 releasing before leading ones of the apices 4177 coupled to longer ones of the release wires 4141).

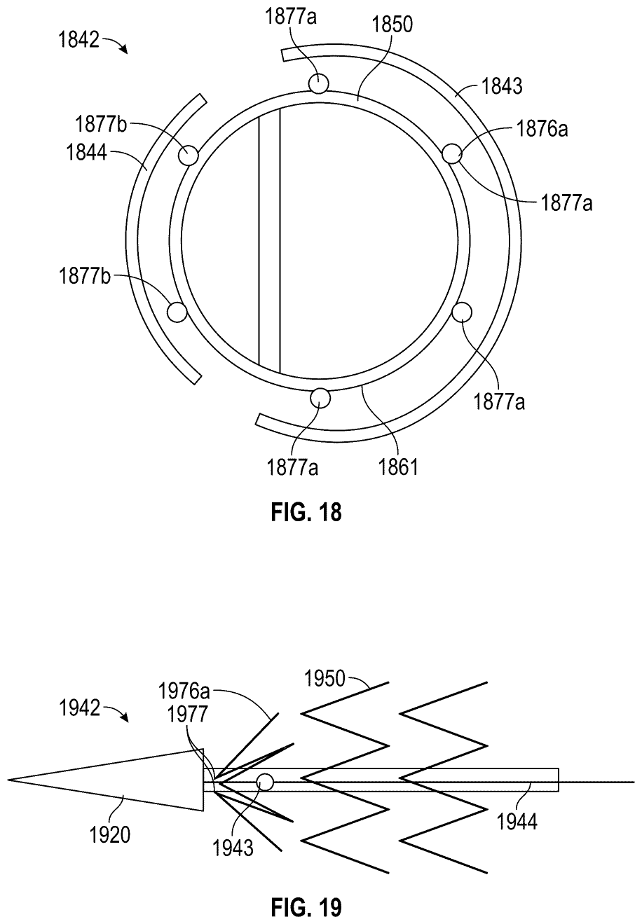

FIG. 18 is an end-on view of a leading end portion 1861 of an aortic repair device 1850 secured to a leading tip capture mechanism 1842 of a delivery system in accordance with embodiments of the present technology. The aortic repair device 1850 can include a leading stent 1876a having six leading apices 1877 (including individually identified first leading apices 1877a and second leading apices 1877b). In the illustrated embodiment, the leading tip capture mechanism 1842 includes a first sleeve 1843 positioned to releasably secure (e.g., capture via mechanical interference) the first leading apices 1877a and a second sleeve 1844 positioned to releasably secure the second leading apices 1877b. The first sleeve 1843 and the second sleeve 1844 are shown schematically in FIG. 18 as to which apices 1877 they cover/secure. The first and second sleeves 1843, 1844 can be actuated (e.g., withdrawn proximally) separately or in stages to release the leading apices 1877.

FIG. 19 is a side view of an aortic repair device 1950 secured to a leading tip capture mechanism 1942 of a delivery system in accordance with embodiments of the present technology. The aortic repair device 1950 can include a leading stent 1976a having leading apices 1977. In the illustrated embodiment, the leading tip capture mechanism 1942 includes a ball 1943 mounted on a wire 1944. The ball 1943 can have a diameter selected to be larger than an area defined by a corresponding one or more of the leading apices 1977 when the aortic repair device 1950 is sheathed and compressed within an outer catheter. Accordingly, the ball 1943 can act as a plug that inhibits the leading apices 1977 from sliding back during unsheathing of the aortic repair device 1950 from the outer catheter. In such embodiments, the wire 1944 can have an axial stiffness selected to inhibit or even prevent the wire 1944 from sliding during unsheathing. In some embodiments, the wire 1944 has a lateral stiffness sufficient to inhibit the leading stent 1976a from self-expanding after unsheathing from the outer catheter. In other embodiments, the delivery system can include a tip member 1920 shaped to at least partially retain the leading apices 1977 and inhibit them from expanding after unsheathing of the aortic repair device. For example, a trailing end portion of the tip member 1920 can be “cupped” or otherwise shaped to retain the leading apices 1977. In such embodiments, the ball 1943 can act as a plug and force the leading apices 1977a against the trailing end portion of the tip member 1920. In any of these embodiments, pulling the wire 1944 proximally can release the leading apices 1977a and allow them to expand.

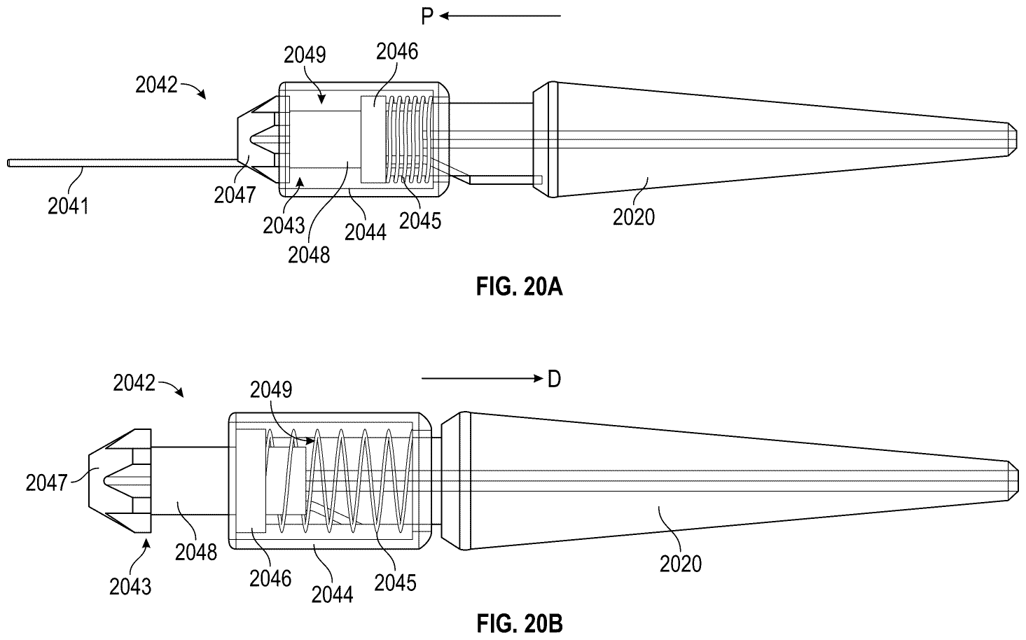

FIGS. 20A and 20B are side views of a leading tip capture mechanism 2042 of a delivery system in accordance with embodiments of the present technology. The leading tip capture mechanism 2042 is in a secured (e.g., first) position in FIG. 20A and in a released (e.g., second) position in FIG. 20B. Referring to FIGS. 20A and 20B, the leading tip capture mechanism 2042 includes a tip member 2020, a capture member 2043 coupled (e.g., fixed, mounted) to the tip member 2020, and a sleeve 2044 (shown as partially transparent in FIGS. 20A and 20B for clarity) positioned at least partially around the capture member 2043 in the secured position (FIG. 20A) and coupled to the capture member 2043 via a biasing member 2045 (e.g., a spring). More specifically, the capture member 2043 can include a mount portion 2046, a head portion 2047, and a rod portion 2048 extending between the mount portion 2046 and the head portion 2047. The sleeve 2044 defines a lumen 2049 extending therethrough, and the biasing member 2045 is positioned within the lumen 2049 and coupled between an end portion of the sleeve 2044 and the mount portion 2046 of the capture member 2043.

Referring to FIG. 20A, in the secured position, the sleeve 2044 can be moved proximally as indicated by arrow P against the biasing force of the biasing member 2045 to, for example, compress and load the biasing member 2045. The leading tip capture mechanism 2042 can further include a release wire 2041 that extends at least partially past the biasing member 2045 and that is configured (e.g., shaped, sized, positioned) to retain the biasing member 2045 in the compressed position. That is, the release wire 2041 can exert a retaining force against the biasing force of the biasing member 2045 to maintain the leading tip capture mechanism 2042 in the secured position. In the secured position, the sleeve 2044 is positioned at least partially over the rod portion 2048 of the capture member 2043. The leading apices of an aortic repair device can be secured within the lumen 2049 of the sleeve 2044 between the rod portion 2048 of the capture member 2043 and the sleeve 2044. The sleeve 2044 can inhibit or even prevent the apices from flexing outwardly and thus secures the apices to the leading tip capture mechanism 2042. In some embodiments, the head portion 2047 can help maintain the apices within the lumen 2049 between the rod portion 2048 and the sleeve 2044 by, for example, inhibiting or even preventing the apices from moving proximally out of the lumen 2049.

To move the leading tip capture mechanism 2042 from the secured position to the release position (FIG. 20A), the release wire 2041 can be withdrawn proximally to release the retaining force against the biasing member 2045. Referring to FIG. 20B, the biasing member 2045 can then drive the sleeve 2044 distally in the direction of arrow D to uncover (e.g., move from within the lumen 2049) at least a portion of the rod portion 2048. The apices of the aortic repair device positioned therein can then flex outwardly and release from the leading tip capture mechanism 2042.

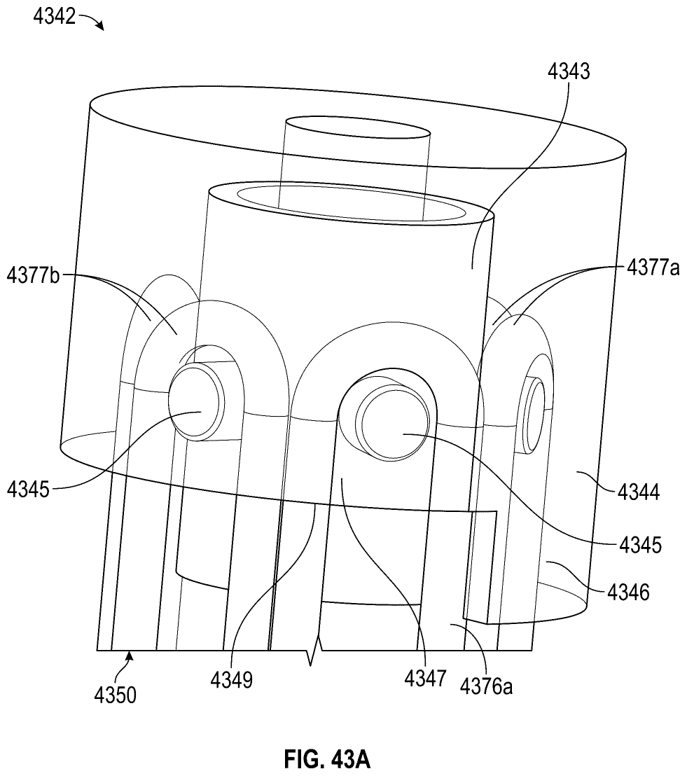

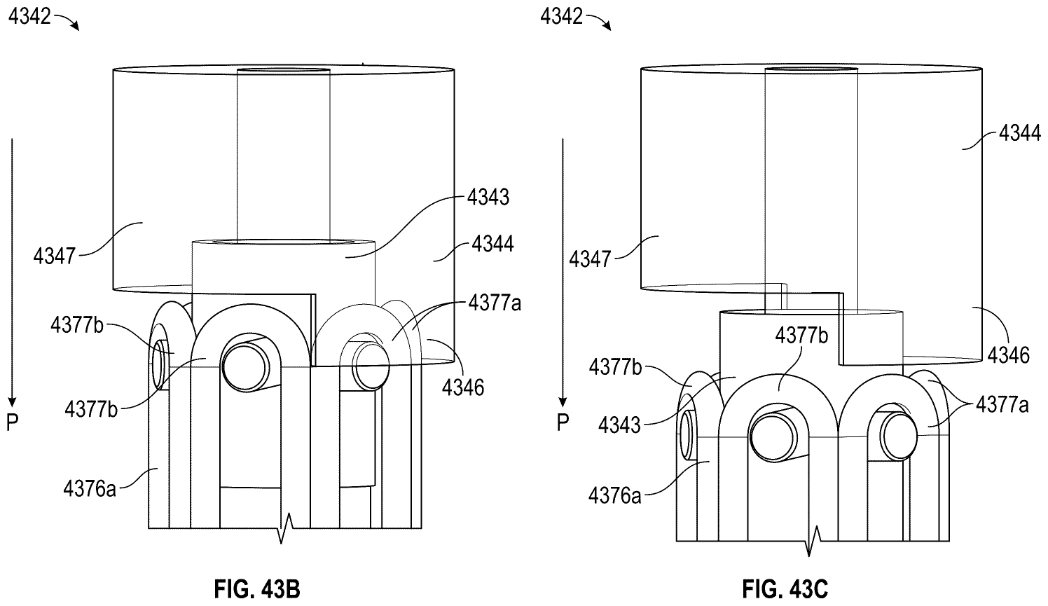

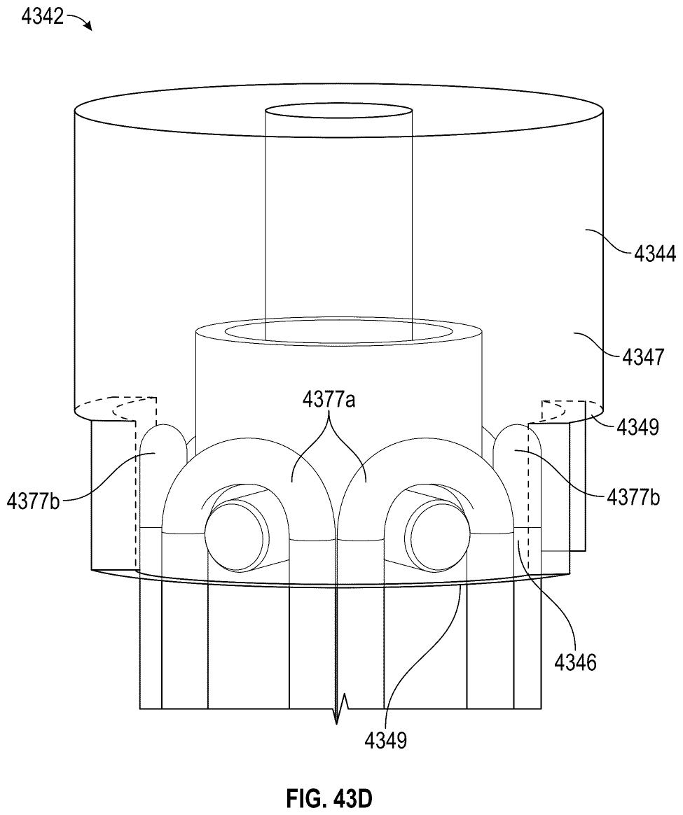

FIG. 43A is an isometric view of a leading stent 4376a of an aortic repair device 4350 secured to a leading tip capture mechanism 4342 of a delivery system in accordance with embodiments of the present technology. The leading tip capture mechanism 4342 can include some features that are at least generally similar in structure and function, or identical in structure and function, to the corresponding features of the leading tip capture mechanism 2042 described in detail above with reference to FIGS. 20A and 20B and can operate in a generally similar or identical manner to the leading tip capture mechanism 2042. For example, the leading tip capture mechanism 4342 includes (i) a capture member 4343 that can be secured to, for example, an inner catheter of the delivery system (e.g., the inner catheter 316 of FIG. 3B) and (ii) a sleeve 4344 (shown as partially transparent in FIG. 43A for clarity; e.g., a cap) positioned at least partially around the capture member 4343.

In the illustrated embodiment, the capture member 4343 includes a plurality of radially outward extending pins 4345. In FIG. 43A, the leading tip capture mechanism 4342 is in a secured (e.g., first) position in which leading apices 4377 (including individually identified first leading apices 4377a and second leading apices 4377b) of the leading stent 4376a are secured around corresponding ones of the pins 4345, and the sleeve 4344 is positioned over the leading apices 4377 to inhibit the leading apices 4377 from extending radially outward. In the secured position, the pins 4345 inhibit the leading apices 4377 from sliding out from under the sleeve 4344. The sleeve 4344 includes a trailing edge 4349 having step that divides the sleeve into a first portion 4346 and a stepped second portion 4347. Accordingly, the first portion 4346 extends farther in the trailing direction than the second portion 4347. The first leading apices 4377a can be positioned proximate to (e.g., covered and constrained) by the first portion 4346 of the sleeve 4344 and the second leading apices 4377b can be positioned proximate to the second portion 4347 of the sleeve 4344. In some embodiments, the first portion 4346 spans circumferentially about adjacent ones (e.g., three) of the first leading apices 4377a, and the second portion 4347 spans circumferentially about adjacent ones (e.g., three) of the second leading apices 4377b.

The leading tip capture mechanism 4342 can be actuated to move (e.g., translate) the capture member 4343 relative to the sleeve 4344 and/or the sleeve 4344 relative to the capture member 4343. For example, the capture mechanism 4342 can be a tube that can be pulled proximally over an inner catheter of the delivery system (e.g., via a mechanism in a handle of the delivery system). Accordingly, the capture mechanism 4342 can be pulled proximally away from the sleeve 4344 by a component of the delivery system to: (i) first uncover the second leading apices 4377b proximate the second portion 4347 to allow the second leading apices 4377b to flex outwardly and release from the leading tip capture mechanism 4342; and (ii) then uncover the first leading apices 4377a proximate the first portion 4346 to allow the first leading apices 4377a to flex outwardly and release from the leading tip capture mechanism 4342. More specifically, FIGS. 43B and 43C are isometric views of the leading tip capture mechanism 4342 in a first release position (e.g., second position) and a second release position (e.g., a third position), respectively, in accordance with embodiments of the present technology. Referring to FIG. 43B, in the first release position, the capture member 4343 has been translated proximally in the direction of arrow P away from the sleeve 4344 by a first distance such that the second leading apices 4377b of the leading stent 4376a proximate the second portion 4347 can flex outwardly and release from the leading tip capture mechanism 4342. In the first release position, the first leading apices 4377a remain covered by the first portion 4346 of the sleeve 4344 and radially constrained thereby. Referring to FIG. 43C, in the second release position, the capture member 4343 has been translated proximally in the direction of arrow P farther away from the sleeve 4344 by a second distance, greater than the first distance, such that the first leading apices 4377a of the leading stent 4376a proximate the first portion 4346 can flex outwardly and release from the leading tip capture mechanism 4342. Accordingly, the leading tip capture mechanism 4342 is configured (e.g., shaped, sized) to facilitate the staged release of the leading apices 4377.

In some embodiments, the sleeve 4344 can have other configurations (e.g., shapes, sizes) to facilitate different staged deployment of the leading apices 4377a. For example, FIG. 43D is an isometric view of the leading tip capture mechanism 4342 in accordance with additional embodiments of the present technology. In the illustrated embodiment, the trailing edge 4349 has a pair of opposing steps such that the first portion 4346 of the sleeve 4344 covers opposing pairs of the first leading apices 4377a, and the second portion 4347 of the sleeve 4344 covers opposing ones of the second leading apices 4377b. Accordingly, the second leading apices 4377b are released first in the first release position, and the first leading apices 4377a are released next in the second release position.

Referring to FIGS. 43A-43D, in other embodiments the sleeve 4344 can have a uniform length/height (e.g., without a stepped or variable height trailing edge 4349), and the pins 4345 can be staggered along the capture member 4343 at different longitudinal positions. The staggered arrangement of the pins 4345 can provide for a staged release of the leading apices 4377 of the leading stent 4376a. In some such embodiments, the leading stent 4376a can be non-uniform in height such that the height of the leading apices 4377 corresponds to the differing positioning of the pins 4345.

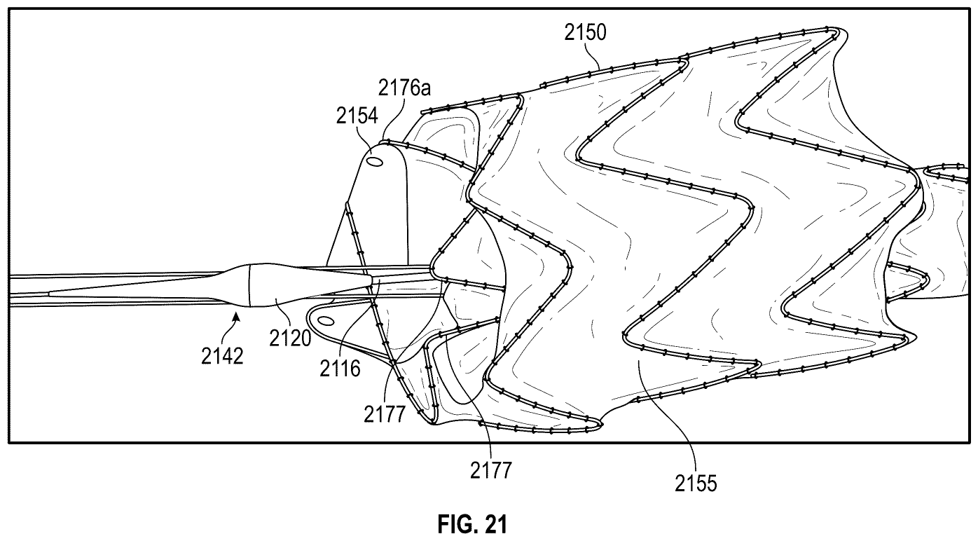

FIG. 21 is a side view of an aortic repair device 2150 secured to a leading tip capture mechanism 2142 of a delivery system in accordance with embodiments of the present technology. The aortic repair device 2150 can include a leading stent 2176a having leading apices 2177. In the illustrated embodiment, the leading stent 2176a is a wedge stent having a longer/taller stent height on one side (e.g., a first side portion 2154 of the aortic repair device 2150) and a shorter stent height on the other side (e.g., a second side portion 2155 of the aortic repair device 2150). On the shorter side of the leading stent 2176a along the second side portion 2155, the tip capture mechanism 2142 can be tied (e.g., via a suture loop) to the leading stent 2176a with additional length to allow the shorter side of the leading stent 2176a to slide along an inner shaft 2116 of the delivery system to compensate for the difference in the height of the leading stent 2176a. The taller side of the leading stent 2176a along the first side portion 2154 can be tied with a second tip capture suture loop of the tip capture mechanism 2142 that cinches the leading apices 2177 of the leading stent 2176a to a tip member 2120 of the delivery system. In other embodiments, the leading stent 2176a can have a constant or substantially constant height but can still be secured via different suture lengths to the tip capture mechanism 2142. In such embodiments, adding additional length to one stage of the tip capture can allow one side of the leading stent 2176 to retract with the unsheathing of the outer catheter of the delivery system to help move the side of the leading stent 2176 to a more favorable deployment position prior to releasing the leading stent 2176a from the tip capture mechanism 2142.

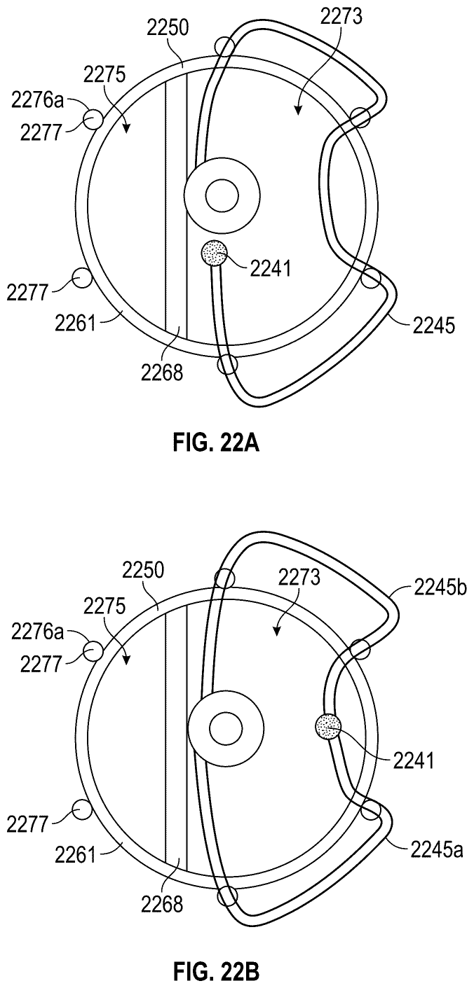

FIGS. 22A and 22B are schematic end-on views of a leading end portion 2261 of an aortic repair device 2250 secured a tip capture mechanism of a delivery system in accordance with embodiments of the present technology. Referring to FIGS. 22A and 22B, the aortic repair device 2250 comprises a leading stent 2276a having leading apices 2277, and a septum 2268 dividing the aortic repair device 2250 into a primary lumen 2273 and a secondary lumen 2275.

Referring to FIG. 22A, the tip capture mechanism includes a suture loop 2245 that extends around and secures (e.g., captures) the leading apices 2277 adjacent the primary lumen 2273. That is, the single loop 2245 is a continuous path to secure four consecutive ones of the leading apices 2277. In other embodiments, the loop 2245 can extend around and secure different ones of the leading apices 2277, and/or more or fewer of the leading apices 2277. A release wire 2241 extends through the loop 2245, and distally past the aortic repair device 2250 to secure the loop 2245 in position relative to the aortic repair device 2250.

Referring to FIG. 22B, the tip capture mechanism includes a first loop 2245a and a second loop 2245b that extend around and secure (e.g., capture) the leading apices 2277 adjacent the primary lumen 2273. The release wire 2241 extends through the first and second loops 2245a-b, and distally past the aortic repair device 2250 to secure the first and second loops 2245a-b in position relative to the aortic repair device 2250. In the illustrated embodiment, the first loop 2245a traverses in a direction counter to the second loop 2245b and is releasably joined to the second loop 2245b with the release wire 2241. Referring to FIGS. 22A and 22B, when the release wire 2241 is pulled proximally, in some aspects of the present technology the shorter symmetric paths of the first and second loops 2245a-b of FIG. 22B can yield a faster and more balanced opening of the leading apices 2277 in comparison to the single loop 2245 of FIG. 22A.

Referring to FIGS. 7A-22B and 41A-43D, although the various tip capture mechanisms are often described in the context of capturing and securing a leading end portion of an aortic repair device, the various tip capture mechanisms can additionally or alternatively be integrated into a delivery system to capture and secure a trailing end portion of an aortic repair device. Accordingly, a trailing tip capture mechanism in accordance with embodiments of the present technology can releasably capture and secure a trailing stent (e.g., trailing apices thereof) of an aortic repair device via any of the devices and methods described above.

In some embodiments, for example, a trailing tip capture mechanism comprises one or more loops releasably secured to a trailing end portion of an aortic repair device via one or more release wires, as described in detail above with reference to FIGS. 7A-14. More specifically, for example, FIG. 23 is a perspective side view of a distal portion of the delivery system 300 of FIGS. 3A-3G secured to an aortic repair device 2350 in accordance with embodiments of the present technology. In the illustrated embodiment, the aortic repair device 2350 is shown deployed and expanded from the outer catheter 302 (FIG. 3A) of the delivery system 300 and includes a plurality of stents 2376 coupled to a graft material 2378 and including a leading stent 2376a at a leading end portion 2361 of the aortic repair device 2350 and a trailing stent 2376b at a trailing end portion 2367 of the aortic repair device 2350. The leading stent 2376a can be secured to the leading tip capture mechanism 342 as described in detail above with reference to FIGS. 7A-22B and 41A-43D.

The trailing stent 2376b can have a periodic shape comprising a plurality of leading apices 2397 and a plurality of trailing apices 2399. In the illustrated embodiment, the trailing tip capture mechanism 340 comprises a loop 2395 of fiber, suture, cable, and/or the like that is threaded through the trailing end portion 2367 of the aortic repair device 2350. The loop 2395 can extend from the aortic repair device 2350 and can be stabilized at the inner catheter assembly 310 or another component of the inner catheter assembly 310. More specifically, the graft material 2378 can include a plurality of holes or apertures formed therein at and/or proximate to the trailing end portion 2367, and the loop 2395 can be threaded through the apertures. In some embodiments, the apertures 2390 are formed adjacent to and/or proximate to corresponding ones of the trailing apices 2399 of the trailing stent 2376b. The apertures can be formed adjacent to alternating ones of the trailing apices 2399, adjacent to each of the trailing apices 2399, along other portions of the trailing end portion 2367, and/or the like. In some embodiments, the trailing tip capture mechanism 340 includes multiple loops to facilitate a staged release of the trailing end portion 2367 from the inner catheter assembly 310, as described in detail above.

A release wire 2341 can be inserted through the loop 2345 to releasably secure the loop 2345 to the trailing end portion 2367 of the aortic repair device 2350. The release wire 2341 can be pulled proximally to release the trailing end portion 2367 from the trailing tip capture mechanism 340. In some embodiments, the release wire 2341 is further coupled to the leading tip capture mechanism 342 (e.g., a loop thereof extending through the leading end portion 2361) such that actuation (e.g., withdrawal) of the release wire 2341 is configured to release both the leading end portion 2361 from the leading tip capture mechanism 342 and, upon further actuation (e.g., proximal retraction), the trailing end portion 2367 from the trailing tip capture mechanism 340. That is, the same release wire can be used to release all or a portion (e.g., a stage) of the leading and trailing end portions 2361, 2367.

In some aspects of the present technology, the trailing tip capture mechanism 340 can place the aortic repair device 2350 under tension during deployment from the outer catheter 302 (FIG. 3A) to inhibit or even prevent trailing ones of the stents 2376 from springing forward distally in the direction of arrow D as, for example, leading ones of the stents 2376 are released from the outer catheter 302 and expand within an aorta. Such a springing motion could cause the stents 2376 to jump or spring forward and contact (e.g., crash into) adjacent ones of the stents 2376, thereby reducing an overall length L of the aortic repair device 2350. Accordingly, the trailing tip capture mechanism 340 can bias or pull the aortic repair device 2350 proximally in the direction of arrow P to maintain the length L of the aortic repair device 2350 after deployment.

In some embodiments, a trailing tip capture mechanism comprises a mechanical connector secured to a trailing end portion of an aortic repair device via one or more release wires, as described in detail above with reference to FIGS. 15-20B and 41A-42B. More specifically, referring to FIG. 3C, the trailing tip capture mechanism 340 can include some features that are at least generally similar in structure and function, or identical in structure and function, to the corresponding features of the leading tip capture mechanism 1542, the leading tip capture mechanism 1642, and/or the leading tip capture mechanism 4142 described in detail above with reference to FIGS. 15-17D, 41A, and 41B, and can operate in a generally similar or identical manner to the leading tip capture mechanism 1542, the leading tip capture mechanism 1642, and/or the leading tip capture mechanism 4142. For example, in the illustrated embodiment the trailing tip capture mechanism 340 includes a body 349 having a barbell-like shape defining a recess 346 configured to releasably receive and secure one or more apices (e.g., trailing apices) of the trailing stent 376b of the aortic repair device 350. The body 349 can define one or more lumens for receiving corresponding ones of the release wires 341. The release wires 341 can extend over corresponding ones of the apices of the trailing stent 376b within the recess 346 to secure the trailing end portion 367 of the aortic repair device 350 thereto and can be removed from corresponding ones of the lumens to release and deploy the trailing end portion 367 of the aortic repair device 350. In some embodiments, one or more of the release wires 341 extending through the trailing tip capture mechanism 340 can also extend through the leading tip capture mechanism 342 such that the same one(s) of the release wires 341 can be used to release all or a portion (e.g., a stage) of the leading and trailing end portions 361, 367. The trailing tip capture mechanism 340 illustrated in FIG. 3C can place the aortic repair device 350 under tension during deployment from the outer catheter 302 (FIG. 3A) to inhibit or even prevent trailing ones of the stents 376 from springing forward distally and crashing into adjacent ones of the stents 2376, thereby maintaining an overall length of the aortic repair device 350 after deployment from the outer catheter 302.

Many of the embodiments described above include the use of one or more release wires for releasing a tip capture mechanism. Although certain drawings may illustrate the release wires extending past the distal end of the corresponding delivery system, one skilled in the art will appreciate that these wires can instead terminate at, on, and/or within the distal end portion of the delivery system (e.g., at, on, and/or within a tip member of the delivery system). For example, the release wires may extend past the distal end of the system during an assembly process and then subsequently be retracted into the tip member before the system is prepared for use.

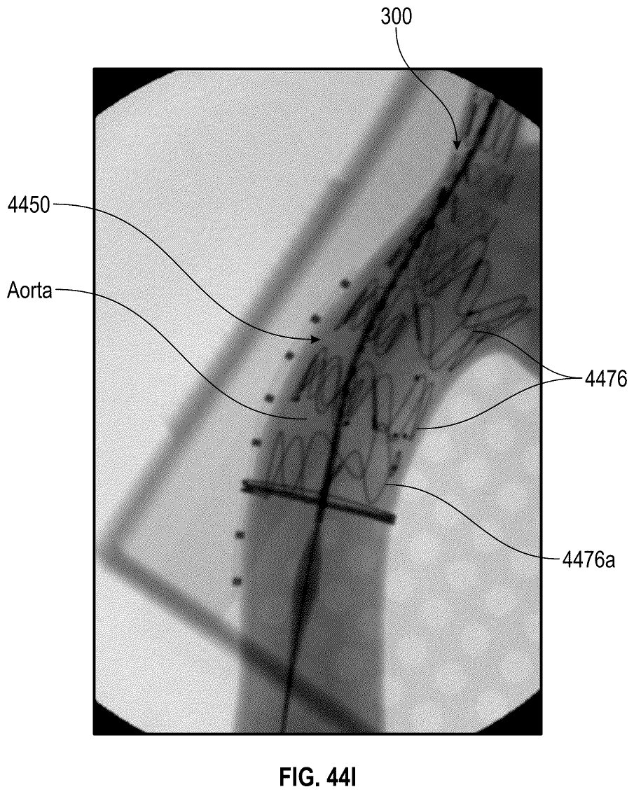

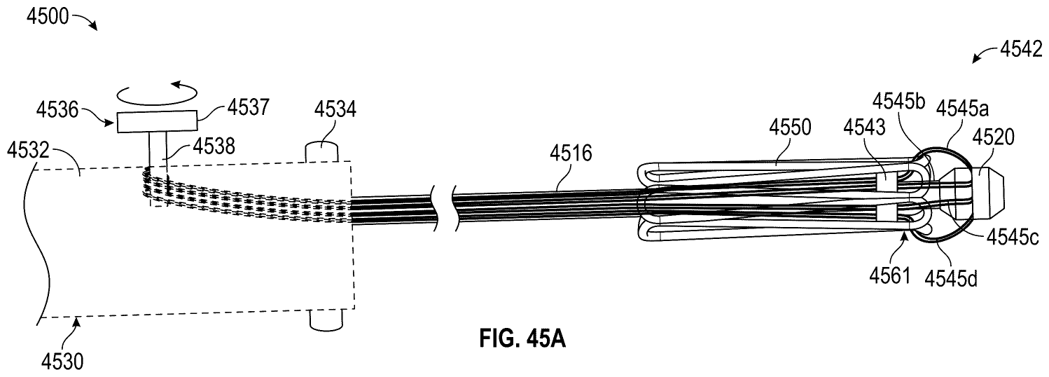

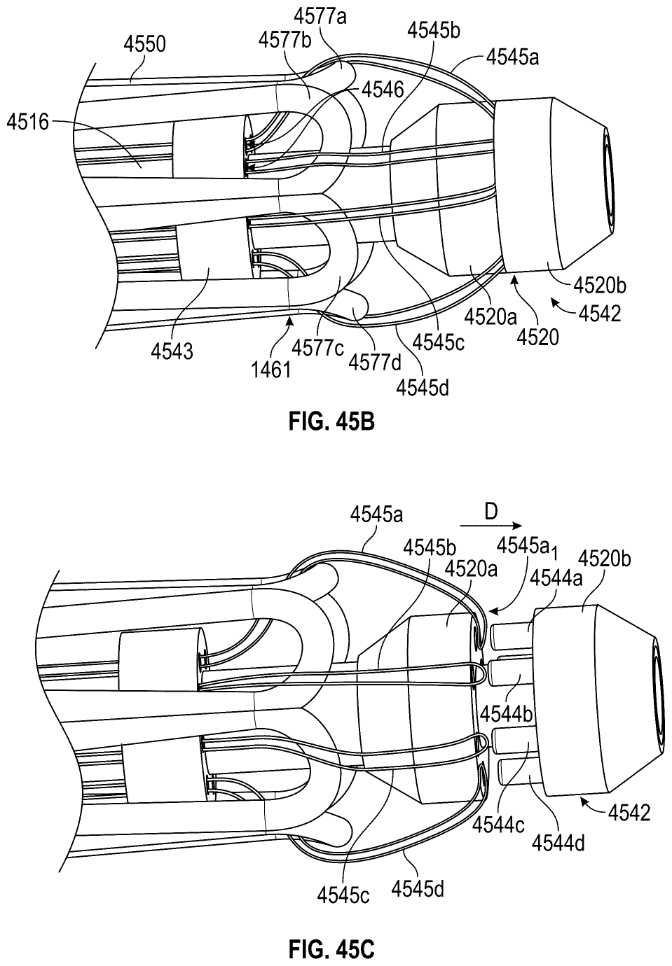

FIGS. 45A-45C illustrate a delivery system 4500 having a tip capture mechanism 4542 configured in accordance with some embodiments of the present technology. More specifically, FIG. 45A is a side view of the delivery system 4500 with an aortic repair device 4550 secured thereto in a collapsed (e.g., delivery) configuration, FIG. 45B is an enlarged view of a distal end region of the delivery system 4500 showing the tip capture mechanism 4542 in a first (secured) configuration, and FIG. 45C is an enlarged view of the distal end region of the delivery system 4500 showing the tip capture mechanism 4542 in a second (e.g., partially released) configuration. The delivery system 4500 and the aortic repair device 4550 can include various features that are generally similar in structure and function to features of any of the other delivery systems and aortic repair devices described throughout this Detailed Description, except where the context clearly dictates otherwise.

As shown in FIG. 45A, the delivery system 4500 includes a handle 4530 and an inner catheter 4516 extending from the handle 4530. The delivery system 4500 can also include a pusher catheter and an outer catheter, similar to the pusher catheter 312 and the outer catheter 302 of the delivery system 300 described with reference to FIGS. 3A-3G (not illustrated in FIGS. 45A-45C for clarity). In these embodiments, the aortic repair device 4550 can be secured around the inner catheter 4516 between a tip member 4520 and the pusher catheter (not shown). The handle 4530 and the inner catheter 4516 can also be generally similar to the handle 330 and the inner catheter 316, respectively, of the delivery system 300 of FIGS. 3A-3G. However, the handle 4530 of the delivery system 4500 of FIGS. 45A-45C further includes a tip capture driver 4534 (which can also be referred to as a tip capture drive mechanism, a tip capture release, a tip capture actuator, or the like) and a tip capture adjuster 4536 (which can also be referred to as a tip cap adjustment mechanism or the like). The tip capture driver 4534 can be a rotatable knob that is rotatably coupled to a housing 4532 of the handle 4530 or a portion thereof. In other embodiments, the tip capture driver 4534 can include a push button, a toggle button, a sliding tab, or other suitable actuator, instead of and/or in addition to the rotatable knob. The tip capture adjuster 4536 can include a head 4537 (which can also be referred to as an adjustment actuator, a handle, gripper, or the like) positioned external to the housing 4532 of the handle 4530 and/or otherwise positioned such that it is accessible by a user (e.g., a clinician), and a shaft 4538 extending from the head 4537 into an interior of the housing 4532 where it operably couples to components of the tip capture mechanism 4520. The head 4537 and the shaft 4538 can be fixedly coupled (e.g., integrally manufactured and/or coupled together via other techniques) such that the head 4537 and the shaft 4538 can rotate together relative to the housing 4532.

The tip capture mechanism 4542 can include a plurality of suture loops 4545 (“the loops 4545”; individually identified as a first loop 4545a, a second loop 4545b, a third loop 4545c, and a fourth loop 4545d) that can be composed of threads, fibers, cables, wires, and/or other engagement structures for retaining portions of the device 4550. The loops 4545 can extend from the tip member 4520 at the distal portion of the delivery system 4500 toward the handle 4530. In some embodiments, the loops 4545 can extend along an outer surface of the inner catheter 4516, e.g., between the inner catheter 4516 and the pusher catheter (not shown), between the pusher catheter and the outer catheter (not shown), and/or within individual lumens along the length of one of the catheters of the delivery system 4500. A proximal end of each loop 4545 can be coupled to (e.g., looped around, adhered to, etc.) the shaft 4538 of the tip capture adjuster 4536 within the housing 4532 of the handle 4530. As described in greater detail below with reference to FIGS. 45B and 45C, a distal end of the loops 4545 can be releasably coupled to (e.g., looped around, extended through, releasably connected to) various retention features in the tip member 4520. The tip capture mechanism 4542 can further include a collar 4543 positioned around the inner catheter 4516 at or near the tip member 4520, e.g., adjacent a leading (e.g., proximal) end 4561 of the aortic repair device 4550.

As shown in FIG. 45B, the loops 4545 can be threaded through openings near the leading end portion 1461 of the aortic repair device 1450 and releasably held in place by the tip member 4520. For example, the first loop 4545a can extend partially around and over a first apex 4577a of the aortic repair device 1450, the second loop 4545b can extend over a second apex 4577b of the aortic repair device 1450, the third loop 4545c can extend over a third apex 4577c of the aortic repair device 1450, and the fourth loop 4545d can extend over a fourth apex 4577d of the aortic repair device 1450. The tip capture mechanism 4542 can include additional loops that extend over other apices and/or other portions of the aortic repair device 4550. Accordingly, the tip capture mechanism 4542 can include the same number of loops 4545 as the number of apices 4577 at the leading end 4561 of the aortic repair device 4550. In other embodiments, the tip capture mechanism 4542 can include fewer loops 4545 than the number of apices 4577. In such embodiments, a single loop 4545 may be threaded through multiple apices 4577 and/or certain apices 4577 may not have a corresponding loop 4545. The loops 4545 can extend to the tip member 4520 and be positioned between a tip member first portion 4520a and a tip member second portion 4520b such that they are releasably held in place. The loops 4545 can also extend through corresponding channels or lumens 4546 of the collar 4543. The collar 4543 and the channel 4546 can retain the loops proximate to the inner catheter 4516 to maintain their spaced apart positions, prevent or reduce the likelihood of the loops 4545 entangling, and/or migrating away from their corresponding apex 4577.

Referring FIG. 45C, which illustrates the tip member second portion 4520b disconnected from the tip member first portion 4520a, the tip capture mechanism 4542 can include a plurality of retention features 4544, shown as pins (“the pins 4544”; individually identified as a first pin 4544a, a second pin 4544b, a third pin 4544c, and a fourth pin 4544d). The pins 4544 can extend from the tip member second portion 4520b and be sized and shaped to be slidably received within corresponding openings in the tip member first portion 4520a when the tip member first portion 4520a and the tip member second portion 4520b are coupled together in the configuration shown in FIG. 45B. In the illustrated embodiment, each of the pins 4544 have the same length. In other embodiments, however, some or all of the pins 4544 can have different lengths.

Individual loops 4545 can be retained within the tip member 4520 via corresponding individual pins 4544. For example, a distal end 4545a1 of the first loop 4545a can be looped around or otherwise coupled to the first pin 4544a. Distal ends (not labeled) of the other loops 4545 can similarly be looped around or otherwise coupled to the other pins 4544. When the tip member first portion 4520a is coupled to the tip member second portion 4520b (FIG. 45B) and the pins 4544 are received within the corresponding openings in the tip member first portion 4520a, the loops 4545 are retained within the tip member 4520 via the pins 4544. However, when the tip member second portion 4520a is decoupled from the tip member first portion 4520b (FIG. 45C), the loops 4545 can be released from the tip member 4520.

In operation, and referring collectively to FIGS. 45A-45C, the tip capture mechanism 4542 can facilitate a staged release of the aortic repair device 4550 from the delivery system 4500. For example, after the outer catheter (not shown) is withdrawn from over the aortic repair device 4550 as previously described, the loops 4545 can hold (e.g., capture) the leading end portion 4561 in a constrained (e.g., unexpanded or partially expanded) position, similar to the tip capture mechanisms previously described. Of note, the degree to which the loops 4545 constrain the leading end portion 4561 is adjustable. For example, a user can increase and decrease the tension on the loops 4545 by rotating the tip capture adjuster 4536 (FIG. 45A) in the clockwise and counterclockwise direction, respectively, which thereby adjusts the degree to which the loops 4545 constrain the aortic repair device 4550. For example, as the tip capture adjuster 4536 rotates clockwise, the loops 4545 are wound around the shaft 4538, which decreases the amount of slack in the loops 4545 and increases the amount of constraint provided by the loops 4545 (e.g., the leading end portion 4561 radially contracts). As the tip capture adjuster 4536 is rotated counterclockwise, the loops are unwound from around the shaft 4538, which increases the amount of slack in the loops 4545 and consequently decreases the amount of constraint provided by the loops 4545 (e.g., the leading end portion 4561 radially expands). The tip capture adjuster 4536 can alternatively be configured such that rotating the tip capture adjuster 4536 clockwise unwinds the loops 4545 from around the shaft 4538 and rotating the tip capture adjuster 4536 counterclockwise winds the loops 4545 around the shaft 4538. In some embodiments, the top capture adjuster 4536 can include different or additional mechanisms to adjust the tension on the loops 4545.

The adjustable range of the tip capture mechanism 4542 can be set based on the length of the loops 4545 and/or the rotatable range of the tip capture adjuster 4536. In some embodiments, the adjustable range extends between a fully (or near fully) constrained configuration and a fully (or near fully) expanded configuration, such that the leading end portion 4561 can be selectively and reversibly transitioned between the fully (or near fully) constrained configuration and the fully (or near fully) expanded configuration simply by rotating the tip capture adjuster 4536. In other embodiments, the adjustable range may be less than between the fully constrained configuration and the fully expanded configuration. In some embodiments, the tip capture adjuster 4536 can be controlled such that the leading end portion 4561 can occupy any configuration within the adjustable range (e.g., an infinite number of configurations between the fully constrained configuration and the fully expanded configuration). In other embodiments, the tip capture adjuster 4536 can be adjusted through a set number of incremental configurations between the fully constrained configuration and the fully expanded configuration.

In some embodiments, the tip capture adjuster 4536 simultaneously adjusts the degree of expansion of each of the apices 4577 of the leading end portion 4561 because each of the loops 4545 is wound around the shaft 4538. Accordingly, when the tip capture adjuster 4536 is rotated, each of the loops 4545 are wound around or unwound from the shaft 4538 at the same rate, and the same amount of slack is removed from or provided to each of the loops 4545. In other embodiments, however, the tip capture mechanism 4542 can include a plurality tip capture adjusters 4536 (e.g., two, three, four, five, six, seven, or more). In such embodiments, individual loops 4545 can be coupled to individual tip capture adjusters 4536, such that the amount of constraint for each of the apices 4577 can be individually controlled by rotating individual ones of the tip capture adjusters 4536. In some embodiments, some of the loops 4545 may be coupled to a fixed portion of the delivery system 4500 instead of the tip capture adjuster 4536 such that some of the loops 4545 are not adjustable.

To release the aortic repair device 4550 from the tip capture mechanism 4542, the tip capture driver 4534 (FIG. 45A) can be actuated to disconnect the tip member second portion 4520b from the tip member first portion 4520a. For example, rotating the tip capture driver 4534 can drive the tip member second portion 4520b distally relative to the tip member first portion 4520a (e.g., in a direction D labeled in FIG. 45C). Alternatively, rotating the tip capture driver 4534 can drive the tip member first portion 4520a proximally relative to the tip member second portion 4520b, e.g., in a direction opposite of the direction D. In either embodiment, rotating the tip capture driver 4534 moves the tip member first portion 4520a and the tip member second portion 4520b away from one another. The tip capture driver 4534 can be rotated until the pins 4544 are withdrawn from the openings in the tip capture first portion 4520a. This releases the loops 4545 and enables the aortic repair device 4550 to fully expand.

As described above, in some embodiments the pins 4544 can have different lengths. In such embodiments, the individual loops 4545 can be released from the corresponding pins 4544 at different stages of a procedure. For example, a first subset of the loops 4545 (and the corresponding apices 4577) retained by relatively shorter pins 4544 will be released before a second subset of the loops 4545 (and apices 4577) retained by relatively longer pins. The pins 4544 can therefore be selectively sized based on a desired order of release from the tip capture mechanism 4542 for different apices 4577. This allows the tip capture mechanism 4542 to provide for a staged release of the aortic repair device 4550 that preferentially releases one loop 4545 or a subset of loops 4545 before other loops 4545, meaning that one portion or side of the aortic repair device 4550 can release and expand before another portion (e.g., the apices 4577 proximate to the lesser curve of the aorta can be released before the apices 4577 near the greater curve, or vice versa).

In some embodiments, the tip capture driver 4534 can be integrated with the tip capture adjuster 4536. For example, in some embodiments pressing the tip capture adjuster 4536 into the housing 4532 (or pulling the tip capture adjuster 4536 further out of the housing 4532) can drive the tip member second portion 4520b away from the tip member first portion 4520a, or vice versa, to release the loops 4545. In such embodiments, the rotatable knob 4534 can be omitted.

### IV. SELECTED EMBODIMENTS OF POSITIONING MECHANISMS FOR DELIVERY SYSTEMS FOR IMPLANTING AORTIC REPAIR DEVICES WITH IMPROVED SQUARENESS WITHIN A VESSEL

Referring to FIG. 4A, the aortic repair device 350 defines a treatment zone within the aorta and routes blood distally past treatment zone. The treatment zone can be adjacent a dissection within the aorta, such as an acute Type A dissection. Moreover, the leading end portion 361 of the aortic repair device 350 is positioned distal to the left coronary artery such that the left coronary artery is not covered/blocked by the aortic repair device 350. In some aspects of the present technology, it is beneficial to implant the aortic repair device 350 within the aorta with the leading end portion 361 squarely positioned within the aorta—for example, with the leading end portion 361 extending generally orthogonal to the inner wall of the aorta—rather than tilted within the aorta to maximize the length of the treatment zone and/or to ensure that the aortic repair device 350 does not block the distal most coronary artery, which is typically the left coronary artery.

Referring to FIG. 4C, the leading end portion 451 of the spanning member 452 defines a scaling zone within the first base member 360a and the trailing end portion 453 of the spanning member 452 defines a sealing zone within the second base member 360b. In some aspects of the present technology, it is beneficial to implant the spanning member 452 within the aorta with the leading end portion 451 squarely positioned within the first base member 360a and the trailing end portion 453 squarely positioned within the second base member 360b—for example, with the leading and trailing end portions 451, 453 extending generally orthogonal to an inner wall of the first base member 360a and an inner wall of second base member 360b, respectively-rather than tilted within the first and second base members 360a-b to maximize a length of the sealing zones.

FIGS. 24-34E and 44A-44I illustrate different positioning mechanisms of a delivery system for improving such squareness (e.g., of a base member within an aorta and/or of a spanning member within a base member) in accordance with embodiments of the present technology. The various mechanisms for improving squareness can be incorporated into and used in any of the delivery systems described herein. Moreover, the various aortic repair devices described with reference to FIGS. 24-34E and 44A-44I can include some features that are at least generally similar in structure and function, or identical in structure and function, to one another and/or to the corresponding features of any of the aortic repair devices described in detail above with reference to FIGS. 3A-23, and can operate in a generally similar or identical manner to one another and/or to the aortic repair devices of FIGS. 3A-23. Moreover, one or more of the different features of the positioning mechanisms of the present technology can be combined and/or omitted.

FIG. 24 is a perspective view of a distal portion of the delivery system 300 of FIGS. 3A-3G secured to an aortic repair device 2450 in accordance with embodiments of the present technology. In the illustrated embodiment, the aortic repair device 2450 is shown deployed and expanded from the outer catheter 302 (FIG. 3A) of the delivery system 300. The aortic repair device 2450 can be a spanning member including a plurality of stents 2476 coupled to a graft material 2478. The aortic repair device 2450 can include a leading end portion 2461 releasably secured to the leading tip capture mechanism 342 and a trailing end portion 2467 releasably secured to the trailing tip capture mechanism 340.

Referring to FIGS. 3E, 3F, and 24, retracting the outer catheter 302 to deploy the aortic repair device 2450 within an aorta can put the aortic repair device 2450 under tension as, for example, the outer catheter 302 frictionally engages the portion of the aortic repair device 2450 compressed therein. Such tension can reduce the conformance of the aortic repair device 2450 to the aorta. For example, the aortic repair device 2450 can have (i) a first side portion 2456 configured to conform to an outer portion (e.g., an outer curved portion) of an interior wall of the aorta having a greater curvature (e.g., greater radius of curvature, longer length), and (ii) a second side portion 2457 of the opposite to the first side portion 2456 that is configured to conform to an inner portion (e.g., an inner curved portion) of the interior wall of the aorta having a lesser curvature (e.g., a smaller radius of curvature, shorter length). Accordingly, once the aortic repair device 2450 is deployed, it can be beneficial for the first side portion 2456 to have a longer length than the second side portion 2457. However, tension from retraction of the outer catheter 302 can stretch the aortic repair device 2450 to a maximum length before/during deployment such that such differential length is not achieved.

Accordingly, in some embodiments the pusher catheter 312 can be advanced distally within the outer catheter 302 relative to the inner catheter 316 when the delivery system 300 is in the delivery position (FIG. 3E) to compress the aortic repair device 2450 within the outer catheter 302. For example, the pusher catheter 312 can directly contact the aortic repair device 2450 (e.g., a trailing stent thereof) or push the trailing tip capture mechanism 340 to compress the aortic repair device 2450 while the leading end portion 2461 remains fixed in position to the inner catheter 316 via the leading tip capture mechanism 342. With the aortic repair device 2450 pre-compressed within the outer catheter 302, retraction of the outer catheter 302 to the deployed position can increase tension in the aortic repair device 2450 without fully stretching the aortic repair device 2450 to its maximum length such that the second side portion 2457 can remain partially compressed to conform to the inner portion of the aorta and the first side portion 2456 can fully expand and lengthen to conform to the outer portion of the aorta. The amount of compression can be about 5%, about 10%, or more than 10% of a length of the aortic repair device 2450. In some embodiments, increasing a distance between the stents 2476 can allow for a greater amount of compression.

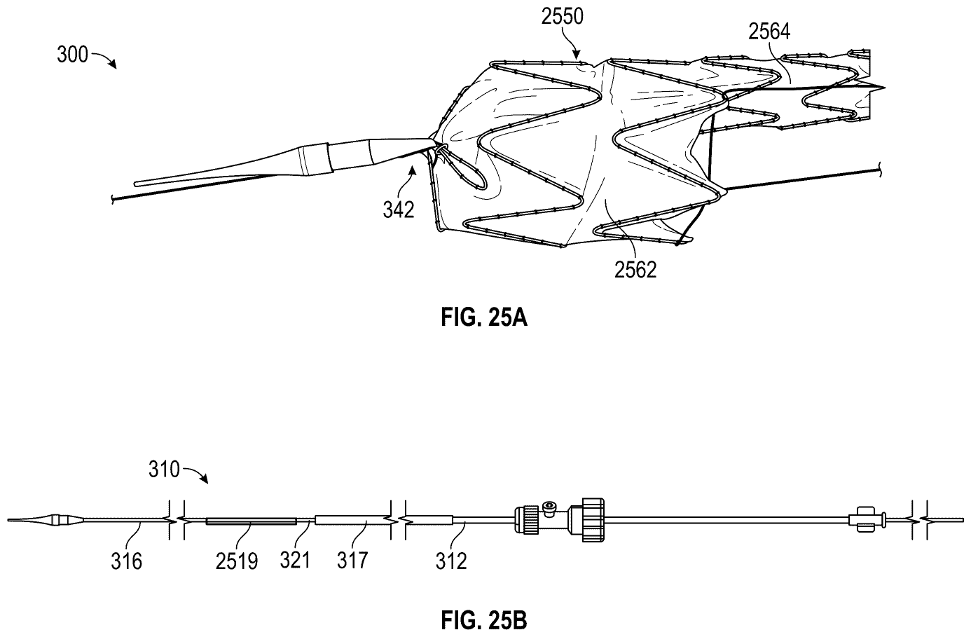

FIG. 25A is a perspective view of a distal portion of the delivery system 300 of FIGS. 3A-3G secured to an aortic repair device 2550 in accordance with embodiments of the present technology. In the illustrated embodiment, the aortic repair device 2450 is shown deployed and expanded from the outer catheter 302 (FIG. 3A) of the delivery system 300. The aortic repair device 2550 can be a base member including a body 2562 releasably secured to the leading tip capture mechanism 342 and a leg 2564. The leg 2564 can be secured to the trailing tip capture mechanism 340 (FIGS. 3B and 3C).

FIG. 25B is a side view of the inner catheter assembly 310 of the delivery system 300 of FIGS. 3A-3G in accordance with embodiments of the present technology. In the illustrated embodiment, the inner catheter assembly 310 further includes a pusher member 2519 extending distally adjacent the inner catheter 316. The pusher member 2519 can comprise an extension of the stopper member 321, or a separate member that is coupled to the pusher catheter 312. Referring to FIGS. 3E, 3F, 25A, and 25B, when the aortic repair device 2550 is compressed within the outer catheter 302 in the delivery position (FIG. 3E), the pusher catheter 312 (e.g., the enlarged portion 317) can engage the leg 2564 of the aortic repair device 2550, and the pusher member 2519 can engage the body 2562. Accordingly, the pusher catheter 312 can be advanced distally within the outer catheter 302 relative to the inner catheter 316 to compress both the leg 2564 and the body 2562 of the aortic repair device 2550. In some aspects of the present technology, such compression can improve conformance of the aortic repair device 2550 within an aorta when the aortic repair device 2550 is deployed therein, as described in detail above with reference to FIG. 24.

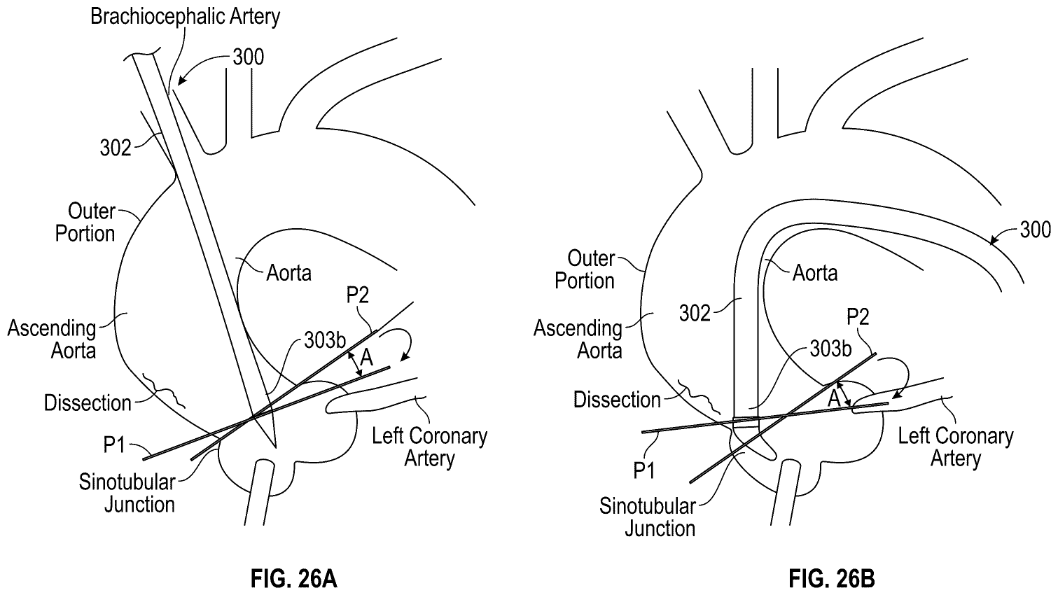

FIGS. 26A and 26B are side views of the delivery system 300 of FIGS. 3A-3G positioned at least partially within an aorta and configured to deploy an aortic repair device in accordance with embodiments of the present technology. Referring to FIG. 26A, the delivery system 300 is inserted through the brachiocephalic artery such that the leading end portion 303b of the outer catheter 302 is positioned within the ascending aorta proximate the sinotubular junction. Referring to FIG. 26B, the delivery system 300 is inserted through the aorta such that the leading end portion 303b of the outer catheter 302 is positioned within the ascending aorta proximate the sinotubular junction. Referring to FIGS. 26A and 26B, the tortuous/angled anatomy of the aorta and its branch vessels can cause the leading end portion 303b of the outer catheter 302 to be aligned along a first plane P1. The first plane P1 can generally correspond to a subsequent deployment position of the leading end portion of the aortic repair device after deployment from the outer catheter 302. That is, the leading end portion of the aortic repair device can be positioned along the first plane P1 after deployment from the outer catheter 302.

In the illustrated embodiment, the first plane P1 is at an angle A (e.g., a negative angle) to a second plane P2 of the sinotubular junction. In some aspects of the present technology, it can be beneficial to improve the squareness of the delivery system 300 within the aorta by minimizing the angle A or even making the angle A positive to, for example, (i) inhibit or even prevent the aortic repair device from covering/blocking the left coronary artery after deployment and/or (ii) maximize a sealing/treatment zone of the aortic repair device adjacent an outer portion of the aorta (e.g., including a dissection). Accordingly, in some embodiments a delivery system in accordance with embodiments of the present technology can include one or more mechanisms for steering the delivery system 300 within the aorta to change the angle A and/or to otherwise improve the squareness of the aortic repair device after deployment.

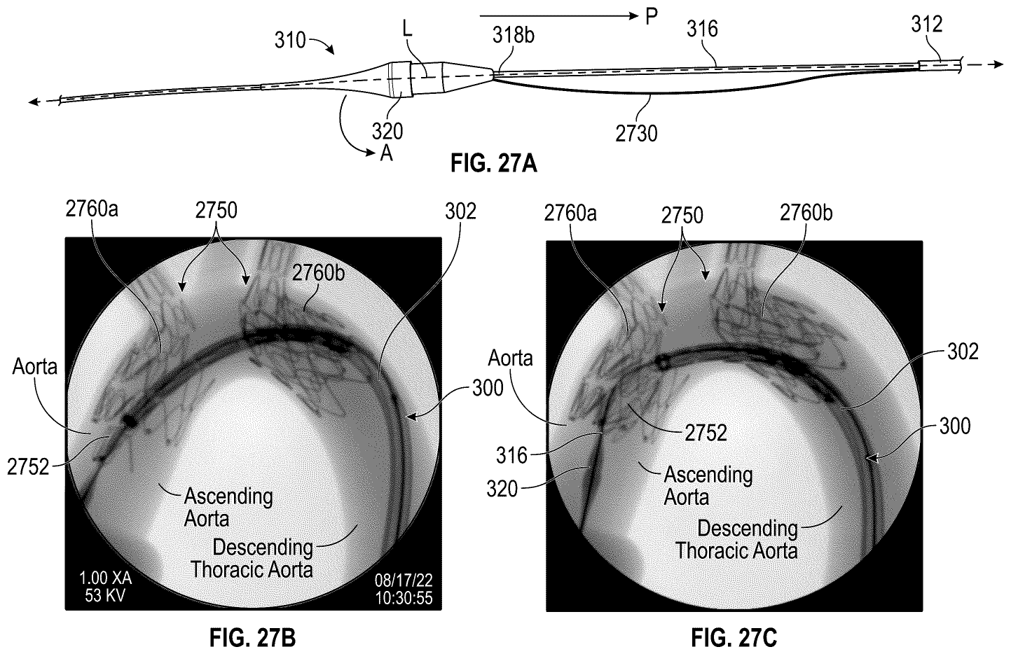

FIG. 27A, for example, is a perspective side view of a distal portion of the inner catheter assembly 310 of the delivery system 300 of FIGS. 3A-3G in accordance with embodiments of the present technology. In the illustrated embodiment, the inner catheter assembly 310 further includes a tendon 2730 coupled to the tip member 320 and/or the leading end portion 318b of the inner catheter 316. The tendon 2730 can be routed proximally to the handle 330 (FIG. 3A) and/or another component of the delivery system 300. In some embodiments, the tendon 2730 extends proximally through the pusher catheter 312. The tendon 2730 can comprise a metal wire, a suture, and/or the like. The tendon 2730 can be pulled proximally in the direction of arrow P (e.g., via a component of the handle 330) to pull the tip member 320 to deflect/flex away from a longitudinal axis L of the inner catheter assembly 310 as indicated by arrow A. Accordingly, the tendon 2730 provides for steerability of the tip member 320 of the inner catheter assembly 310. The amount of deflection of the tip member 320 can be controlled by retracting the tendon 2730 more or less. In some embodiments, the inner catheter 316 comprises a hypotube (e.g., a laser cut hypotube) shaped to permit deflection along only a single plane or to match the deflection to the anatomy of a patient. In some embodiments, when the inner catheter assembly 310 is positioned within the outer catheter 302 (FIG. 3A), the deflection of the inner catheter assembly 310 can act to at least partially deflect the outer catheter 302 in a corresponding manner.

FIGS. 27B and 27C are side views (e.g., fluoroscopic images) of a first stage and a second stage, respectively, of a procedure to implant an aortic repair device 2750 within an aorta of a patient using the delivery system 300 of FIG. 27A in accordance with embodiments of the present technology. Referring to FIGS. 27B and 27C, the aortic repair device 2750 includes a first base member 2760a positioned at least partially within the ascending aorta and a second base member 2760b positioned at least partially within the descending thoracic aorta. The delivery system 300 can be configured to deploy a spanning member 2752 therebetween to provide a full-arch treatment, as described in detail above with reference to FIG. 4C.

Referring to FIG. 27B, in the first stage the delivery system 300 can be advanced through the second base member 2760b and at least partially through the first base member 2760a via an aortic approach with the spanning member 2752 compressed within the outer catheter 302. Referring to FIGS. 27A and 27C, in the second stage the tendon 2730 can be actuated to deflect the tip member 320 and the inner catheter 316 of the inner catheter assembly 310 to, for example, more squarely position a leading end portion of the spanning member 2752 within the first base member 2760a. The outer catheter 302 can then be retracted to deploy the spanning member 2752 within the first base member 2760a as shown in FIG. 27C. In some aspects of the present technology, such steering of the inner catheter assembly 310 and the spanning member 2752 coupled thereto can improve the conformance and seal between the first base member 2760a and the spanning member 2752 despite the tortuous/angled anatomy of the aorta.

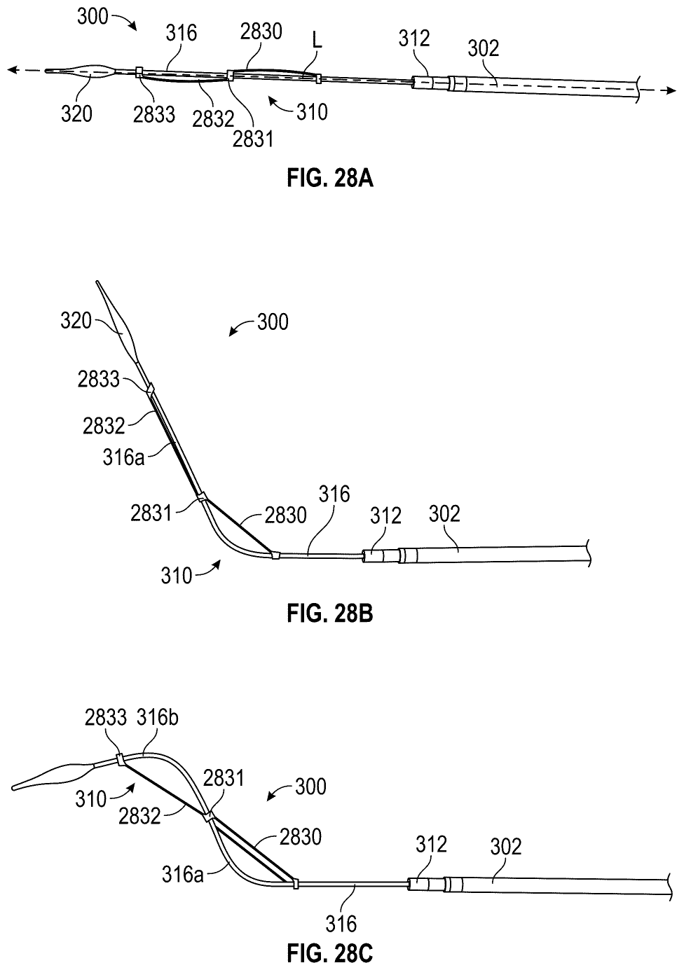

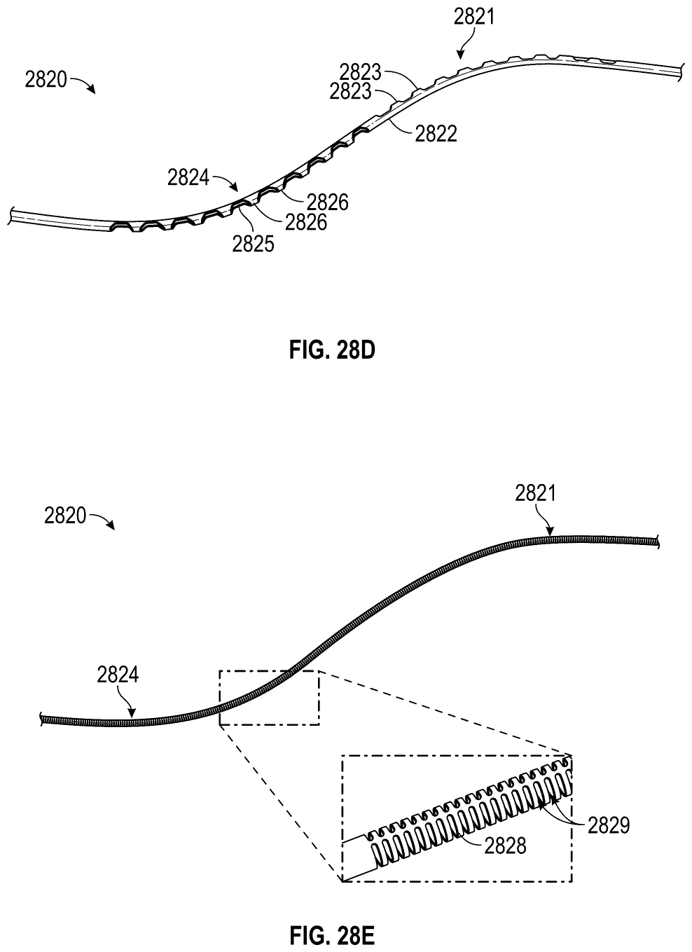

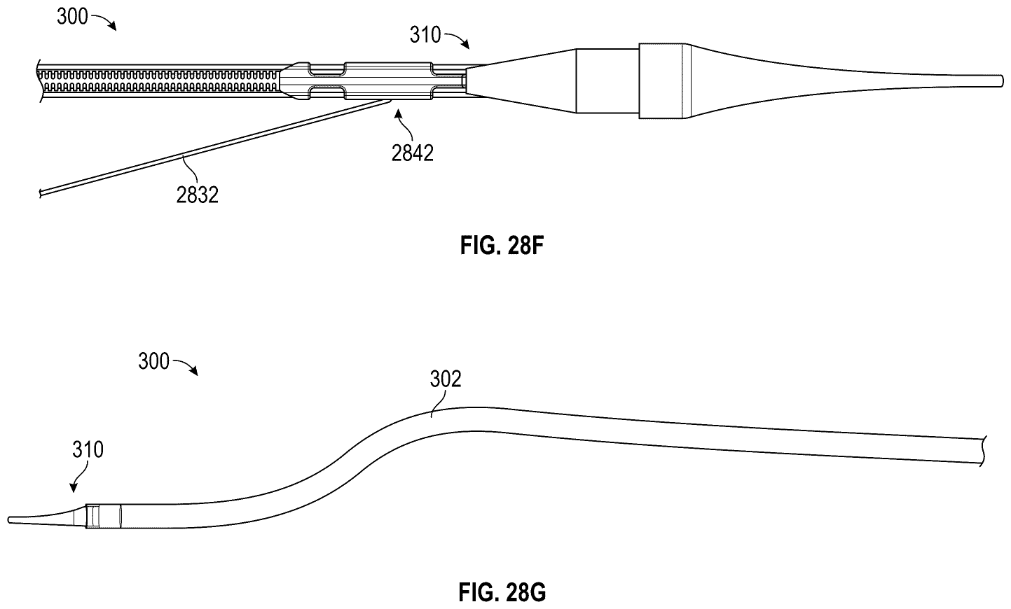

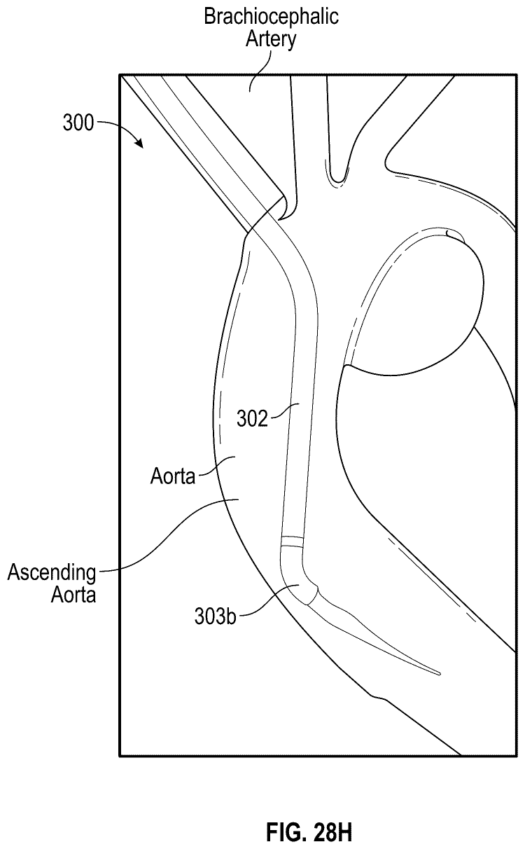

FIGS. 28A-28C are perspective side views of a distal portion of the inner catheter assembly 310 of the delivery system 300 of FIGS. 3A-3G in a first position, a second position, and a third position, respectively, in accordance with embodiments of the present technology. Referring to FIGS. 28A-28C, the inner catheter assembly 310 further includes (i) a first tendon 2830 coupled (e.g., fixed) to the inner catheter 316 via a first coupling member 2831 (e.g., a ring) and (ii) a second tendon 2832 coupled to the inner catheter 316 distal of the first tendon 2830 via a second coupling member 2833. The first and second tendons 2830, 2832 can be routed proximally to the handle 330 (FIG. 3A) and/or another component of the delivery system 300 through, for example, the pusher catheter 312 and/or the outer catheter 302. The first and second tendons 2830, 2832 can comprise metal wires, sutures, and/or the like. In other embodiments, the first and second tendons 2830, 2832 can be integrated into a single tendon.

Referring to FIG. 28A, the inner catheter assembly 310 is in a first position (e.g., an unflexed) in which the inner catheter 316 generally extends along a longitudinal axis L. Referring to FIG. 28B, the inner catheter assembly 310 is in a second position (e.g., a first flexed position) in which a first portion 316a of the inner catheter 316 and the tip member 320 are deflected away from the longitudinal axis L. To move the inner catheter assembly 310 to the second position, the first tendon 2730 can be pulled proximally (e.g., via a component of the handle 330 of FIG. 3A) to deflect/flex the first portion 316a of the inner catheter 316. Referring to FIG. 28C, the inner catheter assembly 310 is in a third position (e.g., a second flexed position) in which a second portion 316b of the inner catheter 316 and the tip member 320 are deflected away from the first portion 316a, for example, back toward the longitudinal axis L (FIG. 3A). Accordingly, the inner catheter 316 can have a generally S-like or Z-like shape in the third position. To move the inner catheter assembly 310 to the third position, the second tendon 2730 can be pulled proximally (e.g., via a component of the handle 330 of FIG. 3A) to deflect/flex the second portion 316b of the inner catheter 316.

In some embodiments, the inner catheter 316 comprises a hypotube (e.g., a laser cut hypotube) shaped to permit deflection of the first and second portions 316a-b of the inner catheter along only a single plane or to match the deflection to the anatomy of a patient. For example, FIG. 28D is a perspective side view of a sleeve 2820 (e.g., a portion) of the inner catheter 316 of FIGS. 28A-28C in accordance with embodiments of the present technology. In the illustrated embodiment, the sleeve 2820 is a hypotube having (i) a first region or portion 2821 comprising a first spine 2822 and a plurality of first ribs 2823 extending from the first spine 2822 and (ii) a second region or portion 2824 comprising a second spine 2825 and a plurality of second ribs 2826 extending from the second spine 2825. In some embodiments, the first portion 2821 is shaped to bend in a first direction and the second portion 2824 is shaped to bend in a second direction different than the first direction. For example, referring to FIGS. 28C and 28D, the first portion 2821 of the sleeve 2820 can be positioned in/along the first portion 316a of the inner catheter 316 and the second portion 2824 of the sleeve 2820 can be positioned in/along the second portion 316b of the inner catheter 316 to permit/constrain the inner catheter 316 to flex to the third position shown in FIG. 28C. The sleeve 2820 can be formed from nitinol, metal (e.g., stainless steel), plastic, and/or the like. In some embodiments, the first coupling member 2831 is positioned between the first portion 2821 and the second portion 2824 of the sleeve 2820, and the second coupling member 2833 is positioned distal of the second portion 2824 of the sleeve 2820. In some embodiments, the first coupling member 2831 and/or the second coupling member 2833 can be omitted and the first tendon 2830 and/or the second tendon 2832 can be directly welded to the inner catheter 316. In some aspects of the present technology welding the first and second tendons 2830, 2832 to the inner catheter 316 can minimize the size of the attachment points for the first and second tendons 2830, 2832, thereby preserving space for an aortic repair device coupled to the inner catheter 316 to compress.

FIG. 28E is a perspective side view of the sleeve 2820 (e.g., a portion) of the inner catheter 316 of FIGS. 28A-28C in accordance with additional embodiments of the present technology. In the illustrated embodiment, the sleeve 2820 is a hypotube including the first portion 2821 shaped or material is selectively removed to bend in a first direction and the second portion 2824 shaped or material is selectively removed to bend in the second direction different than the first direction. The sleeve 2820 (e.g., the first portion 2821 and/or the second portion 2824) can be formed via laser cutting and/or via an endmill to have ribs 2828 defining grooves or slots 2829 therebetween. The ribs 2828 can be spaced closer together than the first ribs 2823 and/or the second ribs 2826 of FIG. 28D to, for example, promote global flexing of the first portion 2821 and/or the second portion 2824 (e.g., such that the first portion 2821 and/or the second portion 2824 assumes a greater radius of curvature when flexed).

In some embodiments, the second coupling member 2833 can be a portion of a leading tip capture mechanism of the delivery system 300. For example, FIG. 28F is a side view of a distal portion of the inner catheter assembly 310 of FIGS. 28A-28C including a leading tip capture mechanism 2842 in accordance with embodiments of the present technology. The leading tip capture mechanism 2842 can be similar or identical in structure and/or function to any of the leading tip capture mechanisms described in detail above, such as the leading tip capture mechanism 1542, the leading tip capture mechanism 1642, and/or the leading tip capture mechanism 4142 described in detail above with reference to FIGS. 15-17D, 41A, and 41B. In the illustrated embodiment, the tip capture mechanism 2842 is designed such that a single release wire can release more than one stage of tip capture. This tip capture mechanism 2842 includes a release wire that travels along a channel or through hole. There can be a series of windows or reliefs cut long the channel allowing for a new stage of tip capture to be tied down at each window. Retracting the release wire past the first window can release the first tip capture and continuing to retract the release wire past the second window can release another stage tip capture and so on. In addition, in the illustrated embodiment the second tendon 2832 is directly attached to the leading tip capture mechanism 2842 such that the leading tip capture mechanism 2842 functions as the second coupling member 2833.

Referring to FIGS. 28A-28F, when the inner catheter assembly 310 is positioned within the outer catheter 302 in the delivery position (FIG. 3E), the deflection of the inner catheter assembly 310 can act to at least partially deflect the outer catheter 302 in a corresponding manner. FIG. 28G, for example, is a perspective side view of the distal portion of the delivery system 300 of FIGS. 28A-28F with the inner catheter assembly 310 in the third position and positioned within the outer catheter 302 in accordance with embodiments of the present technology. In the illustrated embodiment, the deflection of the inner catheter assembly 310 acts to correspondingly deflect the outer catheter 302.

FIG. 28H is a side view of the delivery system 300 of FIGS. 28A-28G positioned at least partially within an aorta and configured to deploy an aortic repair device in accordance with embodiments of the present technology. In the illustrated embodiment, the delivery system 300 is inserted through the brachiocephalic artery such that the leading end portion 303b of the outer catheter 302 is positioned within the ascending aorta (e.g., proximate the sinotubular junction). In the illustrated embodiment, the delivery system 300 is in the third position. As shown, the curvature/deflection of the delivery system 300 in the third position can help position the leading end portion 303b generally toward the center of the aorta and square the leading end portion 303b within the aorta. More specifically, referring to FIGS. 28A-28C and 28H, the deflection of the first portion 316a of the inner catheter 316 can allow the delivery system 300 to traverse the angle from the brachiocephalic artery into the aorta, and the deflection of the second portion 316b of the inner catheter 316 can allow the delivery system 300 to traverse the curvature of the ascending aorta to center the delivery system 300 therein. FIG. 28H shows the delivery system 300 deflected by the first and second tendons 2830, 2832 prior to unsheathing, which can conform the aortic repair device to the vessel, thereby inhibiting or even preventing the aortic repair device from moving relative to the vessel wall and stressing the vessel.

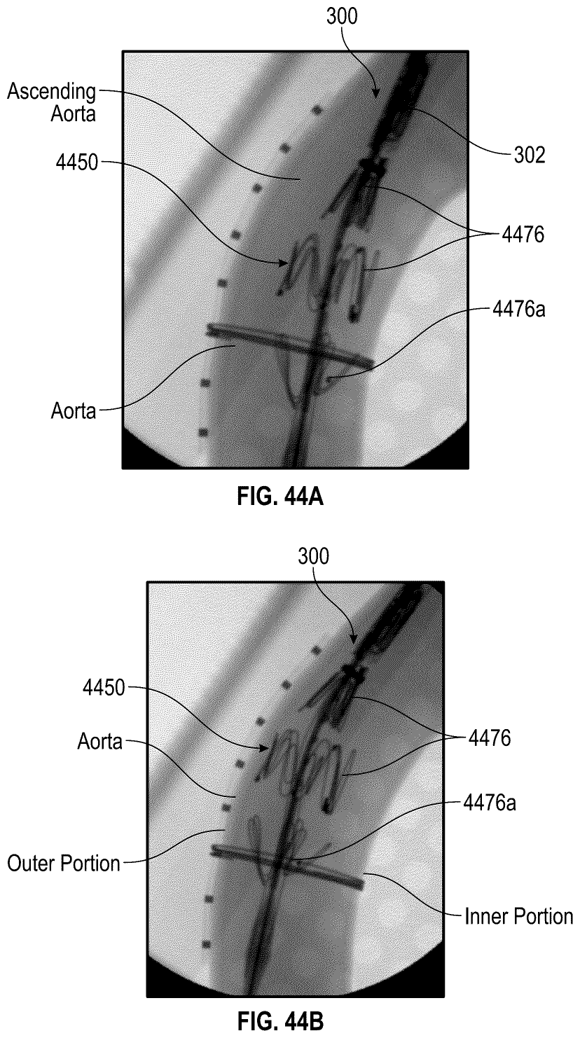

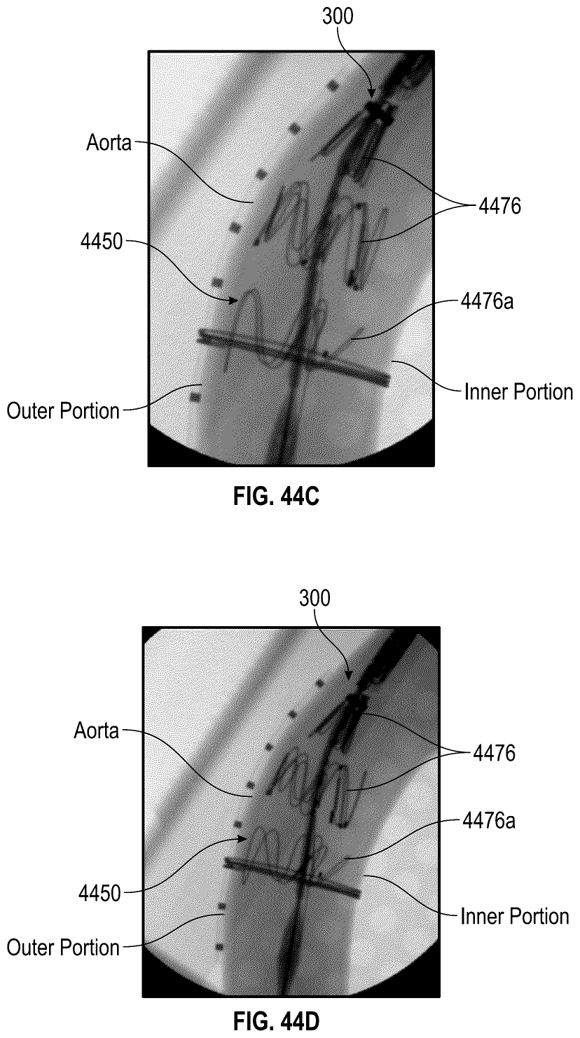

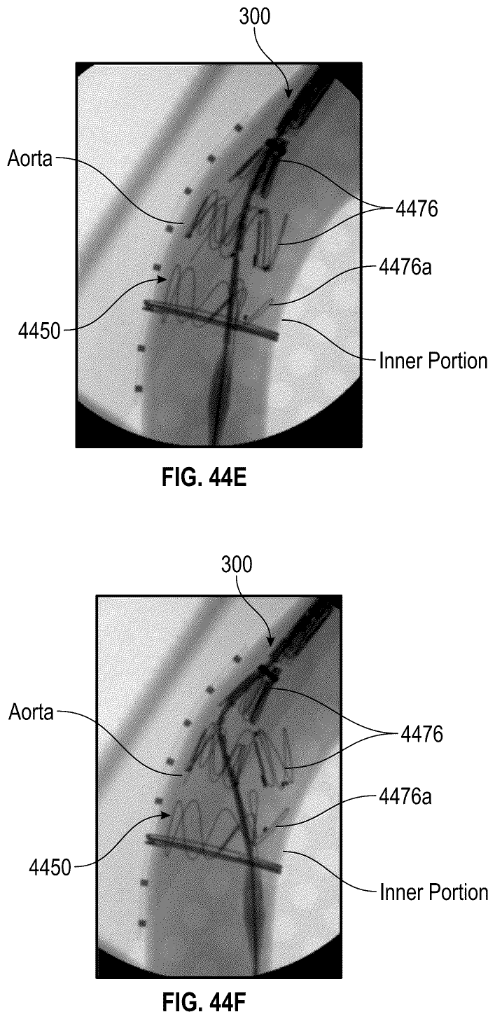

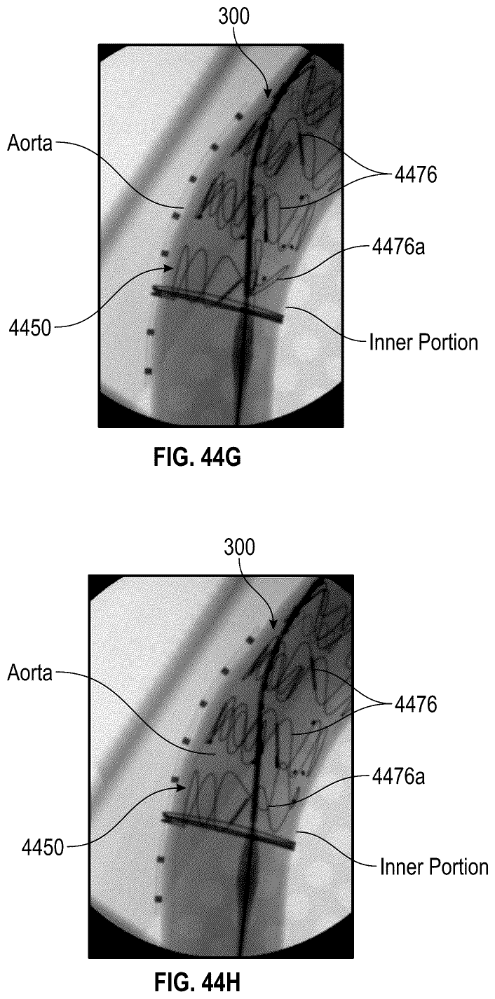

In some embodiments, an aortic repair device coupled to the delivery system of FIGS. 28A-28G can be fully or partially unsheathed from the outer catheter 302 during deployment, and the inner catheter 316 can be steered via the first and second tendons 2830, 2832 to facilitate positioning of the aortic repair device. For example, FIGS. 44A-44I are side views (e.g., fluoroscopic images) of different stages of a procedure to implant an aortic repair device 4450 within an aorta of a patient using the delivery system 300 of FIGS. 28A-28H in accordance with embodiments of the present technology. In the illustrated embodiment, the aortic repair device 4450 includes multiple stents 4476 (including an individually identified leading stent 4476a) secured to a graft material (obscured in FIGS. 44A-44I). The leading stent 4476a is configured to be releasably secured to the leading tip capture mechanism 342 (FIGS. 3B and 3C).

Referring to FIG. 44A, in a first stage the aortic repair device 4450 can be partially deployed from the outer catheter 302 at a target position within the aorta. For example, three of the stents 4476 are shown deployed from the outer catheter 302 within the ascending aorta. In the illustrated embodiment, the leading stent 4476a is cinched and secured to the leading tip capture mechanism 342 (FIGS. 3B and 3C).

Referring to FIG. 44B, in a second stage the first tendon 2830 (FIGS. 28A-28C) can be actuated (e.g., pulled, tensioned) to steer the aortic repair device 4450 toward an outer portion (e.g., an outer curved portion) of an interior wall of the aorta having a greater curvature (e.g., greater radius of curvature, longer length). The outer portion of the aorta is opposite an inner portion (e.g., an inner curved portion) of an interior wall of the aorta having a lesser curvature (e.g., a smaller radius of curvature, shorter length).

Referring to FIG. 44C, in a third stage the leading tip capture mechanism 342 (FIGS. 3B and 3C) can be actuated to release a first portion of the leading stent 4476a (e.g., in a first tip capture release stage) proximate the outer portion of the aorta. That is, some of the leading apices of the leading stent 4476a can be released from the leading tip capture mechanism 342 to allow the aortic repair device 4450 to expand adjacent the outer portion, while the leading tip capture mechanism 342 retains some of the leading apices adjacent the inner portion of the aorta.

Referring to FIG. 44D, in a fourth stage the first tendon 2830 (FIGS. 28A-28C) can be actuated (e.g., relaxed) to steer the aortic repair device 4450 toward a central portion of the aorta between the inner and outer portions. In some embodiments, releasing the first portion of the leading stent 4476a from the leading tip capture mechanism as shown in FIG. 44C can also help center the aortic repair device 4450 within the aorta as, for example, the leading stent 4476a expands and pushes against the outer portion of the aorta. Referring to FIG. 44E, in a fifth stage the leading tip capture mechanism 342 (FIGS. 3B and 3C) can be actuated to release a second portion of the leading stent 4476a (e.g., in a second tip capture release stage).

Referring to FIG. 44F, in a sixth stage the second tendon 2832 (FIGS. 28A-28C) can be actuated (e.g., pulled, tensioned) to steer the aortic repair device 4450 toward the inner portion of the aorta. In some embodiments, the first tendon 2830 (FIGS. 28A-28C) can be fully relaxed during actuation of the second tendon 2832.

Referring to FIG. 44G, in a seventh stage the second tendon 2832 (FIGS. 28A-28C) can be fully actuated (e.g., pulled, tensioned) to pull the aortic repair device 4450 square to the inner portion of the aorta and to induce a positive tilt of the leading stent 4476a against the inner portion. Referring to FIG. 44H, in an eighth stage the leading tip capture mechanism 342 (FIGS. 3B and 3C) can be actuated to release a third portion of the leading stent 4476a (e.g., in a third tip capture release stage). The third portion can be proximate/adjacent to the inner portion of the aorta.

At this stage, the leading stent 4476a can be fully released and decoupled from the leading tip capture mechanism 342. FIG. 44I shows the aortic repair device 4450 fully deployed from the delivery system 300. In some aspects of the present technology, selectively steering the aortic repair device 4450 via the first and second tendons 2830, 2832 (FIGS. 28A-28C) in the manner illustrated in FIGS. 44A-44I can improve the squareness of the aortic repair device 4450 after implantation while also inhibiting or even preventing the aortic repair device 4450 from covering/blocking the left coronary artery.

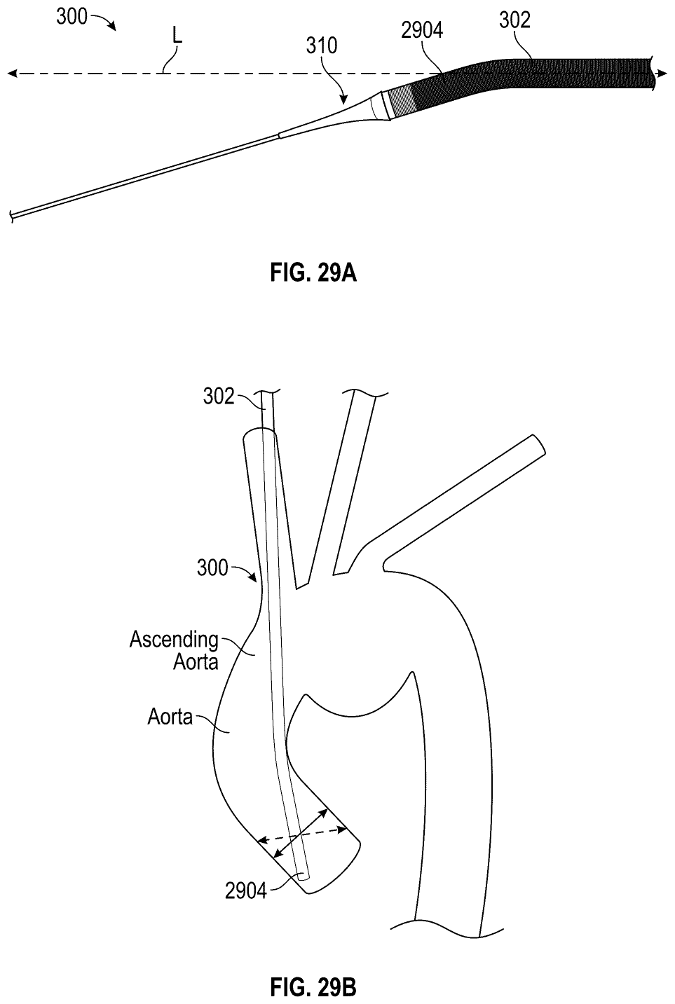

FIG. 29A is a perspective side view of a distal portion of the delivery system 300 of FIGS. 3A-3G in accordance with embodiments of the present technology. In the illustrated embodiment, the outer catheter 302 includes a leading portion 2904 shaped to deflect/extend away from a longitudinal axis L of the delivery system 300. The leading portion 2904 can be heat set to deflect, have one or more shaping members (e.g., tension members, a nitinol tube or ribbon) coupled thereto, and/or the like. The leading portion 2904 can correspondingly deflect the inner catheter assembly 310 positioned therein and an aortic repair device secured thereto.

FIG. 29B is a side view of the delivery system 300 of FIG. 29A positioned at least partially within an aorta and configured to deploy an aortic repair device in accordance with embodiments of the present technology. In the illustrated embodiment, the delivery system 300 is inserted through the brachiocephalic artery such that the leading portion 2904 of the outer catheter 302 is positioned within the ascending aorta (e.g., proximate the sinotubular junction). In some aspects of the present technology, the shaped leading portion 2904 can help position the leading portion 2904 generally toward a center of the aorta and square the leading portion 2904 within the aorta. More specifically, the shaped leading portion 2904 can allow the delivery system 300 to traverse the curvature of the ascending aorta to center the delivery system 300 therein.

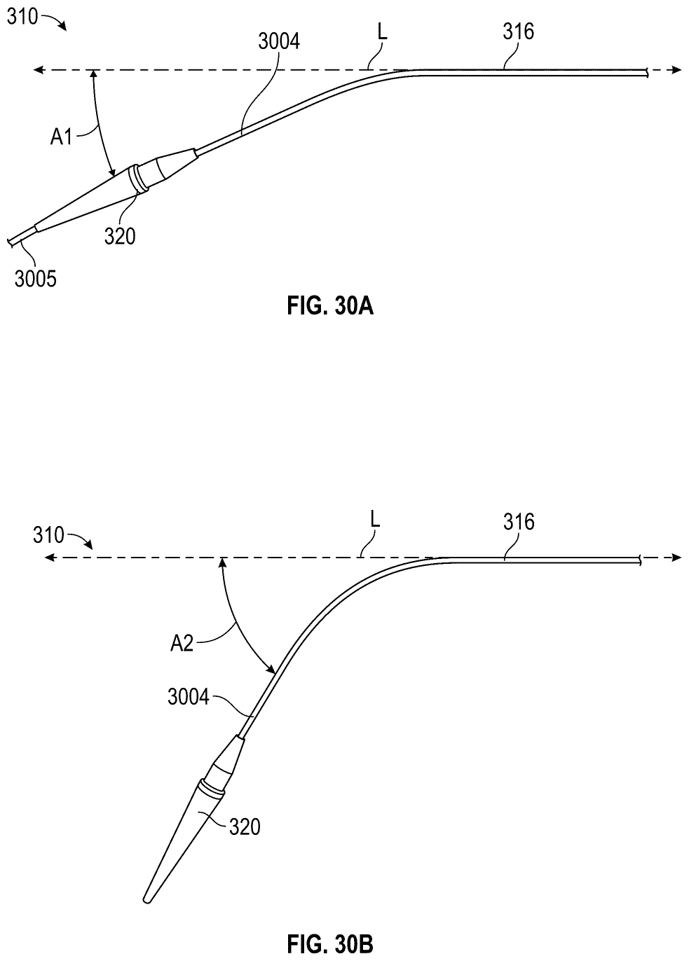

FIGS. 30A and 30B are perspective side views of a distal portion of the inner catheter assembly 310 of the delivery system 300 of FIGS. 3A-3G in a first position and a second position, respectively, in accordance with embodiments of the present technology. In the illustrated embodiment, the inner catheter 316 includes a leading portion 3004 shaped to deflect/extend away from a longitudinal axis L of the inner catheter 316. The leading portion 3004 can be heat set to deflect, have one or more shaping members (e.g., tension members, a nitinol tube or ribbon) coupled thereto, and/or the like.

Referring to FIG. 30A, the inner catheter 316 and the tip member 320 can slidably receive a guidewire 3005. The guidewire 3005 can have a flexibility/stiffness selected such that, in the first position, the leading portion 3004 deflects by an angle A1 from the longitudinal axis L. In some embodiments, the angle A1 is between about 0°-40°, between about 20°-30°, about 25°, and/or the like. Referring to FIG. 30B, the guidewire 3005 (FIG. 30A) can be retracted at least partially through the leading portion 3004 of the inner catheter 316 in the second position to permit the leading portion 3004 to deflect to a greater angle A2. The angle A2 can be between about 30°-70°, between about 40°-60°, about 50°, and/or the like. In some embodiments, the guidewire 3005 can include a flexible portion (e.g., a floppy portion) that can be retracted into the leading portion 3004 of the inner catheter 316 to allow the leading portion 3004 to deflect to the second position. Accordingly, referring to FIGS. 30A and 30B, the guidewire 3005 can be selectively retracted through the leading portion 3004 of the inner catheter 316 to control an amount of deflection of the leading portion 3004 to control the position of an aortic repair device coupled thereto. Such steerability can facilitate implantation of the aortic repair device in a centered and/or square position within an aorta, as described in detail above.

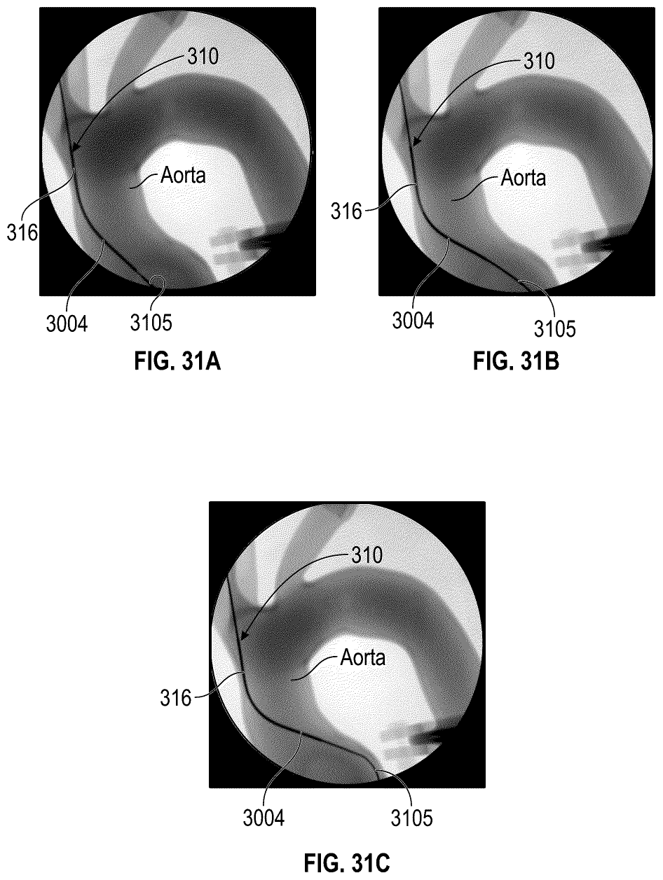

In some embodiments, the guidewire 3005 can have different sections (e.g., two or more sections) with varying degrees of flexibility/stiffness to provide for more or less deflection of the leading portion 3004 of the inner catheter 316 and/or to permit the leading portion 3004 to deflect without withdrawing the guidewire 3005 entirely into the inner catheter assembly 310. For example, FIGS. 31A-31C are side views (e.g., fluoroscopic images) of the inner catheter assembly 310 of FIGS. 30A and 30B inserted within an aorta of a patient over a guidewire 3105 and in a first position, a second position, and a third position, respectively, in accordance with embodiments of the present technology. The guidewire 3105 can have a distal first flexible section, a first stiff section extending proximally from the first flexible section, a second flexible section extending proximally from the first stiff section, and a second stiff section extending proximally from the second flexible section. In some embodiments, the first and second flexible sections can have a smaller diameter (e.g., formed by grinding the guidewire 3105) than the first and second stiff sections and/or can otherwise be made more flexible than the first and second stiff sections.

Referring to FIG. 31A, in the first position the guidewire 3105 can be inserted through the leading portion 3004 of the inner catheter 316 such that the first stiff section of the guidewire 3105 is positioned at least partially within the leading portion 3004. Accordingly, with additional reference to FIG. 30A, the leading portion 3004 can be deflected by the smaller angle A1.

Referring to FIG. 31B, in the second position the guidewire 3105 can be advanced through the leading portion 3004 of the inner catheter 316 such that a portion of the first stiff section and a portion of the second flexible section of the guidewire 3105 are positioned partially within the leading portion 3004. Accordingly, with additional reference to FIGS. 30A and 30B, the leading portion 3004 can be deflected by an intermediate angle larger than the angle A1 and smaller than the angle A2.

Referring to FIG. 31C, in the third position the guidewire 3105 can be further advanced through the leading portion 3004 of the inner catheter 316 such that the second flexible section of the guidewire 3105 is positioned at least partially within the leading portion 3004. Accordingly, with additional reference to FIG. 30B, the leading portion 3004 can be deflected by the larger angle A2.

In some embodiments, the guidewire 3105 can be advanced/retracted to position all or a portion of the first flexible section, the first stiff section, the second flexible section, and/or the second stiff section within the leading portion 3004 of the inner catheter 316 to control the angle of deflection of the leading portion 3004. In this manner, the inner catheter assembly 310 can be steered via the guidewire 3105 to, for example, center and/or square the inner catheter assembly 310 within the aorta to facilitate deployment of an aortic repair device coupled thereto, as described in detail above. In some aspects of the present technology, the multiple flexible and stiff sections of the guidewire 3105 permit such steering without requiring the guidewire to be retracted fully into/through the leading portion 3004.

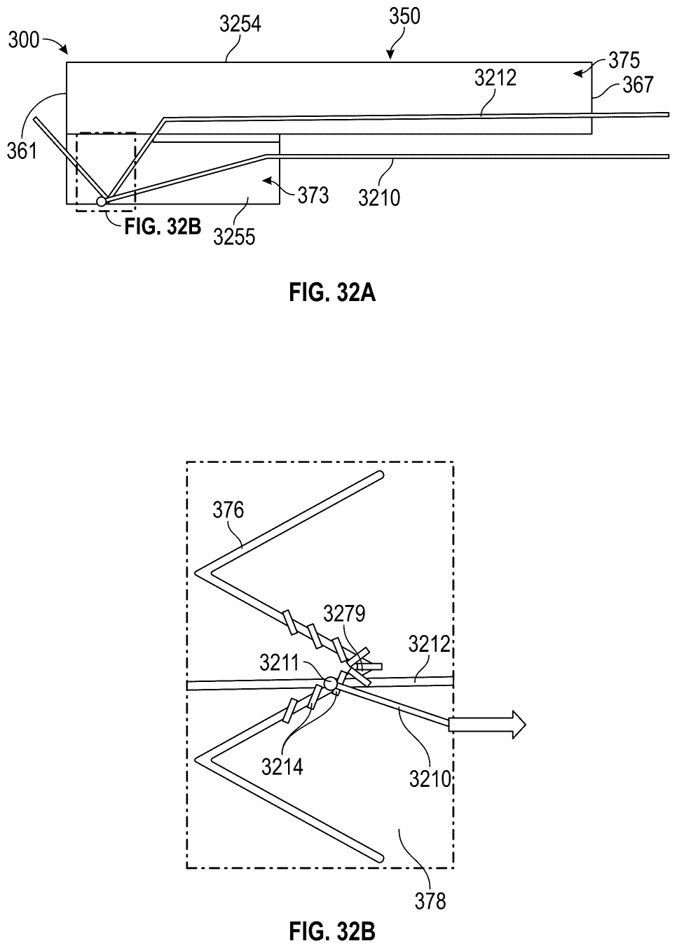

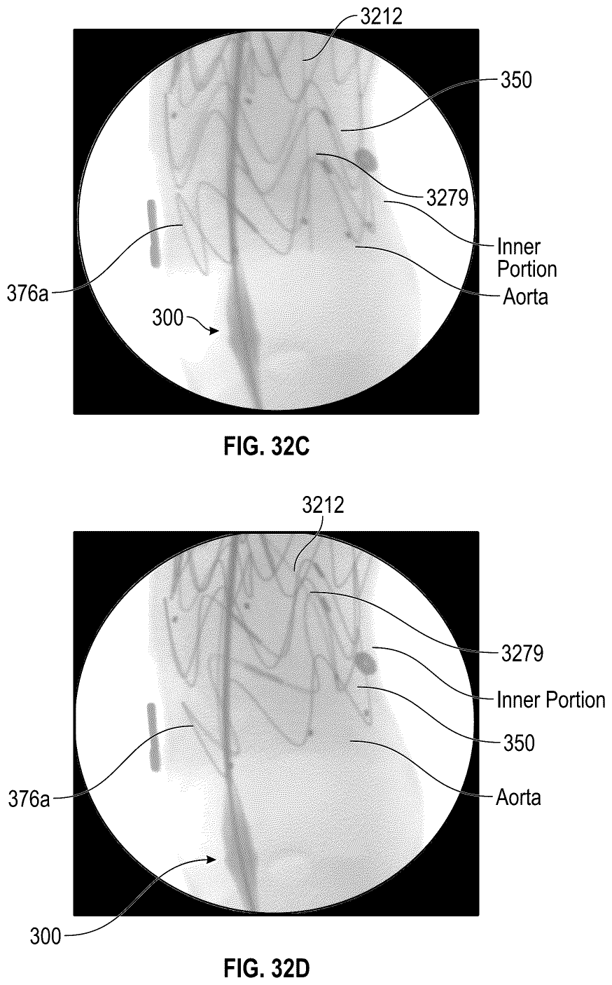

In some embodiments, a delivery system in accordance with embodiments of the present technology can include components for pulling one or more of the stents of an aortic repair device to improve the squareness of the aortic repair device within an aorta. FIG. 32A, for example, is a schematic side view of a distal portion of the delivery system 300 secured to the aortic repair device 350 of FIGS. 3A-3G in accordance with embodiments of the present technology. In the illustrated embodiment, the delivery system 300 further includes a tether 3210 releasably secured to the aortic repair device 350 via a release wire 3212. The tether 3210 can be a wire, fiber, suture, and/or the like. The tether 3210 can be pulled proximally to tension the aortic repair device 350, and the release wire 3212 can be pulled proximally to release/decouple the tether 3210 from the aortic repair device 3250. In the illustrated embodiment, the tether 3210 extends proximally through the primary lumen 373 and the release wire 3212 extends proximally through the secondary lumen 375. Referring to FIGS. 3A and 32A, the release wire 3212 and the tether 3210 can extend proximally through the outer catheter 302 and/or the pusher catheter 312. For example, referring to FIGS. 3D and 32A, the tether 3210 and the release wire 3212 can extend through different ones of the valves 326 for access by a user.

FIG. 32B is an enlarged side view of a portion of the delivery system 300 and the aortic repair device 350 of FIG. 32A in accordance with embodiments of the present technology. In the illustrated embodiment, the tether 3210 is attached to one of the stents 376 (e.g., the leading stent 376a) and, more particularly, can be inserted through one or more sutures 3214 that secure the stent 376 to the graft material 378. In some embodiments, the suture 3214 is positioned near an apex 3279 of the stent 376, such as a trailing apex. In other embodiments, the tether 3210 can be secured to a different portion of the stent 376 and/or can be routed through one or more separate stitches, loops, and/or sutures proximate the stent 376 that do not secure the stent 376 to the graft material 378. To secure the tether 3210 to the aortic repair device 350, the knot 3211 can be positioned distal to the stent 376, and the release wire 3212 can extend through the knot 3211 to hold the knot 3211. In this configuration, the suture 3214 inhibits the knot 3211 from sliding proximally there past. That is, the release wire 3212 secures the knot 3211 such that, when the tether 3210 is pulled proximally, the suture 3241 contacts the knot 3211. In some embodiments, the release wire 3212 can also be used/routed to release the leading end portion 361 and/or the trailing end portion 367 from the leading and/or trailing tip capture mechanisms 342, 340 (FIGS. 3B and 3C). In other embodiments, the release wire 3212 is separate from the release wires used for the leading and/or trailing tip capture mechanisms 342, 340.

Referring to FIGS. 32A and 32B, in the illustrated embodiment the apex 3279 is positioned at/adjacent to a second side portion 3255 of the aortic repair device 3250 that is configured to conform to an inner portion (e.g., an inner curved portion) of an interior wall of an aorta having a lesser curvature (e.g., a smaller radius of curvature, shorter length). The second side portion 3255 is opposite a first side portion 3254 configured to conform to an outer portion (e.g., an outer curved portion) of the interior wall of the aorta having a greater curvature (e.g., greater radius of curvature, longer length). Accordingly, withdrawing the tether 3210 proximally can pull/retract the apex 3279 and the stent 376 proximally to adjust a position (e.g., tilt) of the aortic repair device 350 within an aorta and, in particular, can pull the second side portion 3255 more than the first side portion 3254. In some aspects of the present technology, the release wire 3212 extends generally longitudinally from the apex 3279 through the primary lumen 373 such that retraction of the release wire 3212 pulls the apex 3279 generally longitudinally rather than radially. Withdrawing the release wire 3212 proximally through the knot 3211 can allow the knot 3211 to slide proximally past the suture 3214 to thereby release the aortic repair device 350 from the tether 3210. The knot 3211 can be small enough after retraction of the release wire 3212 to slide past the suture 3214 without catching on the suture 3214. In some embodiments, the tether 3210 is coupled to multiple apices of the stent 376, or multiple ones of the stents 3276 of aortic repair device 3250.

FIGS. 32C and 32D are side views (e.g., fluoroscopic images) of a first stage and a second stage, respectively, of a procedure to implant the aortic repair device 350 within an aorta of a patient using the delivery system 300 of FIGS. 32A and 32B in accordance with embodiments of the present technology. Referring to FIG. 32C, in the first stage the aortic repair device 350 is shown deployed from the outer catheter 302 (FIG. 3A) within the aorta and after releasing the leading stent 376a from the leading tip capture mechanism 342 (FIG. 3A). Referring to FIGS. 32A, 32B, and 32D, in the second stage the tether 3210 has been retracted proximally to retract the apex 3279 of the leading stent 376a. Retraction of the apex 3279 can also retract a portion of other apices of the leading stent 376a. In some embodiments, retracting the apex 3279 can (i) square the aortic repair device 350 within the aorta, (ii) move the leading stent 376a away from a left coronary artery branching from the aorta to inhibit or even prevent the aortic repair device 350 from blocking/covering the left coronary artery, and/or (iii) help the aortic repair device 350 conform to the inner portion of the interior wall of the aorta having the lesser curvature. After retracting the apex 3279, the release wire 3212 can be pulled proximally in a third stage to decouple the tether 3210 from the aortic repair device 3250.

Referring to FIGS. 3C and 32A-32D, in some embodiments the tether 3210 can be pulled to retract the apex 3279 and adjust the position/orientation of the aortic repair device 3250 before the leading end portion 361 is released from the leading tip capture mechanism 342. For example, the leading end portion 361 (e.g., the leading stent 376a) can be loosely secured to the leading tip capture mechanism 342, as described in detail above with reference 13B, such that the apex 379 can be retracted while remaining coupled to the leading tip capture mechanism 342.

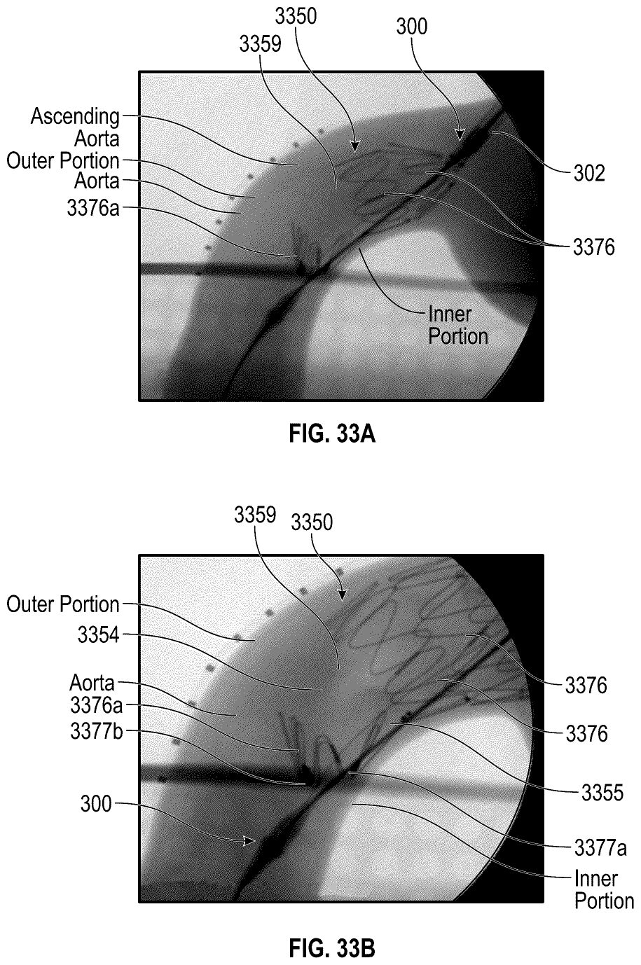

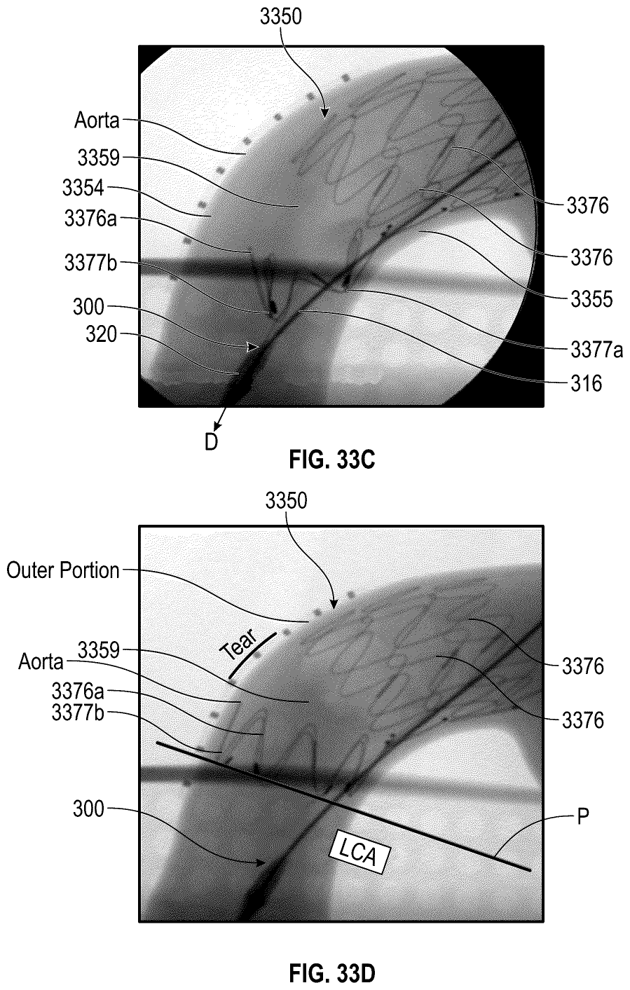

FIGS. 33A-33D are side views (e.g., fluoroscopic images) of different stages of a procedure to implant an aortic repair device 3350 within an aorta of a patient using the delivery system 300 of FIGS. 3A-3G in accordance with embodiments of the present technology. In the illustrated embodiment, the aortic repair device 3350 includes multiple stents 3376 (including an individually identified leading stent 3376a) secured to a graft material (obscured in FIGS. 33A-33D). The leading stent 3376a is configured to be releasably secured to the leading tip capture mechanism 342 (FIGS. 3B and 3C).

Referring to FIG. 33A, in a first stage the aortic repair device 3350 can be partially deployed from the outer catheter 302 at a target position within the aorta. For example, three of the stents 3376 are shown deployed from the outer catheter 302 within the ascending aorta. In the illustrated embodiment, the leading stent 3376a is cinched and secured to the leading tip capture mechanism 342 (FIGS. 3B and 3C). In some embodiments, the target position is an intended position for the leading stent 3376a to contact an inner portion (e.g., an inner curved portion) of an interior wall of the aorta having a lesser curvature (e.g., a smaller radius of curvature, shorter length). The inner portion of the aorta is opposite an outer portion (e.g., an outer curved portion) of the interior wall of the aorta having a greater curvature (e.g., greater radius of curvature, longer length).

Referring to FIG. 33B, in a second stage the aortic repair device 3350 can be further (e.g., fully) deployed from the outer catheter 302 (FIG. 33A) to allow the aortic repair device 3350 to expand. Further, the leading tip capture mechanism 342 (FIGS. 3A and 3C) can be actuated in a first release stage to release one or more first apices 3377a (e.g., leading apices) of the leading stent 3376a while still capturing and securing one or more second apices 3377b (e.g., leading apices) of the leading stent 3376a. In some embodiments, (i) the first apices 3377a are positioned adjacent to a first side portion 3355 of the aortic repair device 3350 that is configured to conform to the inner portion of the aorta, and (ii) the second apices 3377b are positioned adjacent to a second side portion 3354 of the aortic repair device 3350 opposite the first side portion 3355 that is configured to conform to the outer portion.

Referring to FIG. 33C, in a third stage the inner catheter 316 and the tip member 320 can be advanced distally within the aorta in the direction of arrow D. As described in detail above with reference to FIGS. 3A, 3C, and 7A-23, the leading tip capture mechanism 342 can secure the second apices 3377b of the leading stent 3376a to the tip member 320 and/or the inner catheter 316. Accordingly, advancement of (e.g., forward traction on) the inner catheter 316 and the tip member 320 drives the second apices 3377b distally within the aorta in the direction of the arrow D relative to the first apices 3377a to, for example, tilt and square the leading stent 3376a within the aorta. In some aspects of the present technology, such movement of the leading stent 3376a acts to elongate the second side portion 3354 of the aortic repair device 3350 relative to the first side portion 3355.

Referring to FIG. 33D, in a fourth stage the leading tip capture mechanism 342 (FIGS. 3A and 3C) can be actuated in a second release stage to release the second apices 3377b and allow the leading stent 3376a to fully expand within the aorta. In the illustrated embodiment, the leading stent 3376a and a corresponding leading end portion of the aortic repair device 3350 extend along a plane P after deployment that can be squarely positioned within the aorta (e.g., aligned with a plane of a sinotubular junction of the aorta) and that can be positioned above a left coronary artery LCA branching from the aorta. Accordingly, in some aspects of the present technology, advancing the second apices 3377b relative to the first apices 3377a during deployment (FIGS. 33B and 33C) can improve the squareness of the aortic repair device 3350 after implantation while also inhibiting or even preventing the aortic repair device 3350 from covering/blocking the left coronary artery LCA. Additionally, the second apices 3377b can be positioned farther proximally within the aorta (e.g., closer to the aortic valve, the sinotubular junction, etc.) such that the aortic repair device 3350 covers more of the outer portion of the aorta to provide a longer treatment/scaling region. Such a longer treatment region can allow the aortic repair device 3350 to cover and treat tears (e.g., dissections) in the outer portion that may be positioned farther proximally within the aorta.

Referring to FIGS. 33A-33D, in the illustrated embodiment the aortic repair device 3350 includes a region 3359 at which one of the stents 3376 is omitted and/or at which there is only graft material. The region 3359 can be more flexible than other regions of the aortic repair device 3350 including the stents 3376 and, in some aspects of the present technology, can help the second side portion 3354 conform to the outer portion of the aorta during deployment.

In some embodiments, the delivery system 300 can be used to achieve a similar or identical deployed state in which the leading stent 3276a is tilted via retraction rather than advancement of the inner catheter 316 and the tip member 320. For example, referring to FIG. 33A, in the first stage the aortic repair device 3350 can be deployed from the outer catheter 302 at a target position for the leading stent 3376a to contact the outer portion of the aorta. Then, referring to FIG. 33B, in the second stage the leading tip capture mechanism 342 (FIGS. 3A and 3C) can be actuated in a first release stage to release the second apices 3377b while still capturing and securing the first apices 3377a. Next, in the third stage shown in FIG. 33C, the inner catheter 316 and the tip member 320 can be retracted proximally within the aorta to pull the first apices 3377a proximally within the aorta relative to the second apices 3377b to tilt the leading stent 3376a and shorten the first side portion 3355 of the aortic repair device 3350 relative to the second side portion 3354. Finally, referring to FIG. 33D, the leading tip capture mechanism 342 (FIGS. 3A and 3C) can be actuated in a second release stage to release the first apices 3377a and allow the leading stent 3376a to fully expand within the aorta.

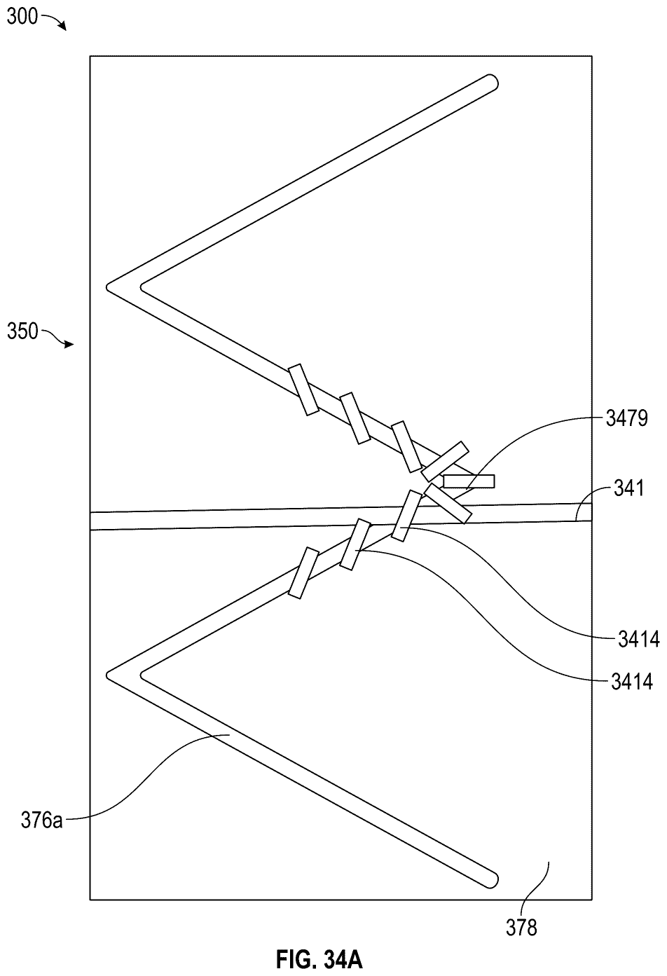

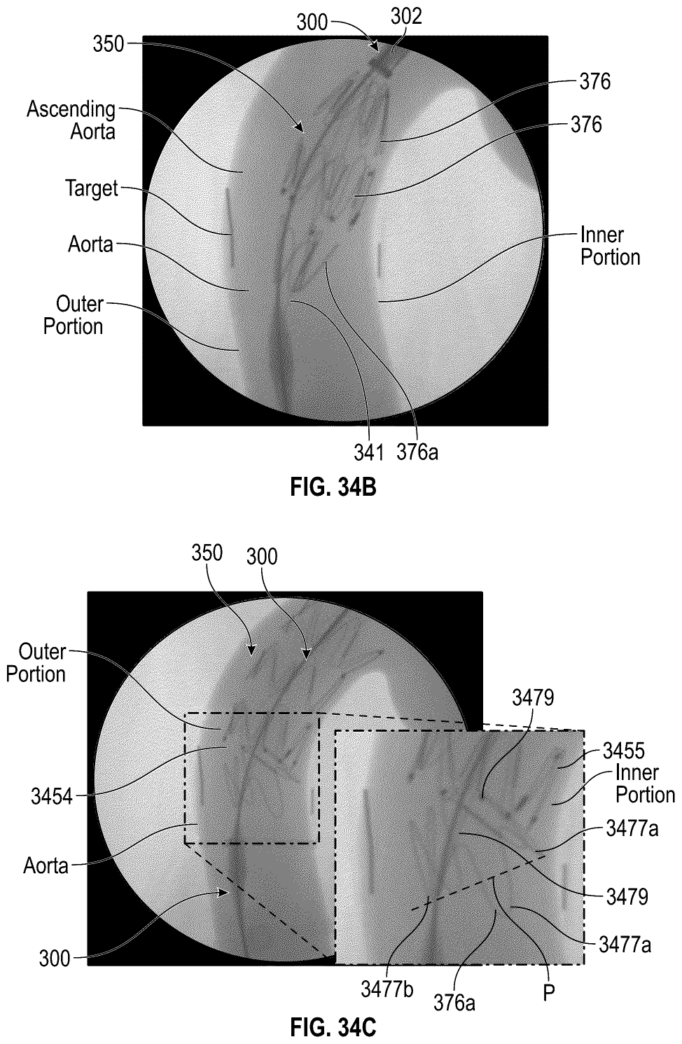

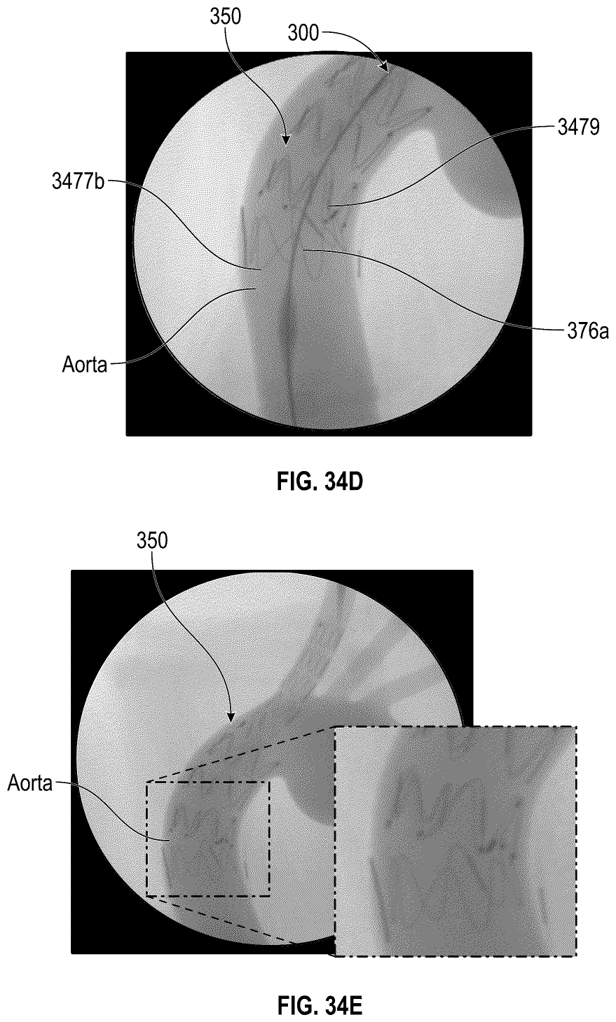

In some embodiments, the delivery system 300 can be used to achieve a similar or identical deployed state in which a leading stent of an aortic repair device is tilted passively via the routing of a release wire rather than via active advancement/retraction of the inner catheter 316 and the tip member 320. FIG. 34A, for example, is an enlarged side view of a portion of the delivery system 300 and the aortic repair device 350 of FIGS. 3A-3G in accordance with embodiments of the present technology. In the illustrated embodiment, the leading stent 376a is secured to the graft material 378 via one or more sutures 3414. One of the release wires 341 (e.g., a release wire for actuating the leading tip capture mechanism 342 and/or the trailing tip capture mechanism 340 shown in FIGS. 3B and 3C) can extend through one or more of the sutures 3414 proximate to, for example, an apex 3479 of the leading stent 376a, such as a trailing apex. In some embodiments, the apex 3479 is positioned at/adjacent to a side portion of the aortic repair device 350 that is configured to conform to an inner portion (e.g., an inner curved portion) of an interior wall of an aorta having a lesser curvature (e.g., a smaller radius of curvature, shorter length) that an outer portion. In other embodiments, the release wire 341 can be secured to a different portion of the leading stent 376a and/or can be routed through one or more separate stitches, loops, and/or sutures proximate the leading stent 376a that do not secure the leading stent 376a to the graft material 378. Referring to FIGS. 3C and 34A, the release wire 341 can extend at least partially through an interior of the aortic repair device 350. For example, the release wire can extend through the primary lumen 373 and touch or “kiss” an inner wall of the body 362 within the primary lumen 373 by extending under the suture 3414.

FIGS. 34B-34E are side views (e.g., fluoroscopic images) of different stages of a procedure to implant the aortic repair device 350 within an aorta of a patient using the delivery system 300 of FIG. 34A in accordance with embodiments of the present technology. Referring to FIG. 34B, in a first stage the aortic repair device 350 can be partially deployed from the outer catheter 302 at a target position within the aorta. For example, three of the stents 376 are shown deployed from the outer catheter 302 within the ascending aorta. In the illustrated embodiment, the leading stent 376a is cinched and secured to the leading tip capture mechanism 342 (FIGS. 3B and 3C) via the release wire 341. In some embodiments, the target position is at a target position for the leading stent 376a to contact an outer portion (e.g., an outer curved portion) of an interior wall of the aorta having a greater curvature (e.g., greater radius of curvature, longer length) than an inner portion having a greater curvature (e.g., greater radius of curvature, longer length). The outer portion can have a tear or dissection targeted for treatment with the aortic repair device 350.

Referring to FIG. 34C, in a second stage the aortic repair device 350 can be further (e.g., fully) deployed from the outer catheter 302 (FIG. 34B) to allow the aortic repair device 3350 to expand. Further, the leading tip capture mechanism 342 (FIGS. 3B and 3C) can be actuated in a first release stage to release one or more first apices 3377a (e.g., leading apices) of the leading stent 376a while still capturing and securing one or more second apices 3477b (e.g., leading apices) of the leading stent 376a. In some embodiments, (i) the first apices 3477a are positioned adjacent to a first side portion 3455 of the aortic repair device 350 that is configured to conform to the inner portion of the aorta, and (ii) the second apices 3377b are positioned adjacent to a second side portion 3454 of the aortic repair device 350 opposite the first side portion 3455 that is configured to conform to the outer portion. When the first apices 3477a are released and expanded, the release wire 341 can pull the apex 3479 of the leading stent 376a radially inward and/or distally within the aorta to, for example, tilt and square the leading stent 376a within the aorta. For example, in the illustrated embodiment the leading stent 376a and a corresponding leading end portion of the aortic repair device 350 extend along a plane P that can be squarely positioned within the aorta (e.g., aligned with a plane of a sinotubular junction of the aorta) and that can be positioned above a left coronary artery branching from the aorta. In some embodiments, the release wire 341 inhibits the first side portion 3455 from fully expanding.

Referring to FIG. 34D, in a third stage the leading tip capture mechanism 342 (FIGS. 3A and 3C) can be actuated in a second release stage to release the second apices 3477b and allow the leading stent 376a to expand. In some embodiments, the apex 3479 of the leading stent 376a can remain coupled to the release wire (FIGS. 34A-34C) during release of the second apices 3477b. In other embodiments, the release wire 341 is retracted before releasing the second apices 3477b, or generally simultaneously with the second apices 3477b when, for example, the release wire 341 is configured to release the second apices 3477b from the leading tip capture mechanism 342.

Referring to FIG. 34E, in a fourth and final stage the delivery system 300 can be withdrawn from the patient to leave the aortic repair device 350 implanted within the aorta. Referring to FIGS. 34A-34E, in other embodiments the release wire 341 can be additionally or alternatively be coupled to another one of the stents 376 other than the leading stent 376a. In some aspects of the present technology, coupling the release wire to the leading stent 376a can induce a greater tilt in the leading stent 376a when the leading stent 376a is released from the leading tip capture mechanism 342 (FIGS. 3B and 3C). Moreover, the release wire 341 can be a dedicated wire that is not operably coupled to either of the leading or trailing tip capture mechanisms 342, 340 (FIGS. 3B and 3C). Further, the delivery system 300 can include multiple ones of the release wires 341 coupled to different apices of the same one of the stents 376, or different ones of the stents 376, to induce different amounts and/or orientations of tilt of the leading stent 376a.

### V. SELECTED EMBODIMENTS OF LOADING FUNNELS

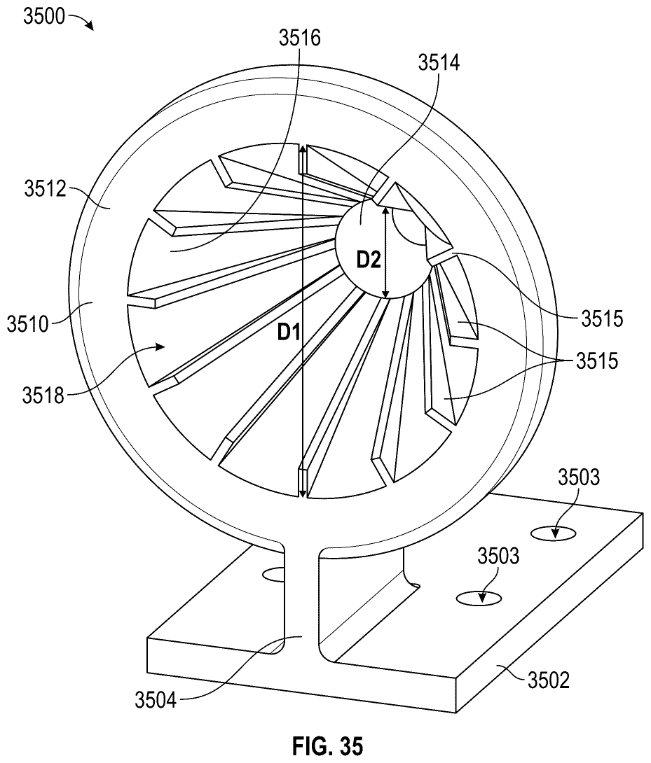

In some embodiments, a delivery system in accordance with embodiments of the present technology can include a funnel device for loading an aortic repair device into an outer catheter of the delivery system in a compressed position. FIG. 35, for example, is an isometric view of a funnel device 3500 in accordance with embodiments of the present technology. In the illustrated embodiment, the funnel device 3500 includes a base 3502, a stand 3504 extending from the base 3502, and a funnel member 3510 coupled to the stand 3504. The base 3502 can include one or more holes or apertures 3503 configured (e.g., shaped, sized, positioned) to receive corresponding fasteners (not shown) for fixedly securing the base 3502 to a surface.

In the illustrated embodiment, the funnel member 3510 includes a first side portion 3512, a second side portion 3514, and an inner surface 3516 extending between the first and second side portions 3512, 3514 and defining a lumen 3518. The lumen 3518 has a first diameter D1 at the first side portion 3512 and a second diameter D2 at the second side portion 3514 less than the first diameter D1. In some embodiments, the first diameter D1 generally corresponds to the diameter of an aortic repair device in an expanded and unconstrained state, and the second diameter D2 corresponds to the diameter of an outer catheter into which the aortic repair device is to be compressed, packed, and/or loaded into. The outer catheter can be coupled to the second side portion 3514 such that a lumen of the outer catheter is contiguous with the lumen 3518 of the funnel member 3510. In the illustrated embodiment, the funnel member 3510 further includes a plurality of ribs 3515 spaced circumferentially about the inner surface 3516 and extending at least partially between the first side portion 3512 and the second side portion 3514.

In operation, a user can insert the aortic repair device through the lumen 3518 from the first side portion 3512 toward the second side portion 3514 to collapse the aortic repair device into the outer catheter at the second side portion 3514. More specifically, the inner surface 3516 and the ribs 3515 can contact the aortic repair device to compress the aortic repair device from the expanded state to the compressed state. In some aspects of the present technology, the ribs 3515 can pleat loose graft material of the aortic repair device to move the graft material toward an interior of the aortic repair device and the outer catheter to improve packing (e.g., to more compactly compress and pack the aortic repair device within the outer catheter). In some embodiments the funnel device 3500 can include a source of air (e.g., cold air) and can route the air into the lumen 3518 via, for example, apertures or slots in the ribs 3515 and/or the inner surface 3516 to further pleat the graft material of the aortic repair device in a preferred direction (e.g., radially inward).

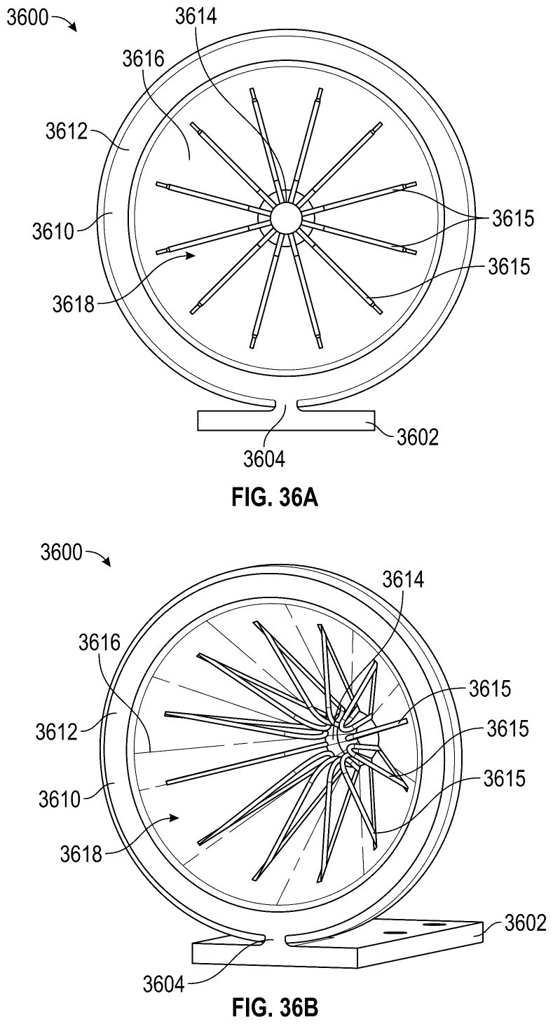

FIGS. 36A and 36B are a front view and an isometric view, respectively, of a funnel device 3600 in accordance with additional embodiments of the present technology. Referring to FIGS. 36A and 26B, the funnel device 3600 can include some features that are at least generally similar in structure and function, or identical in structure and function, to the corresponding features of the funnel device 3500 described in detail above with reference to FIG. 35 and can operate in a generally similar or identical manner to the funnel device 3500. For example, in some embodiments the funnel device 3500 includes a base 3602, a stand 3604 extending from the base 3602, and a funnel member 3610 coupled to the stand 3604. The funnel member 3610 can include a first side portion 3612, a second side portion 3614, and an inner surface 3616 extending between the first and second side portions 3612, 3614 and defining a lumen 3618 that decreases in diameter from the first side portion 3612 toward the second side portion 3614.

In the illustrated embodiment, the funnel member 3610 further includes a plurality of springs 3615 spaced circumferentially about the inner surface 3616 and extending at least partially between the first side portion 3612 and the second side portion 3614. In operation, a user can insert an aortic repair device through the lumen 3618 from the first side portion 3612 toward the second side portion 3614 to collapse the aortic repair device into the outer catheter at the second side portion 3614. In some aspects of the present technology, the spring members 3615 can pleat loose graft material of the aortic repair device to move the graft material toward an interior of the aortic repair device and the outer catheter to improve packing (e.g., to more compactly compress and pack the aortic repair device within the outer catheter). In some embodiments the funnel device 3600 can include a source of air (e.g., cold air) and can route the air into the lumen 3618 via, for example, apertures or slots in the spring members 3615 and/or the inner surface 3616 (e.g., through slots positioned below the spring members 3615) to further pleat the graft material of the aortic repair device in a preferred direction (e.g., radially inward).

### VI. SELECTED EMBODIMENTS OF DELIVERY SYSTEMS HAVING DEVICES FOR RADIALLY CONSTRAINING AN AORTIC REPAIR DEVICE DURING IMPLANTATION WITHIN AN AORTA

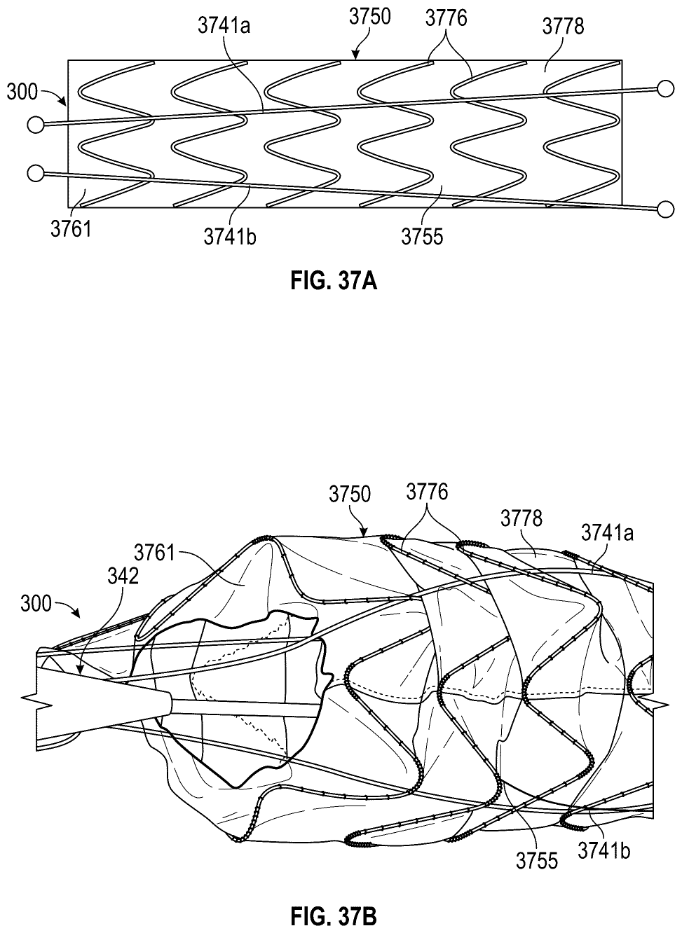

In some embodiments, a delivery system in accordance with embodiments of the present technology can include one or more devices for radially constraining an aortic repair device during implantation within an aorta and after deployment from a delivery catheter. FIGS. 37A-37C, for example, are a side view, a perspective side view, and another perspective side view, respectively, of a distal portion of the delivery system 300 of FIGS. 3A-3G secured to an aortic repair device 3750 in accordance with embodiments of the present technology. Referring to FIGS. 37A-37C, the aortic repair device 3750 is shown deployed and expanded from the outer catheter 302 (FIG. 3A) of the delivery system 300 and includes a plurality of stents 3776 coupled to a graft material 3778. The delivery system 300 includes one or more release wires 3741 (e.g., an individually identified first release wires 3741a and a second release wire 3741b) that can, for example, releasably secure a leading end portion 3761 of the aortic repair device 3750 a tip capture mechanism (e.g., the leading tip capture mechanism 342 shown in FIG. 37B).

The release wires 3741 can extend outside/over the graft material 3778 and one or more of the stents 3776 along, for example, a side portion 3755 of the aortic repair device 3750 that is configured to conform to an inner portion (e.g., an inner curved portion) of the interior wall of the aorta having a lesser curvature (e.g., a smaller radius of curvature, shorter length). Tension in the release wires 3741 can constrain a diameter of the aortic repair device 3750 when deployed from the outer catheter 302 (FIG. 3A) within an aorta. More specifically, the release wires 3741 can exert a radially inward force against the side portion 3755 of the aortic repair device 3750 that holds the side portion 3755 at least partially radially inward. A diameter of the release wires 3741 and/or tension within the release wires 3741 can be selected to provide a desired amount of radial compression. In some embodiments, the release wires 3741 are secure to the aortic repair device 3750 via suture loops or other fastening mechanisms. In some aspects of the present technology, constraining the expansion of the aortic repair device 3750 via the release wires 3741 can promote nesting/shingling of the stents 3776 along the side portion 3755 to improve the positioning (e.g., squareness, conformance to the inner portion of the interior wall of the aorta) of the aortic repair device 3750 within the aorta. In the illustrated embodiment, the release wires 3741 are angled relative to a longitudinal axis of the aortic repair device 3750 such that a separation distance between the release wires 3741 is smallest at the leading end portion 3761 and increases at least partially in a direction toward a trailing end portion of the aortic repair device 3750. Such angling can increase the radial constraint of the leading end portion 3761 to, for example, improve the squareness of the leading end portion 3761 within the aorta while promoting nesting/shingling in the direction toward the trailing end portion.

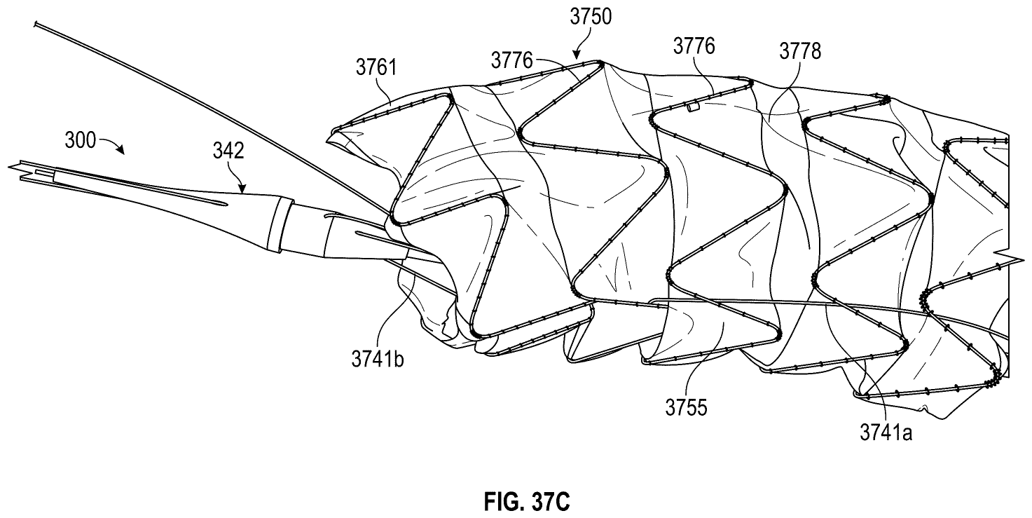

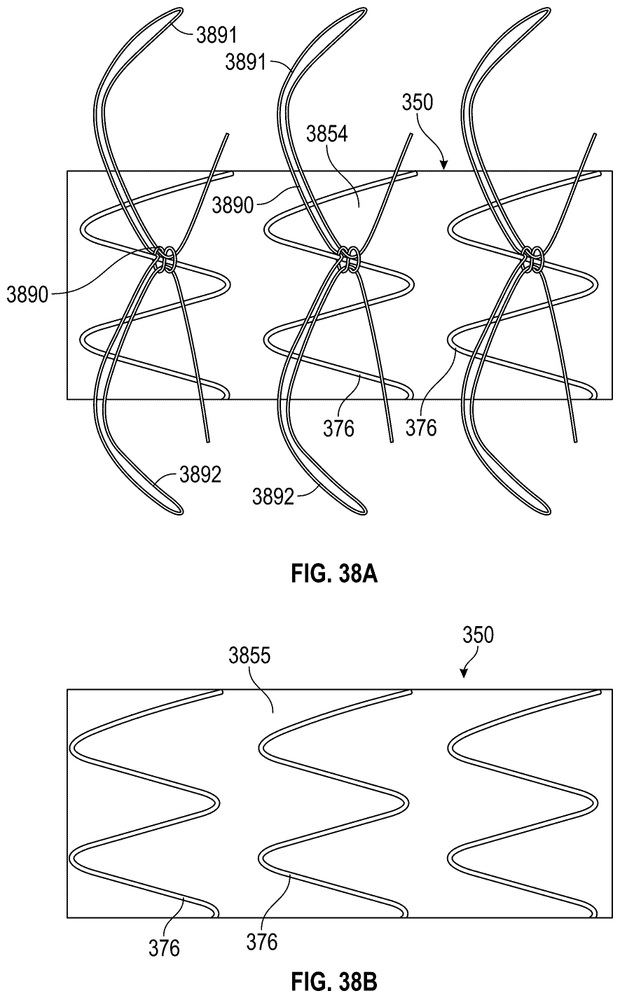

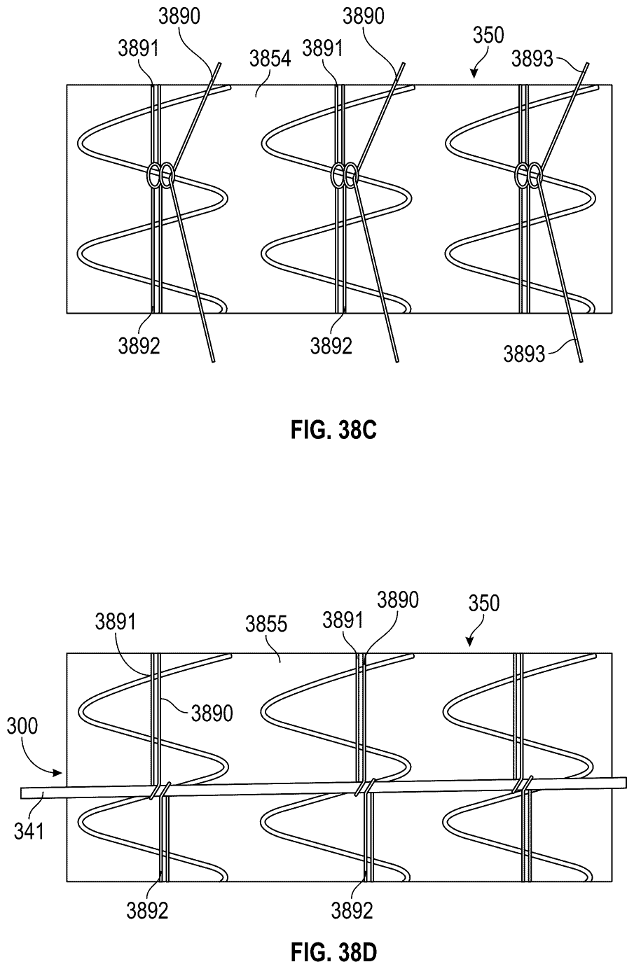

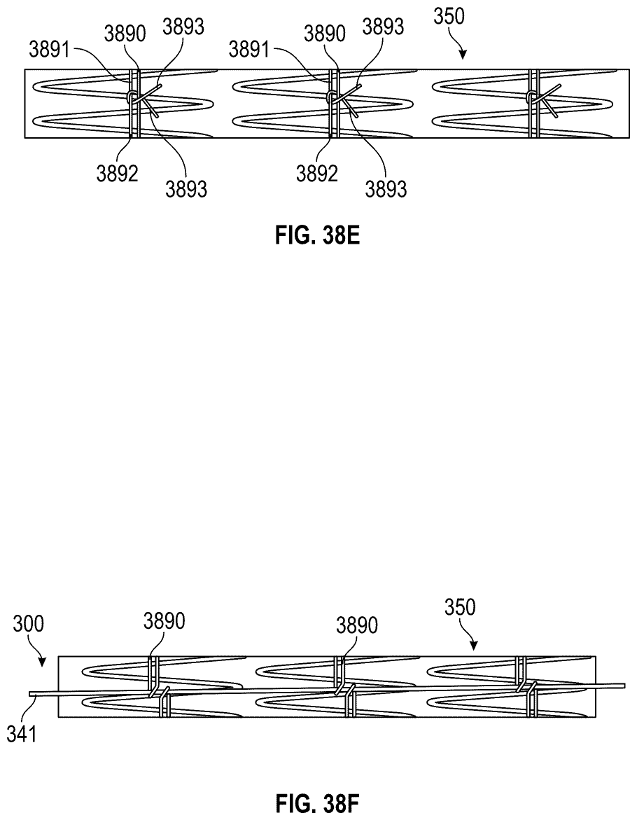

FIGS. 38A, 38C, and 38E are side views illustrating different stages of securing a first side portion 3854 of the aortic repair device 350 of FIG. 3C to the delivery system 300 of FIGS. 3A-3G in accordance with embodiments of the present technology. FIGS. 38B, 38D, and 38F are side views illustrating corresponding stages of securing a second side portion 3855 of the aortic repair device 350 of FIG. 3C to the delivery system 300 of FIGS. 3A-3G in accordance with embodiments of the present technology.

Referring to FIG. 38A, the aortic repair device 350 can include one or more constraining fibers 3890 (e.g., threads, metal wireless, sutures) secured to, for example, one or more of the stents 376 along the first side portion 3854. The constraining fibers 3890 can be secured to corresponding ones of the stents 376 via suturing, knotting, and/or the like to the stents 376. In the illustrated embodiment, the constraining fibers 3890 are each formed to have a first loop 3891 and a second loop 3892. Referring to FIG. 38B, at this stage, the second side portion 3855 remains unsecured/unconnected to the constraining fibers 3890.

Referring to FIGS. 38C and 38D, the first and second loops 3891, 3892 of the constraining fibers 3890 can be wrapped from the first side portion 3854 around the aortic repair device 3850 to the second side portion 3855, and one of the release wires 341 (FIG. 38D) can be inserted through the first and second loops 3891, 3892 of individual ones of the constraining fibers 3890 to secure the constraining fibers 3890 around the aortic repair device 3850. The release wire 341 can be integrated to include other wire functions (e.g., tip capture release), or can be a separate wire dedicated to hold, engage, and subsequently release the constraining fibers 3890. Referring to FIGS. 38E and 38F, the constraining fibers 3890 can then be cinched (e.g., tensioned) to radially compress/constrain the aortic repair device 3850. For example, free end portions 3893 (FIGS. 38C and 38E) of the constraining fibers 3890 can be pulled to reduce the size of the first and/or second loops 3891, 3892 and thereby compress the aortic repair device 3850. The constraining fibers 3890 (e.g., the free end portions 3893) can then be secured (e.g., knotted) secure the aortic repair device 3850 in the compressed state. During implantation of the aortic repair device 3850, the release wire 341 (FIG. 38F) can be retracted through the constraining fibers 3890 to decouple the first and second loops 3891, 3892 from one another to permit the aortic repair device 3850 to expand. In some embodiments, the constraining fibers 3890 remain behind with (e.g., fixed to) the aortic repair device 350 within the aorta after removal of the release wire 341.

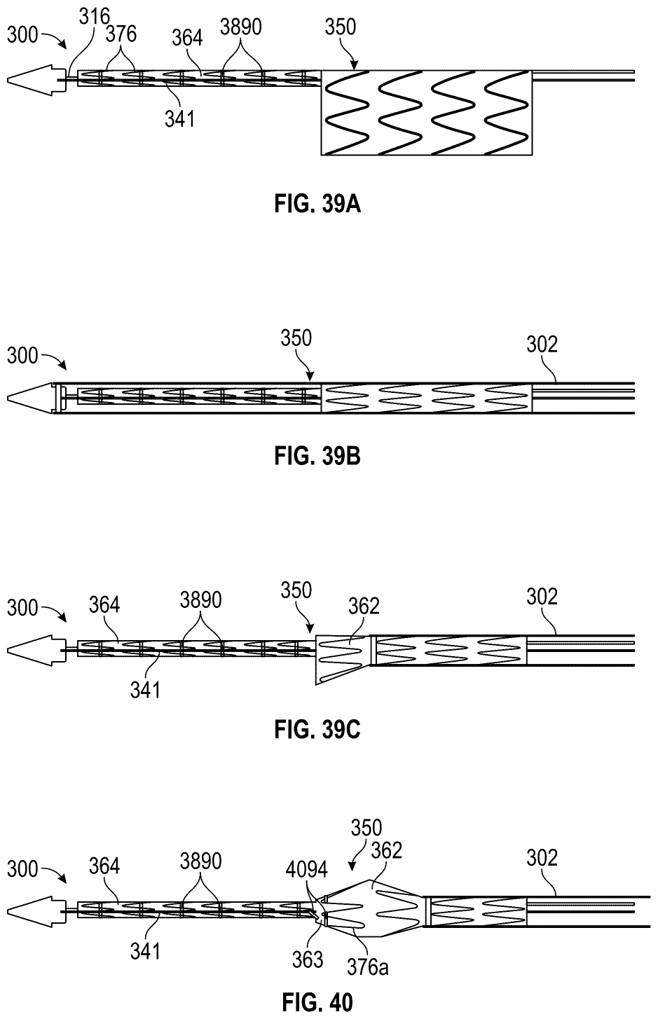

FIGS. 39A-39C illustrate different stages of deploying the aortic repair device 350 of FIGS. 38A-38D having the constraining fibers 3890 with the delivery system 300 of FIGS. 3A-3G in accordance with embodiments of the present technology. Referring to FIG. 39A, the constraining fibers 3890 are secured to corresponding ones of the stents 376 along the leg 364. The constraining fibers 3890 are tensioned to compress the leg 364 along the inner catheter 316 and secured in the tensioned/compressed state to the release wire 341.

Referring to FIG. 39B, the aortic repair device 350 has been compressed into the outer catheter 302 of the delivery system 300. Referring to FIG. 39C, the outer catheter 302 has been retracted (e.g., proximally) to partially deploy the aortic repair device 350 from the delivery catheter 302. In the illustrated embodiment, the leg 364 is deployed from the outer catheter 302 and the body 362 is partially deployed from the outer catheter 302. The engagement of the constraining fibers 3890 with the release wire 341 maintains the leg 364 in the compressed state even after deployment from the outer catheter 302. In some aspects of the present technology, by maintaining the leg 364 in the compressed state, the leg 364 can be selectively expanded within the aorta and/or a branch vessel via retraction of the release wire 341 past a subset (e.g., one or more) of the constraining fibers 3890 and/or the leg 364 can be fully withdrawn back into the outer catheter 302 for repositioning. More generally, constraining the leg 364 with the constraining fibers 3890 can permit safe repositioning (e.g., rotational, longitudinal) of the aortic repair device 350 during deployment.

FIG. 40 is a side view of the aortic repair device 350 of FIGS. 38A-39C secured to the delivery system 300 of FIGS. 3A-3G in accordance with additional embodiments of the present technology. In the illustrated embodiment, the aortic repair device 350 further includes a tip capture loop 4094 secured to an end portion 363 (e.g., a trailing or leading end portion depending on the ultimate orientation of the aortic repair device 350 within the aorta) of the body 362 of the aortic repair device 350. The loop 4094 can extend around/through corresponding apices (e.g., leading apices) of an end stent 376a positioned along the body 362 as described in detail above. The loop 4094 can be secured to the release wire 341 or a separate (e.g., dedicated) release wire to cinch the end portion 363 of the body 362 prior to retraction of the release wire 341. Thus, retraction of the release wire 341 can act to free the constraining fibers 3890 to allow the leg 364 to expand and to free the loop 4094 to allow the body 362 to expand. In some aspects of the present technology, the tip capture loop 4094 can maintain the end portion 363 of the body 362 in an at least partially compressed state after deployment from the outer catheter 302 to facilitate recapture of the body 362 into the outer catheter 302 and/or safe repositioning (e.g., rotational, longitudinal) of the aortic repair device 350 within the aorta.

### VII. SELECTED EMBODIMENTS OF FORWARD DRIVE MECHANISMS FOR DELIVERY SYSTEMS FOR IMPLANTING AORTIC REPAIR DEVICES

In some embodiments, a delivery system in accordance with embodiments of the present technology can include a forward drive mechanism to assist with positioning an aortic repair device within a target vessel of a patient. As used herein, the term “forward drive” refers to the ability of a delivery system to push or pull at least a portion of an implantable device in a distal or forward direction (e.g., away from the delivery system handle) relative to other system components (e.g., such as the outer catheter) at various stages of deployment.

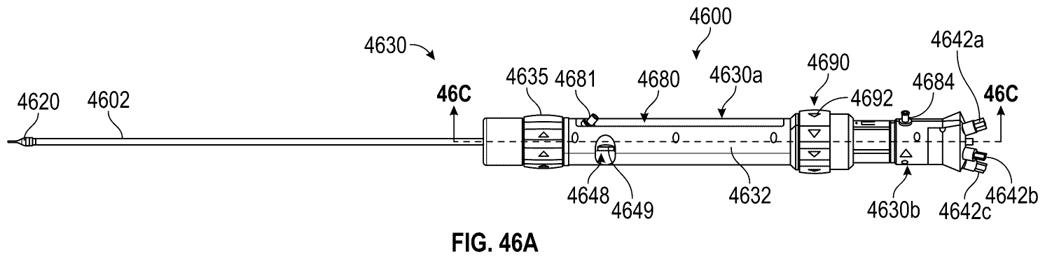

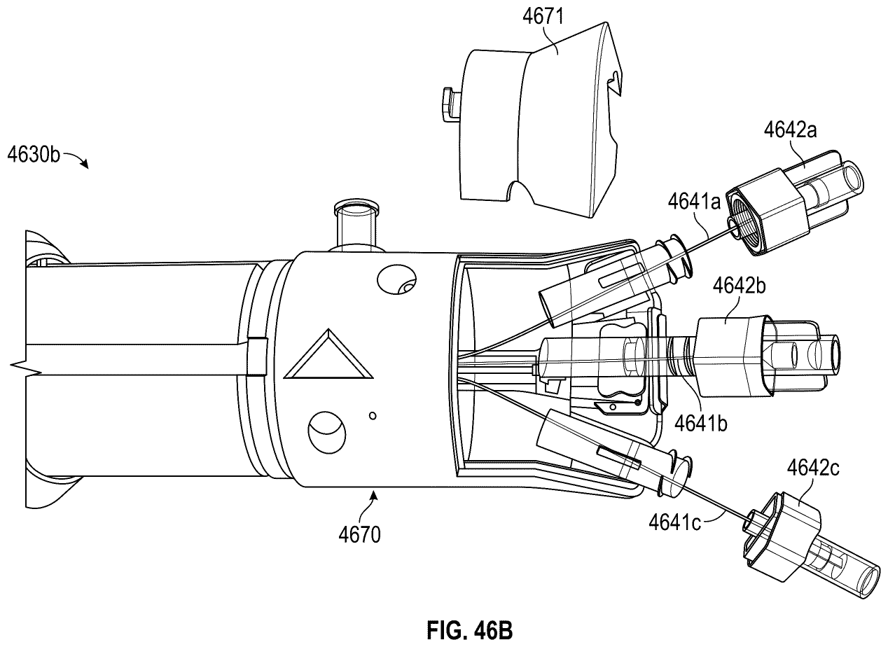

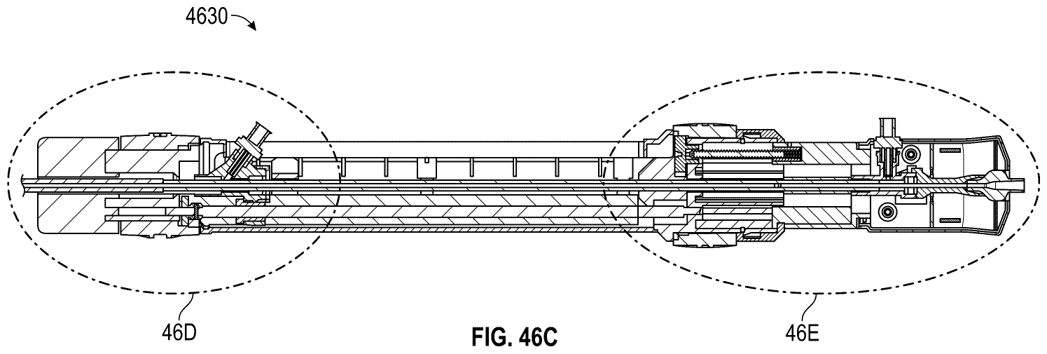

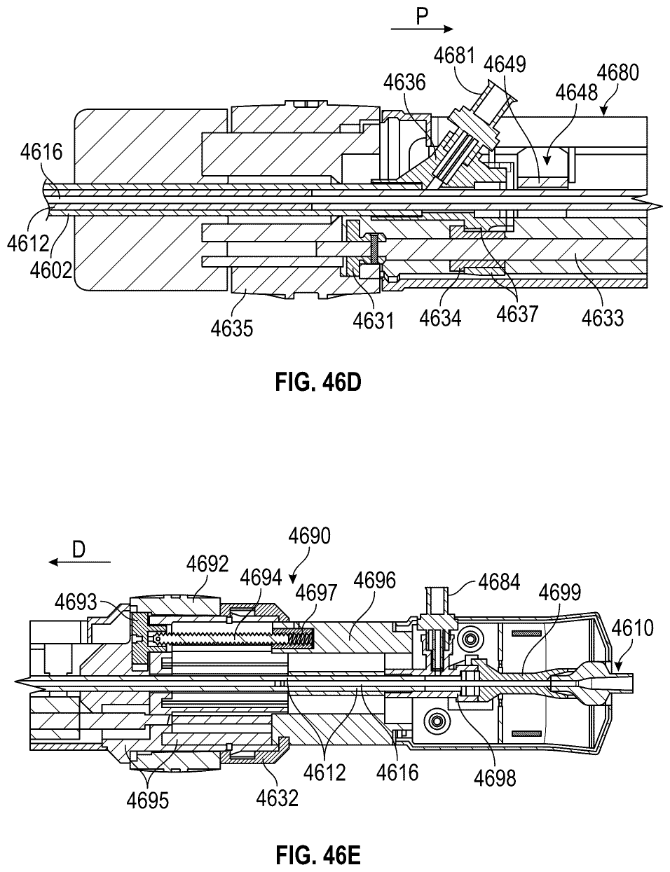

FIGS. 46A-46E illustrate a delivery system 4600 having a forward drive mechanism configured in accordance with embodiments of the present technology. More specifically, FIG. 46A is a side view of the delivery system 4600, FIG. 46B is an enlarged view of a proximal portion of a handle 4630 of the delivery system 4600, FIG. 46C is a cross-sectional view of the handle 4630 of the delivery system 4600 taken along the line indicated in FIG. 46A, and FIGS. 46D and 46E are enlarged views of portions of the cross-sectional view of FIG. 46C. The delivery system 4600 can include certain structures and functions similar to or the same as any of the other delivery systems described throughout this Detailed Description, except where the context clearly indicates otherwise.

As shown in FIG. 46A, the delivery system 4600 includes an outer catheter 4602, the handle 4630, and a tip member 4620. The outer catheter 4602 and the tip member 4620 can be generally similar to the outer catheter 302 and the tip member 320 of the delivery system 300 of FIGS. 3A-3G, and/or generally similar to corresponding features of the other delivery systems described throughout this Detailed Description. The delivery system 4600 can further include an inner catheter assembly (not visible in FIG. 46A) that can be generally similar to the inner catheter assembly 310 of the delivery system 300 of FIGS. 3A-3G, and/or generally similar to corresponding features of the other delivery systems described throughout this Detailed Description. Relative to previous embodiments, however, the handle 4630 includes a first handle portion 4630a and a second handle portion 4630b that is sized and shaped to be at least partially nested within (e.g., coaxial with) a housing 4632 of the first handle portion 4630a.

The second handle portion 4630b also includes a plurality of tip capture release knobs 4642. In particular, as best shown in FIG. 46B, the second handle portion 4630b can include a first release knob 4642a, a second release knob 4642b, and a third release knob 4642c. The first release knob 4642a can be coupled to a first release wire 4641a, the second release knob 4642b can be coupled to a second release wire 4641b, and the third release knob 4642c can be coupled to a third release wire 4641c. The release wires 4641a-c can extend to a proximal (e.g., leading) end of the delivery system (not visible in FIG. 46B) to releasably constrain one or more leading apices of a collapsed aortic repair device, as described above in Section III of this Detailed Description (e.g., as part of a tip capture mechanism). Indeed, the release wires 4641a-c and the delivery system 4600 can be used in connection with any of the tip capture mechanisms described herein. The release wires 4641a-c can extend from the handle 4630 to the leading end of the delivery system between the outer catheter 4602 (FIG. 46A) and the inner catheter assembly, or between subcomponents of the inner catheter assembly. In operation, a user can release the aortic repair device in specific stages by selectively pulling the release knobs 4642 in the proximal direction. To provide added visibility, the second handle portion 4630b can include a housing 4670 with a removeable panel 4671. A user can remove the panel 4671 to directly visualize the release wires 4641a-c. Removal of the panel 4671 also enables a user to directly grasp the release wires 4641a-c if needed to release the collapsed aortic repair device (e.g., as a fail-safe mechanism).

Returning to FIG. 46A, the handle 4630 further includes an actuator 4635 for retracting the outer catheter 4602 relative to the inner catheter assembly (not shown) and the tip member 4620 to unsheathe an aortic repair device carried by the delivery system 4600. For example, as shown in FIG. 46D, the actuator 4635 can be operably coupled to a leadscrew 4633 via a leadscrew connector 4631 (e.g., a geared connector) such that rotation of the actuator 4635 causes rotation of the leadscrew 4633. The leadscrew 4633 can itself be operably coupled to an outer catheter reverse driver 4636 via a leadscrew nut 4634 extending around the leadscrew 4633, such that rotational movement of the leadscrew 4633 induces translational movement of the outer catheter reverse driver 4636 in a proximal direction (labeled with an arrow P in FIG. 46D). In particular, the leadscrew nut 4634 can be connected to the leadscrew 4633 via a threaded connection, a tongue-and-groove connection, or other suitable connection such that rotation of the leadscrew 4633 causes translation of the leadscrew nut 4634 along the leadscrew 4633. The leadscrew nut 4634 can also be translationally fixed to a collar, annulus, or other feature 4637 extending from and integral with the outer catheter reverse driver 4636, such that translation of the leadscrew nut 4634 along the leadscrew 4633 drives a corresponding translation of the outer catheter reverse driver 4636 via engagement with the collar 4637. The outer catheter reverse driver 4636 can be fixedly coupled to the outer catheter 4602. Thus, actuation of the actuator 4635 causes the outer catheter reverse driver 4636 and the outer catheter 4602 to be retracted proximally in the direction P, which in operation can be used to expose an aortic repair device carried by the delivery system 4600 after the aortic repair device is positioned proximate at target deployment position within a vessel. In some embodiments, the actuator 4635 is designed to be rotatable in only a single direction that corresponds to retraction of the outer catheter 4602 in the proximal direction P (e.g., via a ratchet or other one-way mechanism). In other embodiments, the actuator 4635 is rotatable in two directions but limited to a predefined range of motion. For example, in the illustrated embodiment the outer catheter reverse driver 4636 cannot be advanced farther distally within the handle 4630 due to a physical engagement between the outer catheter reverse driver 4636 and an end of the slot 4680. This is expected to reduce the likelihood the outer catheter 4602 will inadvertently advance farther distally over the aortic repair device.

In some embodiments, the delivery system 4600 includes a secondary actuation mechanism to facilitate retraction of the outer catheter 4602 and provide redundant mechanisms for unsheathing the aortic repair mechanism. As shown in FIG. 46D, the first flush port 4681 can itself be fixedly coupled to the outer catheter reverse driver 4636, which as described above is fixedly coupled to the outer catheter 4602. Accordingly, instead of using the actuator 4635 to retract the outer catheter 4602, a user can simply pull the first flush port 4681 along the slot 4680 in the proximal direction P to pull the outer catheter reverse driver 4636, and thus the outer catheter 4602, in the proximal direction P. Without intending to be bound by theory, the first flush port 4681 can therefore operate as a manual fail-safe mechanism to retract the outer catheter 4602 in case the actuator 4635 does not operate to unsheathe the aortic repair device.

The delivery system 4600 can also include an actuator locking mechanism 4648 that prevents the unplanned movement of the outer catheter reverse driver 4636 and/or other handle actuator (e.g., caused by the force of device expansion). The actuator locking mechanism 4648 can include a bar, pin, or other feature 4649 (“the bar 4649”) that can transition between: (a) a first (e.g., locked) position in which it interferes with (e.g., blocks) the outer catheter reverse driver 4636 from moving in the proximal direction P, and thus prevents the outer catheter 4602 from retracting proximally, and (b) a second (e.g., unlocked) position in which it does not interfere with (e.g., does not block) the outer catheter reverse driver 4636 from moving the proximal direction P, and thus permits the outer catheter 4602 to retract proximally. In other embodiments, the actuator locking mechanism 4648 can be designed to interface with and prevent movement of other components associated with the actuator 4635, in addition to or in lieu of the outer catheter reverse driver 4636. For example, the bar 4649 can interface with the first flush port 4681, the leadscrew nut 4634, and/or the actuator 4635. Regardless of the particular embodiment, a user can toggle the actuator locking mechanism 4648 between the first and second positions by pushing or pulling the bar 4649 into or out of the housing 4632. The actuator locking mechanism 4648 is expected to reduce the risk of inadvertent retraction of the outer catheter 4602 (and thus inadvertent deployment of the aortic repair device) as the delivery system 4600 advances the outer catheter 4602 and the aortic repair device toward its target location and/or during partial expansion of the aortic repair device.

Returning to FIG. 46A, the delivery system 4600 further includes a forward drive mechanism 4690 including a forward drive actuator 4692 (e.g., a rotatable knob) that a user can manipulate to drive the inner catheter assembly (not visible in FIG. 46A) and the tip member 4620 in a distal direction. As shown in FIG. 46E, the forward drive mechanism 4690 can include a forward drive lead screw 4694, a lead screw coupling element 4693, a forward drive carriage 4695, and a forward drive driver 4696. The forward drive actuator 4692 can be operably (e.g., rotatably) coupled to the forward drive lead screw 4694 via the lead screw coupling element 4693, which in various embodiments can either be a head of the forward drive lead screw 4694 or a separate component. The forward drive lead screw 4694 is translationally locked to the forward drive carriage 4695, which itself is translationally locked relative to the housing 4632. The forward drive driver 4696 is translationally coupled to the forward drive carriage 4695, e.g., via a tongue-and-groove, slot-and-rail, or other suitable mechanism. The forward drive driver 4696 also includes a threaded female connection 4697 for engaging the forward drive lead screw 4694.

The forward drive driver 4696 is fixed to the inner catheter assembly 4610, which includes both the pusher catheter 4612 and the inner catheter 4616. The forward drive driver 4696 can be fixedly coupled to the inner catheter assembly 4610 either directly or via one or more intervening components. For example, in some embodiments the forward drive driver 4696 can be fixedly coupled to a valve body 4698 of the second flush port 4684. The pusher catheter 4612 can terminate within the valve body 4698 while the inner catheter 4616 can extend through the valve body 4698 and into an inner catheter driver 4699. The valve body 4698 and the inner catheter driver 4699 can be operably coupled via one or more overlapping tabs, flanges, pins, or the like such that the valve body 4698 and the inner catheter driver 4699 translate together. As a result, translation of the forward drive driver 4696 can induce translation of both the valve body 4698 and the inner catheter driver 4699, which in turn can cause both the pusher catheter 4612 and the inner catheter 4616 to translate.

In operation, rotation or other actuation of the forward drive actuator 4692 causes the forward drive lead screw 4694 to rotate (via the lead screw coupling element 4693). Rotation of the forward drive lead screw 4694 threads the forward drive lead screw 4694 into the threaded female connection 4697 of the forward drive driver 4696. Because the lead screw 4694 is translationally fixed to the forward drive carriage 4695, rotation of the lead screw 4694 within the threaded female connection 4697 drives the forward drive driver 4696 in the distal direction D relative to the housing 4632. Specifically, the forward drive driver 4696 moves distally relative to the forward drive carriage 4695 along the tongue-and-groove or other connection mechanism. Because the forward drive driver 4696 is fixed to the inner catheter assembly 4610 (e.g., both the pusher catheter 4612 and the inner catheter 4616) as described previously, movement of the forward drive driver 4696 distally drives the pusher catheter 4612 and the inner catheter 4616 in the distal direction, which in turn drives the tip member 4620 (FIG. 46A) distally. To the extent the aortic repair device is coupled to the tip member 4620 (e.g., via a tip capture mechanism described herein), this distal movement of the tip member 4620 drives (e.g., pulls) the aortic repair device coupled to the tip member 4620 “forward” away from the handle 4630.

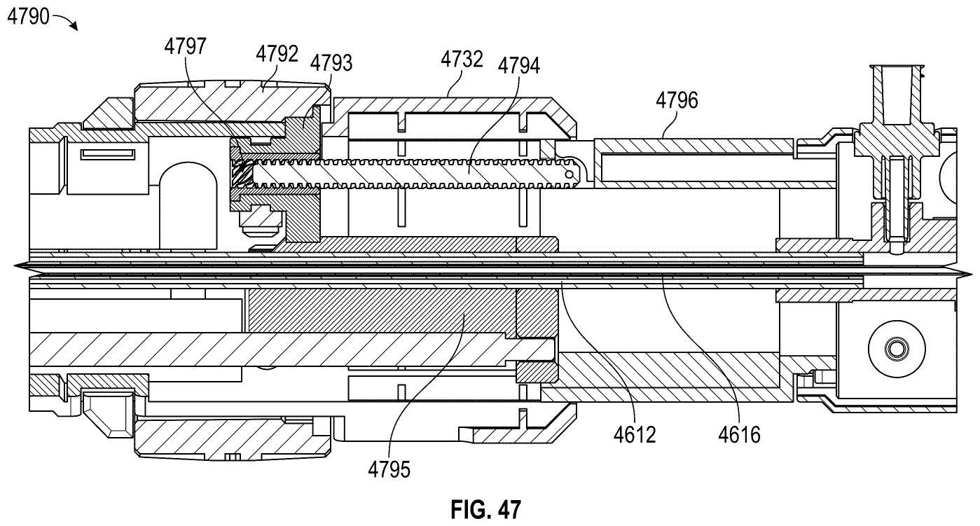

Forward drive mechanisms in accordance with embodiments of the present technology can have other configurations. For example, FIG. 47 is an enlarged cross-sectional view of a forward drive mechanism 4790 for use with a delivery system that is substantially similar to the delivery system 4600 of FIGS. 46A-46E. As shown in FIG. 47, the forward drive mechanism 4790 includes a forward drive actuator 4792, a forward drive lead screw 4794, a lead screw coupling element 4793, a forward drive carriage 4795, and a forward drive driver 4796. The lead screw coupling element 4793 and the forward drive carriage 4795 are translationally fixed to a housing 4732. Relative to the embodiment described with reference to FIGS. 46A-46E, however, the forward drive lead screw 4794 is not translationally fixed to the housing 4732. Instead, the lead screw coupling element 4793 includes a threaded female connection 4797 for engaging the forward drive lead screw 4794, and the forward drive lead screw is fixedly coupled to the forward drive driver 4796. Accordingly, rotation of the forward drive actuator 4792 rotates the lead screw coupling element 4793 and threads the forward drive lead screw 4794 further into the threaded female connection 4797, driving (e.g., pulling) the lead screw 4794 and the forward drive driver 4796 in the distal direction. Similar to the forward drive mechanism 4690 of FIGS. 46A-46E, this drives the pusher catheter 4612 and the inner catheter 4616 in the “forward” or distal direction.

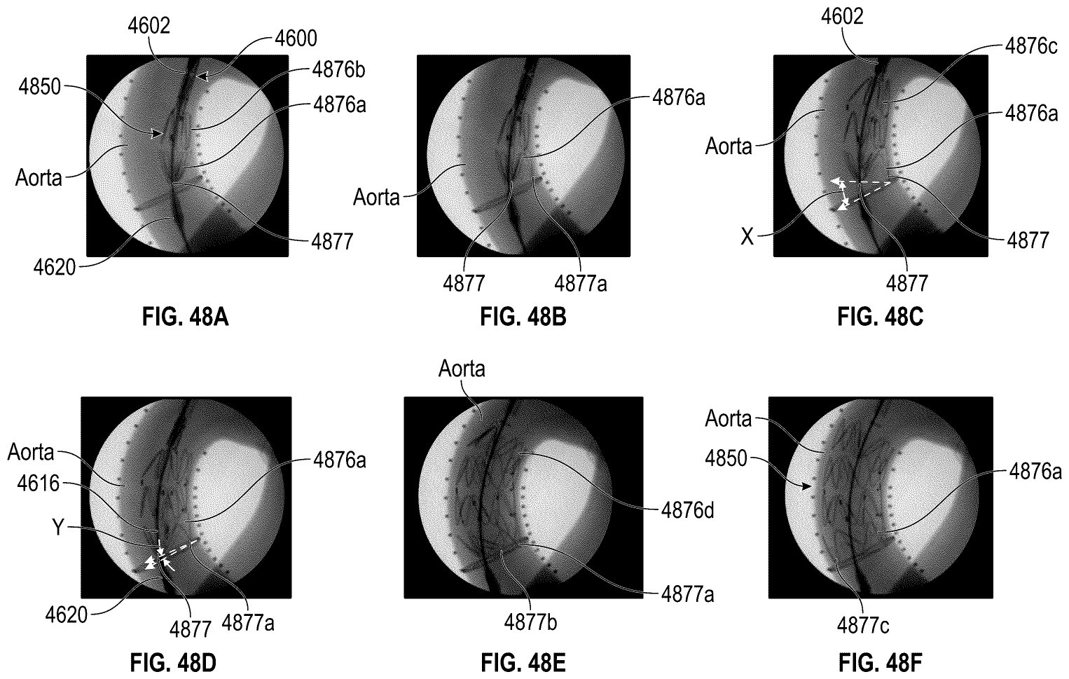

The forward drive mechanisms described herein can be used to enhance alignment and control deployment of vessel repair devices (e.g., stents, aortic repair devices disclosed herein) during a delivery procedure, especially when implanting devices in curved portions of vessels, such as the aortic arch. For example, the forward drive mechanisms can be used to adjust and/or enhance alignment of a leading edge of an aortic repair device within a target vessel by changing a tilt of a leading edge relative to the inner wall of the target vessel, thereby allowing a clinician to “square” the leading edge to the target vessel. That is, the leading edge of the aortic repair device can be positioned such that it extends in a line substantially straight across the target vessel from the lesser to the greater curvature (when taken in cross-section, e.g., aiming to have the leading end forming a right angle with the inner wall of the aorta at both the greater and lesser curvature), rather than the portion of the leading end of the aortic repair device near the greater curvature being positioned downstream from the portion of the leading end near the lesser curvature. FIGS. 48A-48F illustrate various stages of a procedure of deploying an aortic repair device 4850 within a model of an aorta of a human subject using the delivery system 4600 of FIGS. 46A-46E. Referring first to FIG. 48A, the delivery system 4600 can be advanced through the vasculature of the subject until the tip member 4620 and the aortic repair device 4850 are positioned proximate a target deployment location within the aorta. The outer catheter 4602 can then be retracted proximally to expose one or more stents of the aortic repair device 4850, as also shown in FIG. 48A. For example, in the illustrated embodiment the outer catheter 4602 has been retracted until a first stent 4876a and a second stent 4876b are exposed. This can be performed by rotating the actuator 4635, described above with reference to FIG. 46D. Of note, apices 4877 at the leading (e.g., proximal) end of the first stent 4876a are retained via a tip capture mechanism, and therefore the first stent 4876a does not fully radially expand even after the outer catheter 4602 is withdrawn.

Next, as shown FIG. 48B, one or more of the apices 4877 can be released from the tip capture mechanism by pulling a corresponding release knob 4642 illustrated in and described with reference to FIG. 46B. In the illustrated embodiment, a first apex 4877a (or first pair of apices 4877a) positioned toward an inner curve of the aorta is released while the other apices positioned toward an outer curve remain captured. In other embodiments, additional apices 4877 can be released, for example, by pulling additional release knobs 4642. The outer catheter 4602 can then be further retracted by further rotating the actuator 4635 (FIG. 46D) to expose a third stent 4876c, as shown in FIG. 48C.

As best shown in FIG. 48C, a leading (e.g., proximal) edge of the first stent 4876a (as defined by the apices 4877) is not square within the aorta at this stage of deployment. That is, the leading edge of the first stent 4876a does not occupy a plane that is perpendicular to a central longitudinal axis of the aorta, based on the curvature of the aorta. Instead, the leading edge of the first stent 4876a forms a first angle X relative to the plane that is perpendicular to the central longitudinal axis of the aorta. Accordingly, if the first stent 4876a were fully released in the position shown in FIG. 48C, the first stent 4876a would not be deployed squarely within the aorta.

To address this, the forward drive mechanism 4690 described with reference to FIG. 46E can be actuated (e.g., by rotating the forward drive actuator 4692). As explained previously, actuating the forward drive mechanism 4690 drives the pusher catheter 4612, the inner catheter 4616, and the tip member 4620 distally (e.g., “forward” toward the heart). Because some of the apices 4877 of the aortic repair device 4850 remain connected to the tip member 4620 via the tip capture mechanism, driving the tip member 4620 distally also pulls the apices 4877 that remain connected to the tip member 4620 “forward” toward the heart, as shown in FIG. 48D. As also shown in FIG. 48D, this squares or substantially squares the position of the first stent 4876a within the aorta. In particular, after operating the forward drive mechanism 4690, the leading edge of the first stent 4876a forms a second angle Y relative the plane that is perpendicular to the central axis of the aorta. The second angle Y is less than the first angle X shown in FIG. 48C, reflecting the improved squareness of the implant within the aorta. Indeed, in some embodiments the second angle Y is expected to be within about 10 degrees of zero, or within about 5 degrees of zero, or within 2 degrees of zero.

With the first stent 4876a square or at least substantially square in the aorta, additional apices 4877 can be released. For example, as shown in FIG. 48E, a second apex 4877b (or second set of apices 4877b) has been released, for example, by pulling a second one of the release knobs 4642 (FIG. 46B). As also shown in FIG. 48E, the outer catheter 4602 can be further retracted to expose a fourth stent 4876d. The third apex 4877c (or third set of apices 4877c) can then be released, as shown in FIG. 48F, such that the first stent 4876a is fully deployed within the aorta. Of note, by virtue of actuating the forward drive mechanism 4690, the leading edge of the first stent 4876a (and thus the aortic repair device 4850 itself) is squarely positioned within the aorta after deployment.

The forward drive mechanism 4690 is therefore expected to improve the accuracy of deploying the aortic repair device 4850 in the aorta and increase the potential sealing area between the aortic repair device 4850 and the aorta (by virtue of the enhanced alignment and increased available landing zone), which in turn is expected to enhance sealing and fixation of aortic repair devices to the opposing vessel wall. Further, by improving the deployment accuracy of the aortic repair device 4850, the forward drive mechanism 4690 reduces the likelihood that the aortic repair device 4850 would need to be repositioned within the vessel after deployment or recaptured, which decreases the risk of unnecessarily moving the device along the inner vessel wall and thus potentially damaging the already injured or diseased vessel during deployment of the aortic repair device.

Although FIGS. 48A-48F describe deploying the stents 4876 and the apices 4877 in a particular sequence, one skilled in the art will appreciate that the stents 4876 and apices 4877 can be deployed in other sequences. For example, in some embodiments the outer catheter 4602 can be retracted to expose each of the stents 4876a-4876d before releasing any of the apices 4877 of the first stent 4876a and operating the forward drive mechanism 4690. Accordingly, the present technology is not limited to the particular sequence described above with reference to FIGS. 48A-48E. Similarly, the forward drive mechanism 4690 can be used to deploy aortic repair devices with a different number of stents, including any of the stents described throughout this Detailed Description. Moreover, the delivery system 4600 with the forward drive mechanism 4690 can be used to deliver stents and repair devices to other tortuous vessels in addition to the aorta.

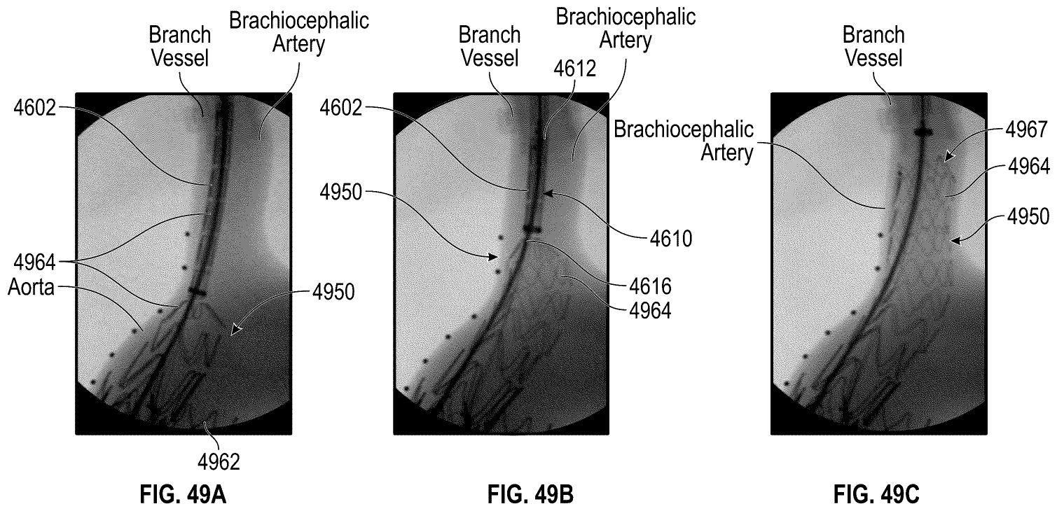

The forward drive mechanisms described herein can facilitate additional advantages during a delivery procedure. For example, in certain embodiments an aortic repair device may have a trailing (e.g., proximal) end portion that is deployed proximate a branch vessel. The forward drive mechanism can be used to compress the aortic repair device to reduce the likelihood that the trailing end portion overlaps or blocks blood flow to the branch vessel. For example, FIGS. 49A-49C illustrate stages of a procedure of deploying an aortic repair device 4950 in a model of an aorta and brachiocephalic artery of a human subject. FIG. 49A illustrates a first stage of deployment, after a body portion 4962 of the aortic repair device 4950 has been deployed in the aorta and while a segment of a leg portion 4964 of the aortic repair device 4950 remains within the outer catheter 4602. As shown in FIG. 49A, a branch vessel extends from the brachiocephalic artery.

FIG. 49B illustrates a second stage of deployment after the outer catheter 4602 has been further retracted to expose additional segments of the leg portion 4964. Of note, if the outer catheter 4602 were fully retracted and the leg portion 4964 fully deployed at the position shown in FIG. 49B, the leg portion 4964 may obstruct or at least partially obstruct the branch vessel. To address this, with the leading edge (not shown) of the aortic repair device fully deployed within the aorta, the forward drive mechanism 4690 can be actuated. Because the leading edge is fully released from any tip capture mechanism, the leading edge is not coupled to the tip member (not shown), the inner catheter 4616, or the pusher catheter 4612, and therefore is not driven forward in response to the forward drive mechanism being actuated. Rather, the forward drive mechanism pushes a trailing (e.g., distal) end of the aortic repair device forward, e.g., through a trailing end tip capture mechanism that couples the trailing end of the aortic repair device to a portion of the inner catheter assembly 4610 (e.g., a stopping member generally similar to the stopping member 321 described with reference to FIG. 3B). This compresses the overall axial footprint of the aortic repair device 4950 without changing a position of the leading edge of the aortic repair device 4950. As a result, and as shown in FIG. 49C, the trailing end portion 4967 of the leg 4964 of the aortic repair device 4950 is positioned within the brachiocephalic artery but does not obstruct the branch vessel following deployment. Of note, using the forward drive mechanism to drive the trailing edge of the aortic repair device forward can be done before or after exposing the trailing end of the aortic repair device by retracting the outer catheter 4602.

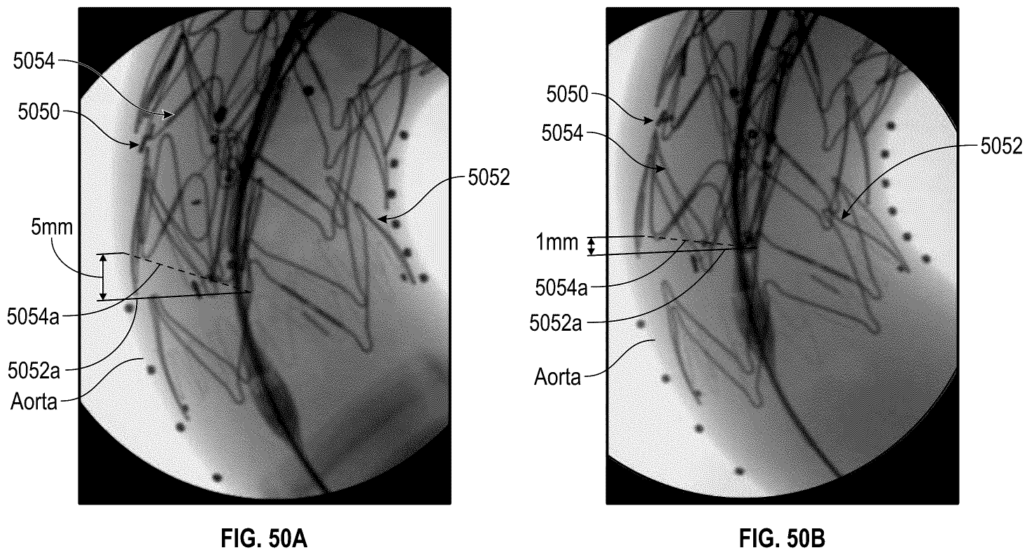

The forward drive mechanisms described herein can also be used to improve engagement or alignment between two or more components of an aortic repair system. Some aortic repair systems include modular components including a trunk and a limb, with the trunk and the limb being delivered and deployed separately. Examples of modular aortic repair systems are described in U.S. Provisional Patent Application No. 63/530,420, filed Aug. 2, 2023, the disclosure of which is incorporated by reference herein in its entirety. FIGS. 50A and 50B illustrate an example of using a forward drive mechanism to improve the alignment between a trunk and a limb of a modular aortic repair system. For example, FIG. 50A illustrates a modular aortic repair system 5050 having a trunk 5052 and a limb 5054 deployed within a model of an aorta of a human subject without using a forward drive mechanism during deployment of the limb. As shown, a leading (e.g., proximal) edge 5052a of one of the stents forming the trunk 5052 is marked with a solid line, and a leading (e.g., proximal) edge 5054b of one of the stents forming the limb 5054 is marked with a dashed line. As shown, the leading edge 5054a of the limb 5054 is not aligned with the leading edge 5052a of the trunk 5054. In contrast, FIG. 50B illustrates the modular aortic repair system 5050 deployed in the aorta after use of a forward drive mechanism to drive the portion of the leading edge 5054a of the limb 5054 that faces the greater aortic curve forward during deployment. As a result of using the forward drive mechanism, the leading edge 5054a of the limb 5054 is better aligned with the leading edge 5052a of the trunk 5052. Without intending to be bound by theory, better alignment between the leading edges 5052a and 5054a is expected to improve the engagement between the trunk 5052 and the limb 5054, which in turn may improve the effectiveness of the therapy and/or decrease the failure rate of the system.

### VIII. ADDITIONAL EXAMPLES

The following examples are illustrative of several embodiments of the present technology:

1. A delivery system for implanting an aortic repair device, comprising:
an outer catheter defining a lumen;
an inner catheter assembly extending at least partially through the lumen of the outer catheter;
a tip capture mechanism coupled to the inner catheter assembly and configured to releasably secure an end portion of the aortic repair device;
a handle including an actuator operably coupled to the outer catheter, wherein the actuator is actuatable to retract the outer catheter relative to the inner catheter assembly from a delivery position to a deployed position, wherein the outer catheter is positioned over and constrains the aortic repair device within the lumen in the delivery position, and wherein the outer catheter is withdrawn from over the aortic repair device in the deployed position such that the aortic repair device can expand; and
a release wire operably coupled to the tip capture mechanism, wherein the release wire is actuatable to release the end portion of the aortic repair device from the tip capture mechanism.

2. The delivery system of example 1 wherein the end portion of the aortic repair device is a leading end portion of the aortic repair device.

3. The delivery system of example 1 wherein the end portion of the aortic repair device is a trailing end portion of the aortic repair device.

4. The delivery system example 1 wherein the end portion of the aortic repair device is a leading end portion of the aortic repair device, wherein the aortic repair device includes a stent at the leading end portion, and wherein the tip capture mechanism is configured to releasably secure the aortic repair device.

5. The delivery system of example 4 wherein the tip capture mechanism comprises a loop threaded at least partially around the stent, and wherein the release wire is configured to extend through the loop to releasably secure the stent to the tip capture mechanism.

6. The delivery system of example 4 wherein the tip capture mechanism comprises a body defining a recess and a lumen extending through the body, wherein the recess is configured to receive a portion of the stent therein, and wherein the release wire is configured to extend through the lumen over the portion of the stent to releasably secure the stent to the tip capture mechanism.

7. The delivery system of any one of examples 1-6 wherein the release wire is one of a plurality of release wires, and wherein the release wires are individually actuatable to release a corresponding region of the end portion of the aortic repair device from the tip capture mechanism.

8. A delivery system for implanting a medical device having at least one stent with an end portion formed by a plurality of apices, wherein the delivery system comprises:
a handle;
a catheter assembly extending from the handle and sized and shaped to carry the medical device through a vasculature of a human patient toward a target deployment location within the vasculature, wherein the catheter assembly includes an outer catheter transitionable between delivery configuration in which it covers the medical device and a deployment configuration in which it at least partially uncovers the medical device;
a tip capture mechanism configured to releasably constrain at least some of the apices of the medical device when the outer catheter is in the deployment configuration, wherein the tip capture mechanism includes a loop threaded through one or more of the apices to radially constrain the one or more apices to another component of the catheter assembly; and
a release mechanism actuatable to release the constrained one or more apices from the tip capture mechanism.

9. The delivery system of example 8 wherein the loop is threaded through the one or more apices in an over-under pattern.

10. The delivery system of example 8 or example 9 wherein the loop is threaded through a single apex.

11. The delivery system of example 8 or example 9 wherein the loop is threaded through two or more of the apices.

12. The delivery system of any of examples 8-11 wherein the catheter assembly includes an inner catheter extending within the outer catheter and terminating at a tip member, and wherein the medical device is configured to be positioned around the inner catheter.

13. The delivery system of example 12 wherein the tip capture mechanism releasably constrains the at least some apices to the inner catheter and/or the tip member.

14. The delivery system of example 12 or example 13 wherein the tip member includes a retention pin, and wherein the loop is releasably secured to the retention pin.

15. The delivery system of example 14 wherein the release mechanism, when actuated, separates a tip member first portion from a tip member second portion to release the loop from the retention pin.

16. The delivery system of any of examples 8-15, further comprising a tip capture adjustment mechanism for adjusting a degree of constrained provided by the loop.

17. The delivery system of example 16 wherein the tip capture adjustment mechanism includes a rotatable shaft coupled to the loop, and wherein the shaft is configured such that (a) rotating the shaft in a first direction unwinds the loop from the shaft and reduces the amount of constraint provided by the loop, and (b) rotating the shaft in a second direction winds the loop around the shaft and increases the amount of constrain provided by the loop.

18. The delivery system of any of examples 8-17 wherein the medical device is an aortic repair device.

19. A delivery system for implanting a medical device having at least one stent with an end portion formed by a plurality of apices, wherein the delivery system comprises:
a handle;
a catheter extending from the handle and sized and shaped to carry the medical device through a vasculature of a human patient toward a target deployment location within the vasculature; and
a tip capture mechanism configured to releasably constrain at least some of the plurality of apices of the medical device, wherein the tip capture mechanism includes—
   a first capture element configured to releasably constrain a first subset of one or more of the plurality of apices that form the end portion of the stent,
   a second capture element configured to releasably constrain a second subset of one or more of the plurality of apices that form the end portion of the stent, the second subset being different than the first subset,
   a first release mechanism configured to selectively release the first subset of the one or more apices from being constrained by the first capture element, and
   a second release mechanism configured to selectively release the second subset of the one or more apices from being constrained by the second capture element,
   wherein the first release mechanism and the second release mechanism are independently actuatable.

20. The delivery system of example 19 wherein the first capture element comprises a first loop composed of a first thread, fiber, or cable, and wherein the second capture element comprises a second loop composed of a second thread, fiber, or cable.

21. The delivery system of example 19 or example 20 wherein the first subset of apices constrained by the first capture element includes at least a first apex and a second apex, and wherein the second subset of apices constrained by the second capture element includes at least a third apex and a fourth apex.

22. The delivery system of example 21 wherein the first apex and the second apex are located adjacent to each other around a periphery of the end portion of the stent.

23. The delivery system of example 21 wherein the first apex and the second apex are spaced apart around a periphery of the end portion of the stent by at least one other apex of the plurality of apices.

24. The delivery system of any of examples 19-23 wherein the first release mechanism includes a first release wire extending between the handle and the first capture element, and wherein the second release mechanism includes a second release wire extending between the handle and the second capture element.

25 The delivery system of any of examples 19-24 wherein the catheter is an outer catheter having a lumen, and wherein the delivery system further comprises:
an inner catheter extending through the lumen, wherein the medical device is positionable within the lumen between the outer catheter and the inner catheter; and
an actuation mechanism actuatable to retract the outer catheter relative to the inner catheter from a delivery configuration in which the outer catheter covers the medical device to a deployment configuration in which the medical device uncovers the medical device.

26. The delivery system of example 25 wherein the tip capture mechanism is configured to constrain the medical device from fully radially expanding when the outer catheter is retracted to the deployment configuration.

27. The delivery system of example 25 or example 26 wherein the inner catheter terminates at a tip member, and wherein the first capture element and second capture element releasably constrain the corresponding first subset and second subset of apices to the tip member.

28. The delivery system of any of examples 25-27, further comprising a forward drive mechanism configured to translate the inner catheter in a distal direction and relative to the outer catheter.

29. The delivery system of any of examples 19-28 wherein the medical device is an aortic repair device.

30. A delivery system for implanting a medical device having at least one stent with an end portion formed by a plurality of apices, wherein the delivery system comprises:
a handle;
an outer catheter defining a lumen;
an inner catheter extending through the lumen of the outer catheter and terminating at a tip member, wherein the medical device is configured to be positioned within the lumen between the outer catheter and the inner catheter;
an actuation mechanism actuatable to retract the outer catheter relative to the inner catheter from a delivery configuration in which the outer catheter covers the medical device to a deployment configuration in which the medical device uncovers the medical device;
a tip capture mechanism configured to releasably constrain at least some of the plurality of apices of the medical device to the inner catheter when the outer catheter is in both the delivery configuration and the deployment configuration, wherein the tip capture mechanism includes—
   a first loop configured to releasably constrain a first subset of one or more of the plurality of apices that form the end portion of the stent to the inner catheter,
   a second loop configured to releasably constrain a second subset of one or more of the plurality of apices that form the end portion of the stent to the inner catheter, the second subset being different than the first subset,
   a first release mechanism configured to selectively release the first subset of the one or more apices from being constrained by the first loop, and
   a second release mechanism configured to selectively release the second subset of the one or more apices from being constrained by the second capture element; and
a forward drive mechanism configured to drive the inner catheter in a distal direction relative to the outer catheter.

31. The delivery system of example 30 wherein the handle includes a first handle portion and a second handle portion at least partially nested within the first handle portion, and wherein the forward drive mechanism, when actuated, translates the second handle portion relative to the first handle portion.

32. The delivery system of example 30 or example 31 wherein the forward drive mechanism comprises:
a forward drive driver operably coupled to the inner catheter; and
a forward drive actuator actuatable to translate the forward drive driver distally within the handle to causes a corresponding distal movement of the inner catheter.

33. The delivery system of example 32 wherein the forward drive mechanism further includes a lead screw operably coupled to the forward drive driver such that rotation of the lead screw causes a corresponding translational movement of the forward drive driver, and wherein the forward drive mechanism is configured such that actuating the forward drive actuator rotates the lead screw.

34 The delivery system of example 32 or example 33 wherein the forward drive actuator comprises a rotatable knob operably coupled to the handle.

35. The delivery system of any of examples 30-34, further comprising a pusher catheter extending at least partially between the inner catheter and the outer catheter, wherein the forward drive mechanism is further configured to drive the pusher catheter in a distal direction relative to the outer catheter.

36. The delivery system of any of examples 30-35 wherein the medical device is an aortic repair device.

37. A method of deploying an aortic repair device having at least one stent with an end portion defined by a plurality of apices within an aorta of a human subject using an aortic repair device delivery system, the method comprising:
intravascularly advancing a catheter assembly of the delivery system toward a target site in the aorta, wherein the catheter assembly carries the aortic repair device, and wherein at least some of the apices are radially constrained by a tip capture mechanism of the delivery system;
with the aortic repair device positioned proximate the target site, releasing a first constrained subset of one or more of the plurality of apices while retaining a second subset of one or more of the plurality of apices, wherein the released first subset of apices extend radially outwardly;
after releasing the first subset of apices and while retaining the second subset of apices, actuating a forward drive mechanism of the delivery system to change a tilt of the aortic repair device within the aorta; and
after changing the tilt of the aortic repair device, releasing the constrained subset of apices.

38 The method of example 37 wherein changing the tilt of the aortic repair device includes moving the retained second subset of apices distally without substantially moving the released first subset of apices.

39 The method of example 38 or example 39 wherein the target site is at the ascending aorta, and wherein the first subset of apices includes one or more apices facing an inner curve of the ascending aorta and the second subset of apices includes one or more apices facing an outer curve of the ascending aorta.

40 The method of any of examples 37-39 wherein the catheter assembly includes an outer catheter and an inner catheter extending within the outer catheter, and wherein the aortic repair device is positioned between the outer catheter and the inner catheter while intravascularly advancing the aortic repair device toward the target site.

41. The method of example 40, further comprising, after positioning the aortic repair device proximate the target site and before releasing the first constrained subset of apices, retracting the outer catheter relative to the inner catheter to uncover the aortic repair device.

42. The method of any of examples 37-41 wherein the first subset of apices is releasably constrained by a first loop of a tip capture mechanism, and wherein the second subset of apices is releasably constrained by a second loop of the tip capture mechanism.

43. The method of any of examples 37-42 wherein the first subset of apices includes a single first apex, and wherein the second subset of apices includes a single second apex.

44. The method of any of examples 37-42 wherein the first subset of apices includes at least a first apex and a second apex, and wherein the second subset of apices includes at least a third apex and a fourth apex.

45. A delivery system for implanting a vessel repair device within a target vessel of a patient, the delivery system comprising:
an outer catheter defining a lumen;
an inner catheter extending at least partially through the lumen of the outer catheter, wherein at least a portion of the vessel repair device is releasably coupled to the inner catheter;
a handle including an actuator operably coupled to the outer catheter, wherein:
   the actuator is actuatable to retract the outer catheter relative to the inner catheter from a delivery position to a deployed position,
   in the delivery position, the outer catheter is positioned over and constrains the vessel repair device within the lumen, and
   in the deployed position the outer catheter is withdrawn from over the vessel repair device; and
wherein the delivery system is configured to alter an orientation and/or tilt of the vessel repair device within the target vessel.

46. The delivery system of example 45 wherein the delivery system is configured such that the orientation and/or tilt of the vessel repair device is actively adjustable when the outer catheter is in the delivery position.

47. The delivery system of example 46 wherein the inner catheter is configured to adjust orientation while the vessel repair device remains releasably coupled to the inner catheter such that movement of the inner catheter alters the orientation and/or tilt of the vessel repair device.

48. The delivery system of example 47, further comprising a tendon operably coupled to the inner catheter, wherein the tendon is manipulatable to flex at least a portion of the inner catheter in a first direction to alter the orientation and/or tilt of the vessel repair device.

49. The delivery system of example 48 wherein the tendon is a first tendon, and wherein the delivery system further comprises a second tendon operably coupled to the inner catheter and manipulatable to flex at least a portion of the inner catheter in a second direction, different than the first direction.

50. The delivery system of any of examples 47-49 wherein the inner catheter comprises a hypotube having a relief pattern designed to control a degree and/or a direction of adjustability for the inner catheter.

51. The delivery system of example 46, further comprising a detachable tether operably coupled to the vessel repair device, wherein the detachable tether is manipulatable to alter the orientation and/or tilt of the vessel repair device.

52. The delivery system of example 45 wherein the delivery system is configured such that the orientation and/or tilt of the vessel repair device is automatically adjustable.

53. The delivery system of example 52, further comprising a shaping feature coupled to the inner catheter, wherein the shaping feature is configured to automatically control an orientation of the inner catheter.

54. The delivery system of example 53 wherein the shaping feature comprises a nitinol sleeve.

55. The delivery system of example 53 or example 54 wherein the shaping feature includes a first bend region at which the shaping feature is configured to flex in a first direction to bend the inner catheter in the first direction, and a second bend region at which the shaping feature is configured to flex in a second direction, different than the first direction, to bend the inner catheter in the second direction.

56 The delivery system of any of examples 53-55 wherein the shaping feature is further configured to automatically control an orientation of the outer catheter in addition to controlling an orientation of the inner catheter.

57 The delivery system of example 52 wherein the outer catheter is shape set such that the outer catheter is configured to bend in predetermined directions and/or to a predetermined degree.

58. The delivery system of example 52, further comprising a detachable wire coupled to the vessel repair device, wherein the detachable wire at least partially constrains a radial expansion of a portion of the vessel repair device to alter the orientation and/or tilt of the vessel repair device relative to a target vessel.

59. The delivery system of example 58 wherein the detachable wire is coupled to an internal portion of the vessel repair device.

60. The delivery system of example 58 wherein the detachable wire is coupled to an external portion of the vessel repair device.

61 The delivery system of example 52, further comprising a forward drive mechanism for altering the orientation and/or tilt of the vessel repair device within the target vessel.

62. A method of deploying an implantable vessel repair device within a target vessel of a human subject using a vessel repair device delivery system, the method comprising:
intravascularly advancing a catheter assembly of the delivery system toward a target site in the target vessel of the patient, wherein the catheter assembly carries the vessel repair device;
with the vessel repair device positioned proximate the target site and at last partially retained by the catheter assembly, adjusting an orientation and/or tilt of an end portion of the vessel repair device within the target vessel to square the end portion relative to the target vessel; and
releasing the vessel repair device from the catheter assembly at the adjusted orientation and/or tilt.

63. The method of example 62 wherein adjusting the orientation and/or tilt of the vessel repair device includes actively adjusting the orientation and/or tilt using one or more active adjustment mechanisms of the delivery system.

64. The method of example 62 wherein adjusting the orientation and/or tilt of the vessel repair device includes automatically adjusting the orientation and/or tilt of the vessel repair device.

65. The method of any of examples 62-64, further comprising at least partially deploying the vessel repair device at the target site before adjusting the orientation and/or tilt of the vessel repair device.

66. The method of any of examples 62-65 wherein adjusting the orientation and/or tilt of the vessel repair device comprises moving the vessel repair device in a manner to avoid the vessel repair device from extending across an opening of one or more branch vessels proximate the target site.

67. The method of any of examples 62-65 wherein adjusting the orientation and/or tilt of the vessel repair device comprises aligning a leading end portion the vessel repair device with a plane of a sinotubular junction of the aorta at the target site.

68. The method of any of examples 62-65 wherein adjusting the orientation and/or tilt of the vessel repair device comprises positioning an end portion of the vessel repair device such that the end portion is generally orthogonal to an inner wall of an aortic arch proximate a greater curvature of the aortic arch.

69. The method of any of examples 62-65 wherein adjusting the orientation and/or tilt of the vessel repair device comprises positioning an end portion of the vessel repair device such that the end portion is generally orthogonal to an inner wall of a stent implanted in the target vessel.

### IX. CONCLUSION

The above detailed description of embodiments of the technology are not intended to be exhaustive or to limit the technology to the precise form disclosed above. Although specific embodiments of, and examples for, the technology are described above for illustrative purposes, various equivalent modifications are possible within the scope of the technology as those skilled in the relevant art will recognize. For example, although steps are presented in a given order, alternative embodiments can perform steps in a different order. The various embodiments described herein can also be combined to provide further embodiments.

From the foregoing, it will be appreciated that specific embodiments of the technology have been described herein for purposes of illustration, but well-known structures and functions have not been shown or described in detail to avoid unnecessarily obscuring the description of the embodiments of the technology. Where the context permits, singular or plural terms can also include the plural or singular term, respectively.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications can be made without deviating from the technology. Further, while advantages associated with some embodiments of the technology have been described in the context of those embodiments, other embodiments can also exhibit such advantages, and not all embodiments need necessarily exhibit such advantages to fall within the scope of the technology. Accordingly, the disclosure and associated technology can encompass other embodiments not expressly shown or described herein.

1. A delivery system for implanting an aortic repair device, comprising:
an outer catheter defining a lumen;
an inner catheter assembly extending at least partially through the lumen of the outer catheter;
a tip capture mechanism coupled to the inner catheter assembly and configured to releasably secure an end portion of the aortic repair device;
a handle including an actuator operably coupled to the outer catheter, wherein the actuator is actuatable to retract the outer catheter relative to the inner catheter assembly from a delivery position to a deployed position, wherein the outer catheter is positioned over and constrains the aortic repair device within the lumen in the delivery position, and wherein the outer catheter is withdrawn from over the aortic repair device in the deployed position such that the aortic repair device can expand; and
a release wire operably coupled to the tip capture mechanism, wherein the release wire is actuatable to release the end portion of the aortic repair device from the tip capture mechanism.

2. The delivery system of claim 1 wherein the end portion of the aortic repair device is a leading end portion of the aortic repair device.

3. The delivery system of claim 1 wherein the end portion of the aortic repair device is a trailing end portion of the aortic repair device.

4. The delivery system claim 1 wherein the end portion of the aortic repair device is a leading end portion of the aortic repair device, wherein the aortic repair device includes a stent at the leading end portion, and wherein the tip capture mechanism is configured to releasably secure the aortic repair device.

5. The delivery system of claim 4 wherein the tip capture mechanism comprises a loop threaded at least partially around the stent, and wherein the release wire is configured to extend through the loop to releasably secure the stent to the tip capture mechanism.

6. The delivery system of claim 4 wherein the tip capture mechanism comprises a body defining a recess and a lumen extending through the body, wherein the recess is configured to receive a portion of the stent therein, and wherein the release wire is configured to extend through the lumen over the portion of the stent to releasably secure the stent to the tip capture mechanism.

7. The delivery system of claim 1 wherein the release wire is one of a plurality of release wires, and wherein the release wires are individually actuatable to release a corresponding region of the end portion of the aortic repair device from the tip capture mechanism.

8. A delivery system for implanting a medical device having at least one stent with an end portion formed by a plurality of apices, wherein the delivery system comprises:
a handle;
a catheter assembly extending from the handle and sized and shaped to carry the medical device through a vasculature of a human patient toward a target deployment location within the vasculature, wherein the catheter assembly includes an outer catheter transitionable between delivery configuration in which it covers the medical device and a deployment configuration in which it at least partially uncovers the medical device;
a tip capture mechanism configured to releasably constrain at least some of the apices of the medical device when the outer catheter is in the deployment configuration, wherein the tip capture mechanism includes a loop threaded through one or more of the apices to radially constrain the one or more apices to another component of the catheter assembly; and
a release mechanism actuatable to release the constrained one or more apices from the tip capture mechanism.

9. The delivery system of claim 8 wherein the loop is threaded through the one or more apices in an over-under pattern.

10. The delivery system of claim 8 wherein the loop is threaded through a single apex.

11. The delivery system of claim 8 wherein the loop is threaded through two or more of the apices.

12. The delivery system of claim 8 wherein the catheter assembly includes an inner catheter extending within the outer catheter and terminating at a tip member, and wherein the medical device is configured to be positioned around the inner catheter.

13. The delivery system of claim 12 wherein the tip capture mechanism releasably constrains the at least some apices to the inner catheter and/or the tip member.

14. The delivery system of claim 12 wherein the tip member includes a retention pin, and wherein the loop is releasably secured to the retention pin.

15. The delivery system of claim 14 wherein the release mechanism, when actuated, separates a tip member first portion from a tip member second portion to release the loop from the retention pin.

16. The delivery system of claim 8, further comprising a tip capture adjustment mechanism for adjusting a degree of constrained provided by the loop.

17. The delivery system of claim 16 wherein the tip capture adjustment mechanism includes a rotatable shaft coupled to the loop, and wherein the shaft is configured such that (a) rotating the shaft in a first direction unwinds the loop from the shaft and reduces the amount of constraint provided by the loop, and (b) rotating the shaft in a second direction winds the loop around the shaft and increases the amount of constrain provided by the loop.

18. The delivery system of claim 8 wherein the medical device is an aortic repair device.

19. A delivery system for implanting a medical device having at least one stent with an end portion formed by a plurality of apices, wherein the delivery system comprises:
a handle;
a catheter extending from the handle and sized and shaped to carry the medical device through a vasculature of a human patient toward a target deployment location within the vasculature; and
a tip capture mechanism configured to releasably constrain at least some of the plurality of apices of the medical device, wherein the tip capture mechanism includes—
a first capture element configured to releasably constrain a first subset of one or more of the plurality of apices that form the end portion of the stent,
a second capture element configured to releasably constrain a second subset of one or more of the plurality of apices that form the end portion of the stent, the second subset being different than the first subset,
a first release mechanism configured to selectively release the first subset of the one or more apices from being constrained by the first capture element, and
a second release mechanism configured to selectively release the second subset of the one or more apices from being constrained by the second capture element,
wherein the first release mechanism and the second release mechanism are independently actuatable.

20. The delivery system of claim 19 wherein the first capture element comprises a first loop composed of a first thread, fiber, or cable, and wherein the second capture element comprises a second loop composed of a second thread, fiber, or cable.

21. The delivery system of claim 19 wherein the first subset of apices constrained by the first capture element includes at least a first apex and a second apex, and wherein the second subset of apices constrained by the second capture element includes at least a third apex and a fourth apex.

22. The delivery system of claim 21 wherein the first apex and the second apex are located adjacent to each other around a periphery of the end portion of the stent.

23. The delivery system of claim 21 wherein the first apex and the second apex are spaced apart around a periphery of the end portion of the stent by at least one other apex of the plurality of apices.

24. The delivery system of claim 19 wherein the first release mechanism includes a first release wire extending between the handle and the first capture element, and wherein the second release mechanism includes a second release wire extending between the handle and the second capture element.

25. The delivery system of claim 19 wherein the catheter is an outer catheter having a lumen, and wherein the delivery system further comprises:
an inner catheter extending through the lumen, wherein the medical device is positionable within the lumen between the outer catheter and the inner catheter; and
an actuation mechanism actuatable to retract the outer catheter relative to the inner catheter from a delivery configuration in which the outer catheter covers the medical device to a deployment configuration in which the medical device uncovers the medical device.

26. The delivery system of claim 25 wherein the tip capture mechanism is configured to constrain the medical device from fully radially expanding when the outer catheter is retracted to the deployment configuration.

27. The delivery system of claim 25 wherein the inner catheter terminates at a tip member, and wherein the first capture element and second capture element releasably constrain the corresponding first subset and second subset of apices to the tip member.

28. The delivery system of claim 25, further comprising a forward drive mechanism configured to translate the inner catheter in a distal direction and relative to the outer catheter.

29. The delivery system of claim 19 wherein the medical device is an aortic repair device.

30. A delivery system for implanting a medical device having at least one stent with an end portion formed by a plurality of apices, wherein the delivery system comprises:
a handle;
an outer catheter defining a lumen;
an inner catheter extending through the lumen of the outer catheter and terminating at a tip member, wherein the medical device is configured to be positioned within the lumen between the outer catheter and the inner catheter;
an actuation mechanism actuatable to retract the outer catheter relative to the inner catheter from a delivery configuration in which the outer catheter covers the medical device to a deployment configuration in which the medical device uncovers the medical device;
a tip capture mechanism configured to releasably constrain at least some of the plurality of apices of the medical device to the inner catheter when the outer catheter is in both the delivery configuration and the deployment configuration, wherein the tip capture mechanism includes—
a first loop configured to releasably constrain a first subset of one or more of the plurality of apices that form the end portion of the stent to the inner catheter,
a second loop configured to releasably constrain a second subset of one or more of the plurality of apices that form the end portion of the stent to the inner catheter, the second subset being different than the first subset,
a first release mechanism configured to selectively release the first subset of the one or more apices from being constrained by the first loop, and
a second release mechanism configured to selectively release the second subset of the one or more apices from being constrained by the second capture element; and
a forward drive mechanism configured to drive the inner catheter in a distal direction relative to the outer catheter.

31. The delivery system of claim 30 wherein the handle includes a first handle portion and a second handle portion at least partially nested within the first handle portion, and wherein the forward drive mechanism, when actuated, translates the second handle portion relative to the first handle portion.

32. The delivery system of claim 30 wherein the forward drive mechanism comprises:
a forward drive driver operably coupled to the inner catheter; and
a forward drive actuator actuatable to translate the forward drive driver distally within the handle to causes a corresponding distal movement of the inner catheter.

33. The delivery system of claim 32 wherein the forward drive mechanism further includes a lead screw operably coupled to the forward drive driver such that rotation of the lead screw causes a corresponding translational movement of the forward drive driver, and wherein the forward drive mechanism is configured such that actuating the forward drive actuator rotates the lead screw.

34. The delivery system of claim 32 wherein the forward drive actuator comprises a rotatable knob operably coupled to the handle.

35. The delivery system of claim 30, further comprising a pusher catheter extending at least partially between the inner catheter and the outer catheter, wherein the forward drive mechanism is further configured to drive the pusher catheter in a distal direction relative to the outer catheter.

36. The delivery system of claim 30 wherein the medical device is an aortic repair device.

37-69. (canceled)
